(19) United States
(12) Patent Application Publication
(10) Pub. No.: US 2026/0258052 A1
(43) Pub. Date: Sep. 3, 2026

(54) A UNIFIED STRATEGY FOR THE TOTAL SYNTHESES OF ERIBULIN AND A MACROLACTAM ANALOGUE OF HALICHONDRIN B

(71) Applicant: William Marsh Rice University, Houston, TX (US)

(72) Inventors: Kyriacos C. NICOLAOU, Houston, TX (US); Saiyong PAN, Houston, TX (US); Qiuji YE, Houston, TX (US); Yogesh Gangaram SHELKE, Houston, TX (US); Yifan ZHAO, Houston, TX (US)

(73) Assignee: William Marsh Rice University, Houston, TX (US)

(21) Appl. No.: 18/861,750

(22) PCT Filed: May 1, 2023
(86) PCT No.: PCT/US2023/066455

§ 371 Date: Oct. 30, 2024

Related U.S. Application Data
(60) Provisional application No. 63/337,093, filed on Apr. 30, 2022.

(51) Int. Cl. C07D 493/22; A61K 31/357; A61K 31/7048; A61K 45/06; C07H 9/02

(52) U.S. Cl. CPC C07D 493/22; A61K 31/357; A61K 31/7048; A61K 45/06; C07H 9/02

(57) ABSTRACT

As disclosed here, the present disclosure provides compounds of the formula: wherein the variables are as defined herein. These macrocycles can be used to as chemotherapeutic agents.

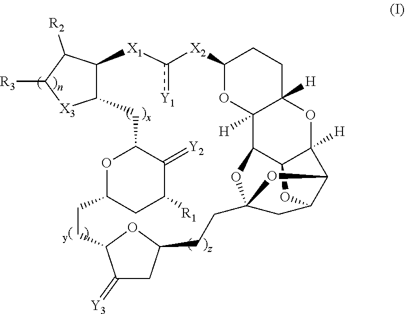

### CLAIM OF PRIORITY

This application claims the benefit of priority to U.S. Provisional Application No. 63/337,093, filed on Apr. 30, 2022, the entire contents of which are hereby incorporated by reference.

### BACKGROUND

### 1. Field

This disclosure relates to the fields of medicine, pharmacology, chemistry, and oncology. In particular, new compounds, compositions, methods of treatment, and methods of synthesis relating to analogs of eributin are disclosed.

### 2. Description of Related Art

The halichondrins, as a family of potent antitumor natural products,¹ have attracted considerable attention from synthetic organic chemists in academia² and the pharmaceutical industry.³ The pioneering work of Kishi and coworkers,^{2a} combined with the endeavors from Eisai,⁴ led to the discovery and development of eribulin (trade name of the clinically used mesylate salt Halaven), an analogue inspired by the structure and biological properties of halichondrin B, as a successful clinical drug against metastatic breast cancer and liposarcoma.⁵ Following the recent total syntheses of halichondrin B (1, Example 1)⁶ and norhalichondrin B (2, Example 1),⁷ through the ‘reverse approach’ to cyclic ether structural motifs (forming the C—O bond first followed by C—C bond construction) and employing convergent synthetic strategies, new methods need to be developed.

The so far reported syntheses of eribulin and other analogues of halichondrin B (1) by Eisai through two alternative synthetic sequences are highlighted in Example L.^{3d} Further medicinal chemistry (structure-activity relationship) studies⁴ with halichondrin B (1) analogues pointed to fragment A′ of eribulin and fragment ABCDEFG of halichondrin B as critical structural motifs of these molecules for structural modifications directed toward optimization of their biological and pharmacological properties. Thus, from the medicinal chemistry point of view, a late-stage installation of fragment A′ (e.g., 7a and 7b, Example 1) could improve the synthetic efficiency for the preparation of related designed analogues. With these considerations and the previously developed synthetic methods in mind, we envisioned a unified synthetic strategy for the total synthesis of eribulin (all-carbon macrocycle), and other related macrolactone and macrolactam analogues of the halichondrins. Such strategy may facilitate the synthesis of multiple designed analogues, thus providing a new opportunity for the discovery and optimization of next generation anticancer lead compounds in the area.

Therefore, further analogs and methods to obtain them are needed. This invention was funded in part by the Robert A. Welch Foundation under Welch Grant No. C-1819.

### SUMMARY

In some aspects, the present disclosure provides compounds that are halichondrins analogs. In some embodiments, the compounds are further defined as:

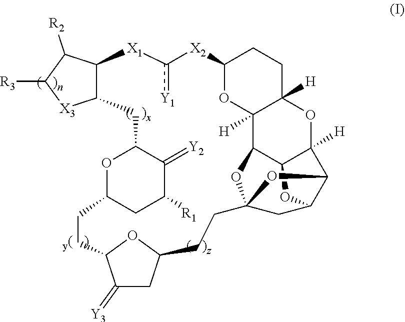

wherein:
   X₁, X₂, and X₃ are each independently a covalent bond, alkanediyl_{(C≤8)}, substituted alkanediyl_{(C≤8)}, NRₐ, O, and S; wherein:
      Rₐ is hydrogen, alkyl_{(C≤8)}, or substituted alkyl_{(C≤8)};
   Y₁, Y₂, and Y₃ are each independently CR_{b}R_{b}′, OR_{b}″, S, or NR_{c}, wherein
      R_{b}, R_{b}′, and R_{c} are each independently hydrogen, alkyl, or substituted alkyl; and
      R_{b}″ is absent, hydrogen, alkyl_{(C≤8)}, substituted alkyl_{(C≤8)}, acyl_{(C≤8)}, substituted acyl_{(C≤8)},
   x, y, and z are each independently 1, 2, or 3;
   R₁ is hydrogen, alkyl_{(C≤8)}, or substituted alkyl_{(C≤8)};
   R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamin_{(C≤12)}, acyl_{(C≤12)}, acyloxy_{(C≤12)}, amido_{(C≤12)}, or a substituted version thereof; or either two R₃ groups and R₂ and R are taken together to form one or more cycloalkanediyl_{(C≤18)}, arenediyl_{(C≤18)}, heteroarenediyl_{(C≤18)}, heterocycloalkanediyl_{(C≤18)}, or a substituted version thereof; or a group of the formula: —AR₃′, wherein A is alkanediyl_{(C≤8)} or substituted alkanediyl_{(C≤8)}; and R₃′ is NR_{d}C(X₄)NR_{d}′R″, wherein X₄ is O or NRₑ, wherein Rₑ is hydrogen, alkyl_{(C≤8)}, or substituted alkyl_{(C≤8)}, and R_{d}, R_{d}′, and R_{d}″ are each independently hydrogen, alkyl_{(C≤8)}, or substituted alkyl_{(C≤8)}; and
   n is 1 or 2; provided that for each n, then each R₃ is separately selected;
provided that the compound is not eributin;
or a pharmaceutically acceptable salt thereof.

In some embodiments, the compounds are further defined as:

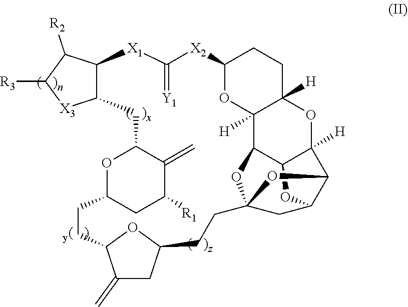

wherein:
   X₁, X₂, and X₃ are each independently a covalent bond, alkanediyl_{(C≤8)}, substituted alkanediyl_{(C≤8)}, NRₐ, O, and S; wherein:
      Rₐ is hydrogen, alkyl_{(C≤8)}, or substituted alkyl_{(C≤8)};
   Y₁ is CR_{b}R_{b}′, O, S, or NRₑ, wherein
      R_{b}, R_{b}′, and Rₑ are each independently hydrogen, alkyl_{(C≤8)}, or substituted alkyl_{(C≤8)};
   x, y, and z are each independently 1, 2, or 3;
   R₁ is hydrogen, alkyl_{(C≤8)}, or substituted alkyl_{(C≤8)};
   R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamino_{(C≤12)}, acyl_{(C≤12)}, acyloxy_{(C≤12)}, amido_{(C≤12)}, or a substituted version thereof; or either two R₃ groups and R₂ and R₃ are taken together to form one or more cycloalkanediyl_{(C≤18)}, arenediyl_{(C≤18)}, heteroarenediyl_{(C≤18)}, heterocycloalkanediyl_{(C≤18)}, or a substituted version thereof; or a group of the formula: —AR₃′, wherein A is alkanediyl_{(C≤8)} or substituted alkanediyl_{(C≤8)}; and R₃′ is NR_{d}C(X₄)NR_{d}′R_{d}″, wherein X₄ is O or NRₑ, wherein Rₑ is hydrogen, alkyl_{(C≤6)}, or substituted alkyl_{(C≤6)}, and R_{d}, R_{d}′, and R_{d}″ are each independently hydrogen, alkyl_{(C≤6)}, or substituted alkyl_{(C≤6)}; and
   n is 1 or 2; provided that for each n, then each R₃ is separately selected;
provided that the compound is not eributin;
or a pharmaceutically acceptable salt thereof.

In some embodiments, the compounds are further defined as:

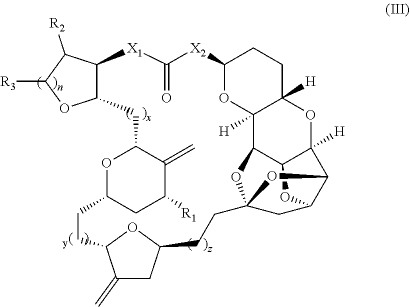

wherein:
   X₁ and X₂ is a covalent bond, alkanediyl_{(C≤8)}, substituted alkanediyl_{(C≤8)}, NRₐ, O, and S; wherein:
      Rₐ is hydrogen, alkyl_{(C≤8)}, or substituted alkyl_{(C≤8)};
   x, y, and z are each independently 1, 2, or 3;
   R₁ is hydrogen, alkyl_{(C≤8)}, or substituted alkyl_{(C≤8)};
   R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamnino_{(C≤12)}, acyl_{(C≤12)}, acyloxy_{(C≤12)}, amnido_{(C≤12)}, or a substituted version thereof; or either two R₃ groups and R₂″ and R₃ are taken together to form one or more cycloalkanediyl_{(C≤18)}, arenediyl_{(C≤18)}, heteroarenlediyl_{(C≤8)}, heterocycloalkanediyl_{(C≤12)}, or a substituted version thereof; or a group of the formula: —AR₃′, wherein A is alkanediyl_{(C≤8)} or substituted alkanediyl_{(C≤18)}; and R₃′ is NR_{d}C(X₄)NR_{d}′R_{d}″, wherein X₄ is O or NRₑ, wherein Rₑ is hydrogen, alkyl_{(C≤6)}, or substituted alkyl_{(C≤6)}, and R_{d}, R_{d}′, and R_{d}″ are each independently hydrogen, alkyl_{(C≤6)}, or substituted alkyl_{(C≤6)}; and
   n is 1 or 2; provided that for each n, then each R₃ is separately selected;
provided that the compound is not eributin;
or a pharmaceutically acceptable salt thereof.

In some embodiments, the compounds are further defined as:

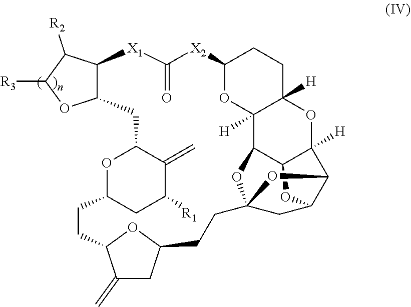

wherein:
   X₁ and X₂ is a covalent bond, alkanediyl_{(C≤8)}, substituted alkanediyl_{(C≤8)}, NRₐ, O, and
   S; wherein:
      Rₐ is hydrogen, alkyl_{(C≤8)}, or substituted alkyl_{(C≤8)};
   R₁ is hydrogen, alkyl_{(C≤8)}, or substituted alkyl_{(C≤8)};
   R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamino_{(C≤12)}, acyl_{(C≤12)}, acyloxy_{(C≤12)}, amido_{(C≤12)}, or a substituted version thereof; or either two R₃ groups and R₂ and R₃ are taken together to form one or more cycloalkanediyl_{(C≤18)}, arenediyl_{(C≤18)}, heteroarenediyl_{(C≤18)}, heterocycloalkanediyl_{(C≤18)}, or a substituted version thereof; or a group of the formula: —AR₃′, wherein A is alkanediyl_{(C≤8)} or substituted alkanediyl_{(C≤8)}; and R₃′ is NR_{d}C(X₄)NR_{d}′R_{d}″, wherein X₄ is O or NRₑ, wherein Rₑ is hydrogen, alkyl_{(C≤6)}, or substituted alkyl_{(C≤6)}, and R_{d}, R_{d}′, and R_{d}″ are each independently hydrogen, alkyl_{(C≤6)}, or substituted alkyl_{(C≤6)}; and
   n is 1 or 2; provided that for each n, then each R₃ is separately selected;
provided that the compound is not eributin;
or a pharmaceutically acceptable salt thereof.

In some embodiments, the compounds are further defined as:

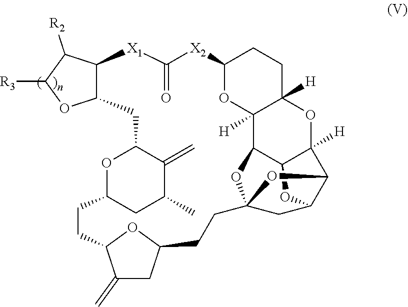

wherein:
   X₁ and X₂ is a covalent bond, alkanediyl_{(C≤8)}, substituted alkanediyl_{(C≤8)}, NRₐ, O, and
   S; wherein:
      Rₐ is hydrogen, alkyl_{(C≤8)}, or substituted alkyl_{(C≤8)};
   R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamino_{(C≤12)}, acyl_{(C≤12)}, acyloxy_{(C≤12)}, amido_{(C≤12)}, or a substituted version thereof; or either two R₃ groups and R₂ and R₃ are taken together to form one or more cycloalkanediyl_{(C≤18)}, arenediyl_{(C≤18)}, heteroarenediyl_{(C≤18)}, heterocycloalkanediyl_{(C≤18)}, or a substituted version thereof; or a group of the formula: —AR₃′, wherein A is alkanediyl_{(C≤8)} or substituted alkanediyl_{(C≤8)}; and R₃′ is NR_{d}C(X₄)NR_{d}′R_{d}″, wherein X₄ is O or NRₑ, wherein Rₑ is hydrogen, alkyl_{(C≤6)}, or substituted alkyl_{(C≤6)}, and R_{d}, R_{d}′ and R_{d}″ are each independently hydrogen, alkyl_{(C≤6)}, or substituted alkyl_{(C≤6)}; and
   n is 1 or 2; provided that for each n, then each R₃ is separately selected;
provided that the compound is not eribtutin;
or a pharmaceutically acceptable salt thereof.

In some embodiments, Y₂ is CR_{b}R_{b}′. In some embodiments, R_{b} is hydrogen. In some embodiments, R_{b}′ is hydrogen. In some embodiments, Y₃ is CR_{b}R_{b}′. In some embodiments, R_{b} is hydrogen. In some embodiments, R_{b}′ is hydrogen.

In some embodiments, Y₁ is O. In some embodiments, X₃ is O. In some embodiments, x is 1. In some embodiments, y is 1. In some embodiments, z is 1. In some embodiments, R₁ is alkyl_{(C≤8)} or substituted alkyl_{(C≤8)}. In some embodiments, R₁ is alkyl_{(C≤8)} such as methyl. In some embodiments, R₂ is hydroxy. In other embodiments, R₂ is alkyl_{(C≤8)} or substituted alkyl_{(C≤8)}. In some embodiments, R₂ is alkyl_{(C≤8)} such as methyl. In other embodiments, R₂ is alkoxy_{(C≤8)} or substituted alkoxy_{(C≤8)}. In some embodiments, R₂ is alkoxy_{(C≤8)} such as methoxy.

In some embodiments, R₃ is hydroxy. In other embodiments, R₃ is alkyl_{(C≤8)} or substituted alkyl_{(C≤8)}. In some embodiments, R₃ is alkyl_{(C≤8)} such as methyl. In other embodiments, R₃ is substituted alkyl_{(C≤8)}, such as hydroxymethyl, hydroxypropyl, or dihydroxypropyl. In other embodiments, R₃ is alkoxy_{(C≤8)}, or substituted alkoxy_{(C≤8)}. In some embodiments, R₃ is alkoxy_{(C≤8)} such as methoxy.

In other embodiments, R₃ is a group of the formula: —AR₃′, wherein A is alkanediyl_{(C≤8)} or substituted alkanediyl_{(C≤8)}; and R₃ is NR_{d}C(X₄)NR_{d}′R_{d}″, wherein X₄ is O or NRₑ, wherein Rₑ is hydrogen, alkyl_{(C≤6)}, or substituted alkyl_{(C≤6)}, and R_{d}, R_{d}′, and R_{d}″ are each independently hydrogen, alkyl_{(C≤6)}, or substituted alkyl_{(C≤6)}. In some embodiments, A is substituted alkanediyl_{(C≤8)}. In some embodiments, A is a hydroxy substituted propanediyl. In some embodiments, R₃′ is NR_{d}C(X₄)NR_{d}′R_{d}″. In some embodiments, X₄ is O. In some embodiments, R_{d}″ is hydrogen. In some embodiments, R_{d}′ is hydrogen. In some embodiments, R″ is hydrogen. In other embodiments, R₃ and R₂ are taken together and are heterocycloalkanediyl_{(C≤18)} or substituted heterocycloalkanediyl_{(C≤18)}.

In some embodiments, the compounds are further defined as:

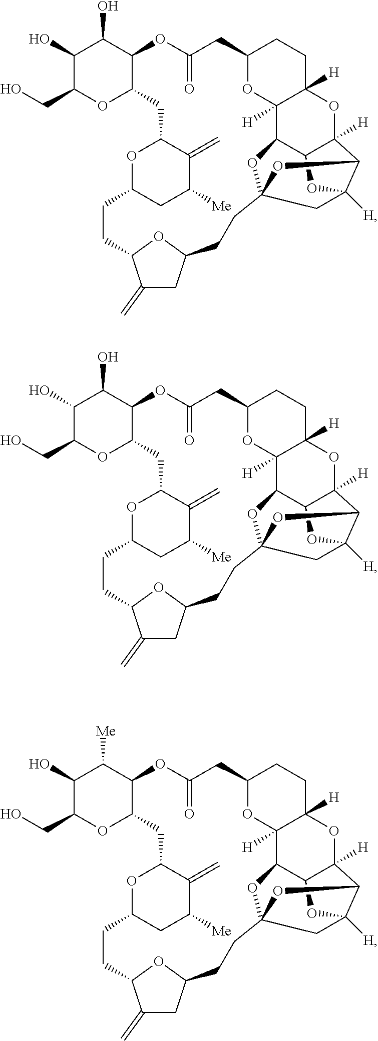

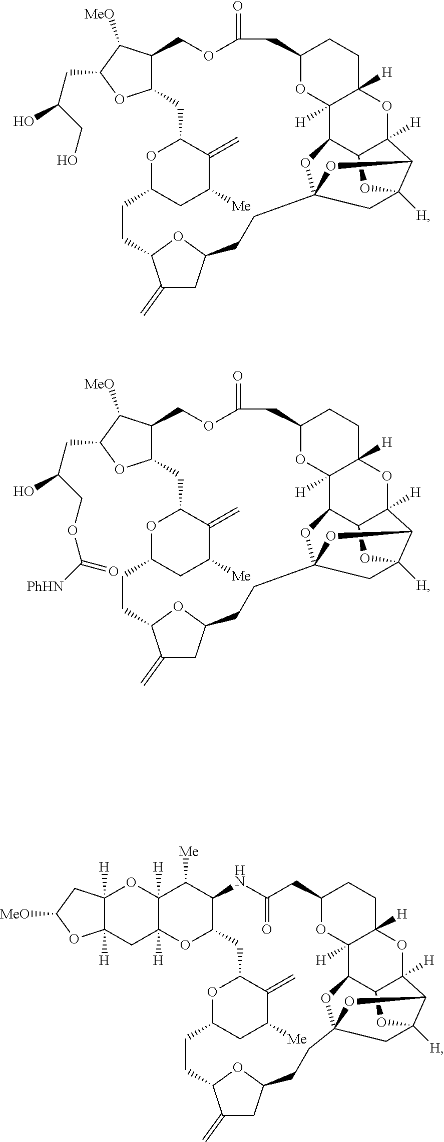

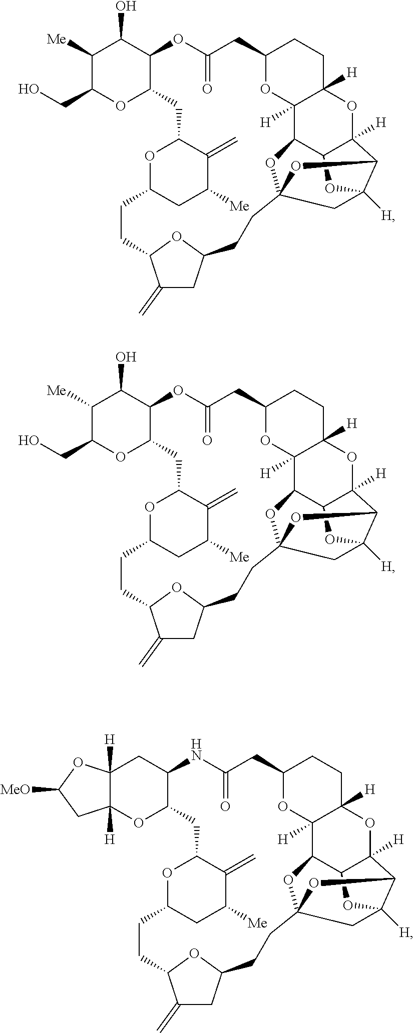

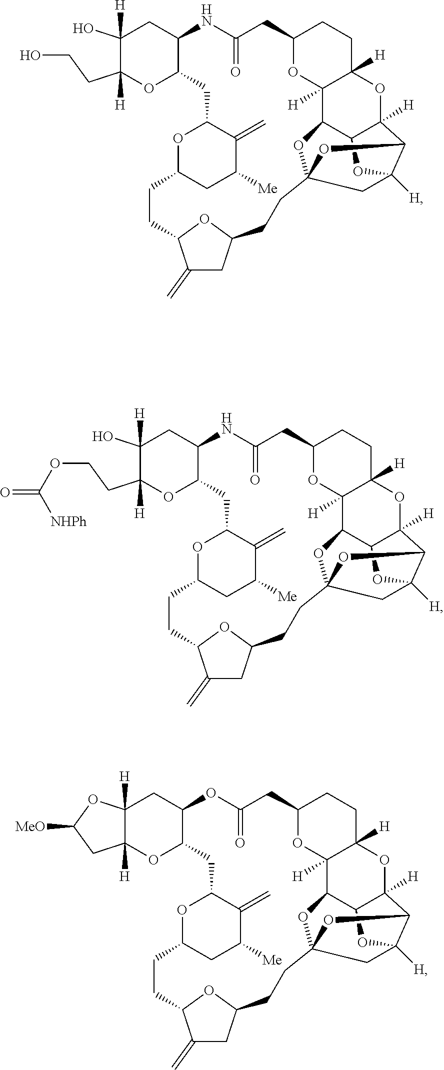

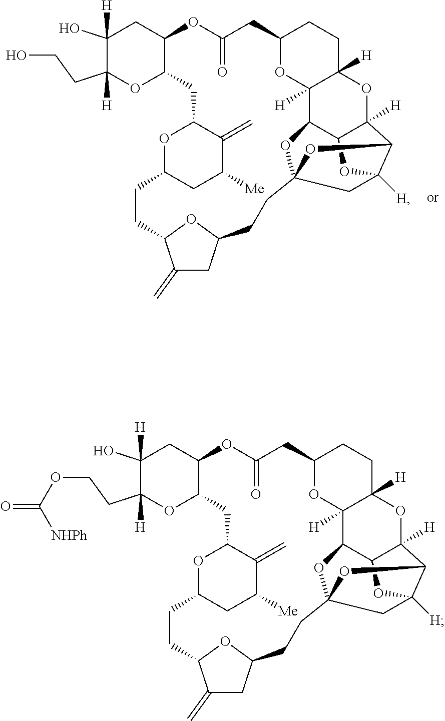

or a pharmaceutically acceptable salt thereof.

In another aspect, the present disclosure provides pharmaceutical compositions comprising a compound described herein and an excipient. In some embodiments, the compositions are formulated for oral administration, administration by injection, or topical administration.

In still another aspect, the present disclosure provides methods of treating a disease or disorder in a patient in need thereof comprising administering to the patient a therapeutically effective amount of a compound or pharmaceutically composition described herein. In some embodiments, the disease or disorder is cancer. In some embodiments, the disease or disorder is a carcinoma, sarcoma, lymphoma, leukemia, melanoma, mesothelioma, multiple myeloma, or seminoma. In some embodiments, the disease or disorder is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the methods further comprise administering a second therapeutic agent such as a second chemotherapeutic agent, surgery, photodynamic therapy, sonodynamic therapy, radiotherapy, or immunotherapy.

In still yet aspect, the present disclosure provides methods of making erbulin or a compound of claim 1 comprising reacting an intermediate of the formula:

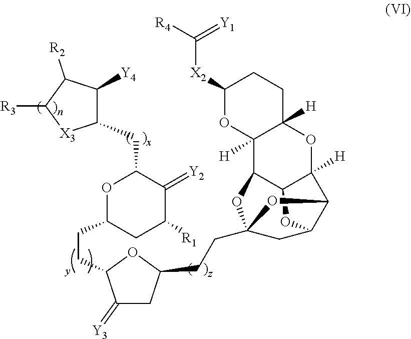

wherein:
   Y₁, Y₂, Y₃, X₂, X₃, R₁, R₂, R₃, n, x, y, and z are as defined above;
   Y₄ is halo, specifically iodo; and
   R₄ is hydrogen, alkoxy_{(C≤12)}, or substituted alkoxy_{(C≤12)};
with a transition metal catalyst to obtain the erbulin or compound.

In still yet another aspect, the present disclosure provides intermediates of the formula:

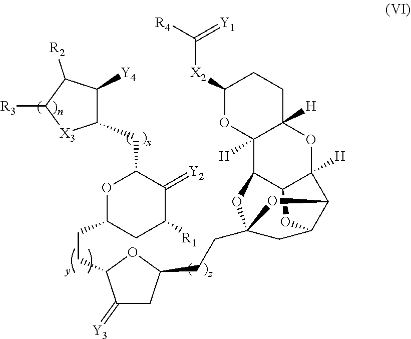

wherein.
   X₁, X₂, and X₃ are each independently a covalent bond, alkanediyl_{(C≤8)}, substituted alkanediyl_{(C≤8)}, NRₐ, O, and S; wherein:
      Rₐ is hydrogen, alkyl_{(C≤8)}, or substituted alkyl_{(C≤8)};
   Y₁, Y₂, and Y₃ are each independently CR_{b}R_{b}′, O, S, or NR_{c}, wherein
      R_{b}, R_{b}′, and R_{c} are each independently hydrogen, alkyl_{(C≤8)}, or substituted alkyl_{(C≤8)};
   x, y, and z are each independently 1, 2, or 3;
   R₁ is hydrogen, alkyl_{(C≤8)}, or substituted alkyl_{(C≤8)};
   R₂ and R₃ are each independently alkyl_{(C≤12)}, alkenyl_{(C≤12)}, alkynyl_{(C≤12)}, alkoxyt_{(C≤12)}, alkylamino_{(C≤12)}, dialkylamino_{(C≤12)}, acyl_{(C≤12)}, acyloxy_{(C≤12)}, amido_{(C≤12)}, or a substituted version thereof; or either two R₃ groups and R₂ and R₃ are taken together to form one or more cycloalkanediyl_{(C≤18)}, arenediyl_{(C≤18)}, heteroarenediyl_{(C≤18)}, heterocycloalkanediyl_{(C≤18)}, or a substituted version thereof; or a group of the formula: —AR₃′, wherein A is alkanediyl_{(C≤8)} or substituted alkanediyl_{(C≤8)}; and R₃′ is NR_{d}C(X₄)NR_{d}′R_{d}″, wherein X₄ is O or NRₑ, wherein Rₑ is hydrogen, alkyl_{(C≤8)}, or substituted alkyl_{(C≤8)}, and R_{d}, R_{d}′, and R_{d}″ are each independently hydrogen, alkyl_{(C≤8)}, or substituted alkyl_{(C≤8)};
   n is 1 or 2; provided that for each n, then each R₃ is separately selected;
   Y₄ is halo, specifically iodo; and
   R₄ is hydrogen, alkoxy_{(C≤12)}, or substituted alkoxy_{(C≤12)}.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### DESCRIPTION OF ILLUSTRATIVE EMBODIMENTS

In some aspects, the present disclosure provides halichondrins analogs that may be used in the treatment of cancer. These compounds show improved properties relative to erbutin and other halichondrins analogs. These and more details are set out below.

### I. Compounds and Formulations Thereof

### A. Compounds

The compounds provided by the present disclosure are shown, for example, above in the summary section and in the examples and claims below. They may be made using the methods outlined in the Examples section. The analogs described herein can be synthesized according to the methods described, for example, in the Examples section below. These methods can be further modified and optimized using the principles and techniques of organic chemistry as applied by a person skilled in the art. Such principles and techniques are taught, for example, in March's Advanced Organic Chemistry: Reactions, Mechanisnis, and Structure (2007), which is incorporated by reference herein.

The analogs described herein may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the (S) or the (R) configuration.

Chemical formulas used to represent the analogs described herein will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

The analogs described herein may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the analogs described herein are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The analogs described herein may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the diselsoure contemplates prodrugs of compounds of the present disclosure as well as methods of delivering prodrugs. Prodrugs of the analogs described herein may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

Those skilled in the art of organic chemistry will appreciate that many organic compounds can form complexes with solvents in which they are reacted or from which they are precipitated or crystallized. These complexes are known as “solvates.” For example, a complex with water is known as a “hydrate.” Solvates of the analogs described herein are within the scope of the discisoure. It will also be appreciated by those skilled in organic chemistry that many organic compounds can exist in more than one crystalline form. For example, crystalline form may vary from solvate to solvate. Thus, all crystalline forms of the analogs described herein are within the scope of the present disclosure.

### B. Formulations

In some embodiments of the present disclosure, the analogs are included a pharmaceutical formulation. Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., the tubulysin analogs described herein) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, nagnesiun aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

### II. Cancer and Other Hyperproliferative Diseases

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, the analogs described herein may be used to lead to decreased cell counts and as such can potentially be used to treat a variety of types of cancer lines. In some aspects, it is anticipated that the analogs described herein may be used to treat virtually any malignancy.

Cancer cells that may be treated with the compounds of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidernoid carcinoma; cystadenocarcinoma; papillary-cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastomna; hepatoblastoma; carcinosarcoma; mesenchymoma, malignant; Brenner tumor, malignant; phyllodes tumor, malignant; synovial sarcoma; mesothelioma, malignant; dysgerninoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignanthistiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; mycloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### III. Cell Targeting Moieties

In some aspects, the present disclosure provides compounds conjugated directly or through linkers to a cell targeting moiety. In some embodiments, the conjugation of the compound to a cell targeting moiety increases the efficacy of the compound in treating a disease or disorder. Cell targeting moieties according to the embodiments may be, for example, an antibody, a growth factor, a hormone, a peptide, an aptamer, a small molecule such as a hormone, an imaging agent, or cofactor, or a cytokine. For instance, a cell targeting moiety according the embodiments may bind to a liver cancer cell such as a Hep3B cell. It has been demonstrated that the gp240 antigen is expressed in a variety of melanomas but not in normal tissues. Thus, in some embodiments, the compounds of the present disclosure may be used in conjugates with an antibody for a specific antigen that is expressed by a cancer cell but not in normal tissues.

In certain additional embodiments, it is envisioned that cancer cell targeting moieties bind to multiple types of cancer cells. For example, the 8H9 monoclonal antibody and the single chain antibodies derived therefrom bind to a glycoprotein that is expressed on breast cancers, sarcomas and neuroblastomas (Onda, et al., 2004). Another example is the cell targeting agents described in U.S. Patent Publication No. 2004/005647 and in Winthrop, et al. (2003) that bind to MUC-1, an antigen that is expressed on a variety cancer types. Thus, it will be understood that in certain embodiments, cell targeting constructs according the embodiments may be targeted against a plurality of cancer or tumor types.

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the cell-specific targeting moieties in cancer therapy. Additionally, the cell targeting moiety that may be used include a cofactor, a sugar, a drug molecule, an imaging agent, or a fluorescent dye. Many cancerous cells are known to over express folate receptors and thus folic acid or other folate derivatives may be used as conjugates to trigger cell-specific interaction between the conjugates of the present disclosure and a cell (Campbell, et al., 1991; Weitman, et al., 1992).

Since a large number of cell surface receptors have been identified in hematopoietic cells of various lineages, ligands or antibodies specific for these receptors may be used as cell-specific targeting moieties. IL-2 may also be used as a cell-specific targeting moiety in a chimeric protein to target IL-2R-cells. Alternatively, other molecules such as B7-1, B7-2 and CD40 may be used to specifically target activated T cells (The Leucocyte Antigen Facts Book, 1993, Barclay, et al. (eds.), Academic Press). Furthermore, B cells express CD19, CD40 and IL-4 receptor and may be targeted by moieties that bind these receptors, such as CD40 ligand, IL-4, IL-5, IL-6 and CD28. The elimination of immune cells such as T cells and B cells is particularly useful in the treatment of lymphoid tumors.

Other cytokines that may be used to target specific cell subsets include the interleukins (IL-I through IL-15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego). In some aspects, the targeting polypeptide is a cytokine that binds to the Fn14 receptor, such as TWEAK (see, e.g., Winkles, 2008; Zhou, et al., 2011 and Burkly, et al., 2007, incorporated herein by reference).

A skilled artisan recognizes that there are a variety of known cytokines, including hematopoietins (four-helix bundles) [such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)]; interferons [such as IFN-γ, IFN-α, and IFN-β); immunoglobin superfamily (such as B7.1 (CD80), and B7.2 (B70, CD86)]; TNF family [such as TNF-α (cachectin), TNF-β (lymphotoxin, LT, LT-α), LT-β, CD40 ligand (CD40L), Fas ligand (FasL), CD27 ligand (CD27L), CD30 ligand (CD30L), and 4-IBBL)]; and those unassigned to a particular family [such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)]. Furthermore, the Fe portion of the heavy chain of an antibody may be used to target Fe receptor-expressing cells such as the use of the Fe portion of an IgE antibody to target mast cells and basophils.

Furthermore, in some aspects, the cell-targeting moiety may be a peptide sequence or a cyclic peptide. Examples, cell- and tissue-targeting peptides that may be used according to the embodiments are provided, for instance, in U.S. Pat. Nos. 6,232,287; 6,528,481; 7,452,964; 7,671,010; 7,⁷81,565; 8,507,445; and 8,450,278, each of which is incorporated herein by reference.

Thus, in some embodiments, cell targeting moieties are antibodies or avimers. Antibodies and avimers can be generated against virtually any cell surface markerthus, providing a method fortargeted to delivery of OrB to virtually any cell population of interest. Methods for generating antibodies that may be used as cell targeting moieties are detailed below. Methods for generating avimers that bind to a given cell surface marker are detailed in U.S. Patent Publications Nos. 2006/0234299 and 2006/0223114, each incorporated herein by reference.

Additionally, it is contemplated that the compounds described herein may be conjugated to a nanoparticle or other nanomaterial. Some non-limiting examples of nanoparticles include metal nanoparticles such as gold or silver nanoparticles or polymeric nanoparticles such as poly-L-lactic acid or poly(ethylene) glycol polymers. Nanoparticles and nanomaterials which may be conjugated to the instant compounds include those described in U.S. Patent Publications Nos. 2006/0034925, 2006/0115537, 2007/0148095, 2012/0141550, 2013/0138032, and 2014/0024610 and PCT Publication No. 2008/121949, 2011/053435, and 2014/087413, each incorporated herein by reference.

### IV. Therapies

### A. Pharmaceutical Formulations and Routes of Administration

Where clinical applications are contemplated, it will be necessary to prepare pharmaceutical compositions in a form appropriate for the intended application. In some embodiments, such formulation with the tubulysin analogs of the present disclosure is contemplated. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils, tinder ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable wider the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the tubulysin analogs described herein may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts forned with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 mL of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the appropriate regulatory agencies for the safety of pharmaceutical agents.

### B. Methods of Treatment

In particular, the compositions that may be used in treating cancer in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that induces death of cancerous cells (e.g., induces apoptosis of a cancer cell), as assayed by identifying a reduction in hematological parameters (complete blood count—CBC), or cancer cell growth or proliferation. In some embodiments, amounts of the tubulysin analogs used to induce apoptosis of the cancer cells is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the tubulysin analogs may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer in which the subject has been administered a therapeutic amount of a composition as described herein. The level of marker determined in the method can be compared to known levels of marker in either healthy normal controls or in other afflicted patients to establish the subject's disease status. In preferred embodiments, a second level of marker in the subject is determined at a time point later than the detennination of the first level, and the two levels are compared to monitor the course of disease or the efficacy ofthe therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

### C. Combination Therapies

It is envisioned that the analogs described herein may be used in combination therapies with one or more cancer therapies or a compound which mitigates one or more of the side effects experienced by the patient. It is common in the field of cancer therapy to combine therapeutic modalities. The following is a general discussion of therapies that may be used in conjunction with the therapies of the present disclosure.

To treat cancers using the methods and compositions of the present disclosure, one would generally contact a tumor cell or subject with a compound and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the analogs described herein may precede or follow the other treatment by intervals ranging from minutes to weeks. One would generally ensure that a significant period of time did not expire between the times of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. The following is a general discussion of cancer therapies that may be used combination with the compounds of the present disclosure.

### 1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues. KW-2189 and CB1-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ₁ and calicheamicin co; dynemicin, including dynemicin A; uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansanitocins; mitoguazone; mitoxantrone; mopidammol: nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichloro-triethylamine; trichothecenes (especially T-2 toxin, verracurin A, roridin A and anguidine); urethan; vindesine: dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone: vincristine; vinorelbine; novantrone; teniposide; edatrexate; daunomycin; aminopterin; xeloda; ibandronate; irinotecan (e.g., CPT-11); topoisomerase inhibitor RFS 2000; difluorometihylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

### 2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermiia, the use of heat, is also being studied for its effectiveness in sensitizing tissue to radiation.

### 3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MTP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Pliasnodiunm falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF and TNF (Bukowski, et al., 1998; Davidson, et a., 1998; Hellstrand, et al., 1998) gene therapy, e.g., TNF, TL-1, IL-2, p53 (Qin et a., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras, et al., 1998; Hanibuchi, et a, 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al, 1993).

In adoptive immunotherapy, the patientfs circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

### 4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believe to be particularly efficacious in reducing the reoccurrence of the tumor. Additionally, the compounds of the present disclosure can also be used in a neoadjuvant setting.

### 5. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents may be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 41.1° C.). External or internal heating devices may be involved in the application of local, regional, or whole-body hyperthennia. Local hyperthennia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the appropriate pharmaceutical agent regulatory agencies.

It also should be pointed out that any of the foregoing therapies may prove useful by themselves in treating cancer.

### V. Synthetic Methods

In some aspects, the analogs of this discisoure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

### A. Process Scale-Up

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research &f Development (2000), which is incorporated by reference herein. The synthetic method described herein may be used to produce preparative scale amounts of the tubulysin analogs described herein.

### B. Chemical Definitions

When used in the context of a chemical group: “hydrogen” means —H; “hydroxy” means —OH; “oxo” means=O: “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imuno means=NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof: “mercapto” means —SH; and “thio” means=S; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

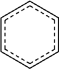

covers, for example,

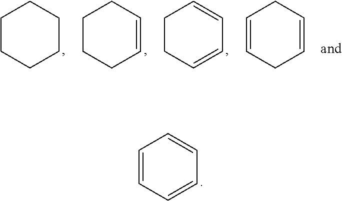

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

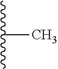

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of” the page. The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

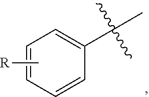

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

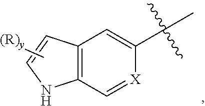

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefint_{(C5)}”, and “olefin_{C5}” are all synonymous. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic 21 system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃(tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)CH₂—, and —CH₂CH₂CH₂— are non-limiting examples of alkanediyl groups.

The term “alkylidene” when used without the “substituted” modifier refers to the divalent group==CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these tenns is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃. —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CHOCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

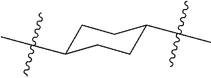 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C—CH, —C—CCH₃, and —CH₂C—CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO?, —CO₂H. —CO₂CH₃, —CN, —SH, —OCH₃, —OCH₂CH₃, —C(O)CH₃, —NHCH₃, —NHCH₂CH, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

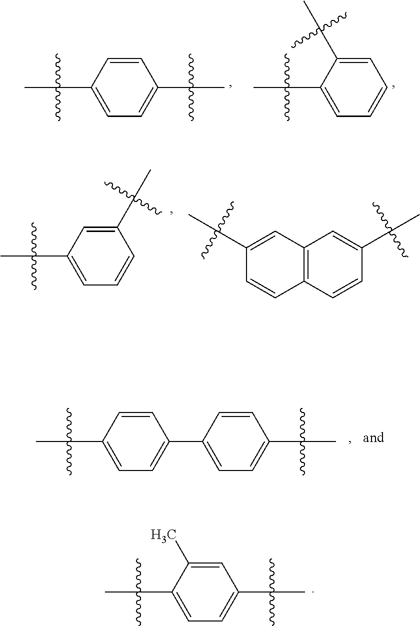

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH, —NO, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₂, —NHC(O)CH₃, —S(O)₂OH, or —S(O)₂NH₂. Non-limiting examples of substituted aralkyls are: (3-chlorophenyl)-methyl, and 2-chloro-2-phenyl-eth-_-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl) —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH, —OCH(CH₃)₂(isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR— in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, where in at least one of the hydrogen atoms has been replaced with an alkoxy group. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCHCH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifi^{e}r refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”. “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. When any of these terms is used with the “substituted” modifier, one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃), —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

As indicated above in some aspects the cell-targeting moiety is an antibody. As used herein, the term “antibody” is intended to include immunoglobulins and fragments thereof which are specifically reactive to the designated protein or peptide, or fragments thereof. Suitable antibodies include, but are not limited to, human antibodies, primatized antibodies, de-immunized antibodies, chimeric antibodies, bi-specific antibodies, humanized antibodies, conjugated antibodies (i.e., antibodies conjugated or fused to other proteins, radiolabels, cytotoxins), Small Modular ImmnnoPharmaceuticals (“SMIPs™”), single chain antibodies, cameloid antibodies, antibody-like molecules (e.g., anticalins), and antibody fragments. As used herein, the term “antibodies” also includes intact monoclonal antibodies, polyclonal antibodies, single domain antibodies [e.g., shark single domain antibodies (e.g., IgNAR or fragments thereof)], multispecific antibodies (e.g., bi-specific antibodies) forned from at least two intact antibodies, and antibody fragments so long as they exhibit the desired biological activity. Antibody polypeptides for use herein may be of any type (e.g., IgG, TgM, IgA, IgD and IgE). Generally, IgG and/or IgM are preferred because they are the most common antibodies in the physiological situation and because they are most easily made in a laboratory setting. As used herein the term antibody also encompasses an antibody fragment such as a portion of an intact antibody, such as, for example, the antigen-binding or variable region of an antibody. Examples of antibody fragments include Fab, Fab′, F(ab″)₂, Fe and Fv fragments; triabodies; tetrabodies; linear antibodies; single-chain antibody molecules; and multi specific antibodies formed from antibody fragments. The term “antibody fragment” also includes any synthetic or genetically engineered protein that acts like an antibody by binding to a specific antigen to form a complex. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. An oxygen linked antibody is an antibody which has a chemical function group such that the linkage between the antibody and the linker or compound is joined via an oxygen atom. Similarly, a nitrogen linked antibody is an antibody which has a chemical function group such that the linkage between the antibody and the linker or compound is joined via a nitrogen atom.

A “linker” in the context of this application is divalent chemical group which may be used to join one or more molecules to the compound of the instant disclosure. Linkers may also be an amino acid chain wherein the carboxy and amino terminus serve as the points of attachment for the linker. In some embodiments, the linker contains a reactive functional group, such as a carboxyl, an amide, an amine, a hydroxy, a mercapto, an aldehyde, or a ketone on each end that be used to join one or more molecules to the compounds of the instant disclosure. In some non-limiting examples, —CH₂CH₂CH₂CH₂—, —C(O)CH₂CH₂CH₂—, —OCH₂CH₂NH—, NHCH₂CH₂NH—, and —(OCH₂CH₂)ₙ—, wherein n is between 1-1000, are linkers.

An “amine protecting group” or “amino protecting group” is well understood in the art. An amine protecting group is a group which prevents the reactivity of the amine group during a reaction which modifies some other portion of the molecule and can be easily removed to generate the desired amine. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chiorobenzyioxycarbonyl, p-methoxybenzyloxycarbonyl, p-nitrobenzyloxycarbonyl, 2-nitrobenzyioxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethyl-silylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymnethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defimed above. In some embodiments, the halogenated phthalimide derivative may be tetrachlorophthalimide (TCphth). When used herein, a “protected amino group”, is a group of the formula PG_{MA}NH— or PG_{DA}N-wherein PG_{MA} is a monovalent amine protecting group, which may also be described as a “monvalently protected amino group” and PGDA is a divalent amine protecting group as described above, which may also be described as a “divalently protected amino group”.

A “hydroxyl protecting group” or “hydroxy protecting group” is well understood in the art. A hydroxyl protecting group is a group which prevents the reactivity of the hydroxyl group during a reaction which modifies some other portion of the molecule and can be easily removed to generate the desired hydroxyl. Hydroxyl protecting groups can be found at least in Greene and Wuts, 1999, which is incorporated herein by reference. Some non-limiting examples of hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, a-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, P-bromobenzyloxycarbonyl, 3,4-dimethoxybenzyloxycarbonyl, 3,5-dimethoxybenzyloxycarbonyl, 2,4-dimethoxybenzyioxycarbonyl, 4-methoxybenzyioxycarbonyl, 2-nitro-4,5-dimethoxybenzyloxycarbonyl, 3,4,5-trimethoxybenzyioxycarbonyl, 1-(p-biphenylyl)-1-methyl-ethoxycarbonyl,α,α-dimethyl3,5-dimethoxybenzyloxycarbonyl, benzhydryloxycarbonyl, t-butyloxy-carbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. When used herein, a protected hydroxy group is a group of the fonnula PG_{H}O— wherein PG_{H} is a hydroxyl protecting group as described above.

A “thiol protecting group” is well understood in the art. A thiol protecting group is a group which prevents the reactivity of the mercapto group during a reaction which modifies some other portion of the molecule and can be easily removed to generate the desired mercapto group. Thiol protecting groups can be found at least in Greene and Wuts, 1999, which is incorporated herein by reference. Some non-limiting examples of thiol protecting groups include acyl groups such as fornyl, acetyl, propionyl, pivaloyl, t-butylacetyl, 2-chloroacetyl, 2-bromoacetyl, trifluoroacetyl, trichloroacetyl, o-nitrophenoxyacetyl, a-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trmethylsilylethyloxycarbonyl (Teoc), phenoxycarbonyl, 4-nitrophenoxycarbonyl, fluorenyl-9-methoxycarbonyl (Fmoc), cyclopentyloxy-carbonyl, adamantyloxycarbonyl, cyclohexyloxycarbonyl, phenylthiocarbonyl and the like; aralkyl groups such as benzyl, triphenyliethyl, benzyloxymethyl and the like; and silyl groups such as trimethylsilyl and the like. When used herein, a protected thiol group is a group of the formula PG_{T}S—wherein PG_{T} is a thiol protecting group as described above.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedrally substituted carbon centers), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its (R) form. (S) form, or as a mixture of the (R) and (S) forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

### VI. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### I. Improved Synthesis of Fragment MN (17) and Further Optimization of Its Conversion to Fragment IJKLMN (8).

Scheme 1 summarizes the synthesis of required fragment MN(17) starting with our previously reported building blocks 9 and 10 through the intermediacy of aldehyde 11.⁶ Instead of the previously used tin-mediated radical cyclization,⁶ a nickel-catalyzed ynal reductive cyclization⁸ was employed to construct the bicyclic ring system with improved yield. Thus, aldehyde 11 was subjected to Ni(cod)₂/n-Bu₃P/Et₃SiH conditions,⁹ leading to transient intermediate 12, which was selectively deprotected (0.5 M aq. HCl) to afford allylic alcohol 13 in 83% yield (as compared to 53% yield with tin reductive conditions).⁶ The excellent diastereoselectivity (>15:1) in forming bicyclic intermediate 13 indicated that the nickel-catalyzed ynal reductive cyclization may proceed through a preferred chair transition state 12a, as shown in Scheme 1. It should be noted that other conditions such as SmI₂, Cp₂TiCl,¹⁰ and Cp₂Ti(PMe₃)₂,¹¹ PtCl₂/P(p-CF₃C6H₄)₃¹² and VCl₃(THF)₃/Zn¹³ either led to low yield or failed to produce the desired product (13). Treatment of alcohol 13 with Dess-Martin reagent afforded enone 14, whose olefinic bond was oxidatively cleaved (O₃, MeOH; then NaBH₄) to furnish, directly and stereoselectively,¹⁴ diol 15 in 65% overall yield for the two steps, as shown in Scheme 1. One-pot acetonide formation and desilylation of the latter (12 M aq. HCl, acetone:MeOH, 77% yield) followed by Dess-Martin oxidation (DMP) of the resulting primary alcohol led to the desired fragment MN(17). The current eight-step sequence to 17 in 14% overall yield from building block 10 represents a significant improvement from our previous nine-step sequence (6.3% overall yield).⁶

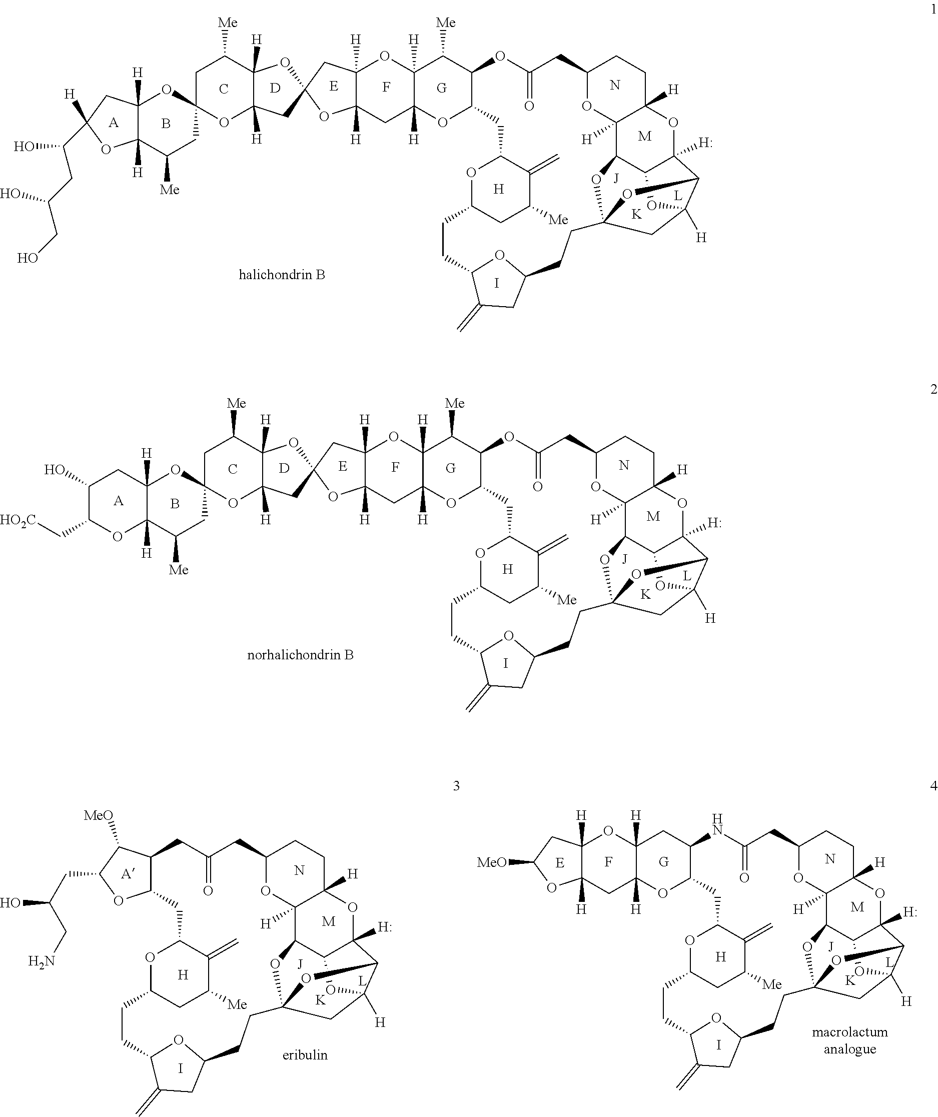

Molecular structures of halichondrin B (1), norhalichondrin B (2), eribulin (3), and macrolactam analogue 4.

Having improved the synthetic route to fragment Mu(17), attention was turned to optimizing the benzyl cleavage from diol 19, which was achieved in two steps and 78% overall yield⁶ as shown in Scheme 2. After extensive experimentation, a continuous flow process was developed to achieve the sought-after improvement. Thus, exposure of a mixture of a mixture of 19 (2:1 dr at C12), DDQ, and BT to LED light (λ=390 nim)¹⁵ using a continuous flow process resulted in clean cleavage of the benzyl protecting group, affording the corresponding triol intermediate as a mixture of C12-epimers (20 and C12-epi-20, 2:1 dr). The latter mixture was then exposed to intramolecular ketalization conditions (p-TsOH·H₂O) affording cage compound 21 (70%) yield from 19); and leaving behind C12-epi-20 (67% from C12-epi-19) which was isolated and recycled through epimerization at C12 (NaOMe) to 20 (90% yield, 2.2:1 dr), thus further enriching the material supply of 21. Finally, the desired mesylate 8 (fragment IJKLMN) was prepared (MsCl, Et₃N, 95% yield) as previously reported⁶ and shown in Scheme 2.

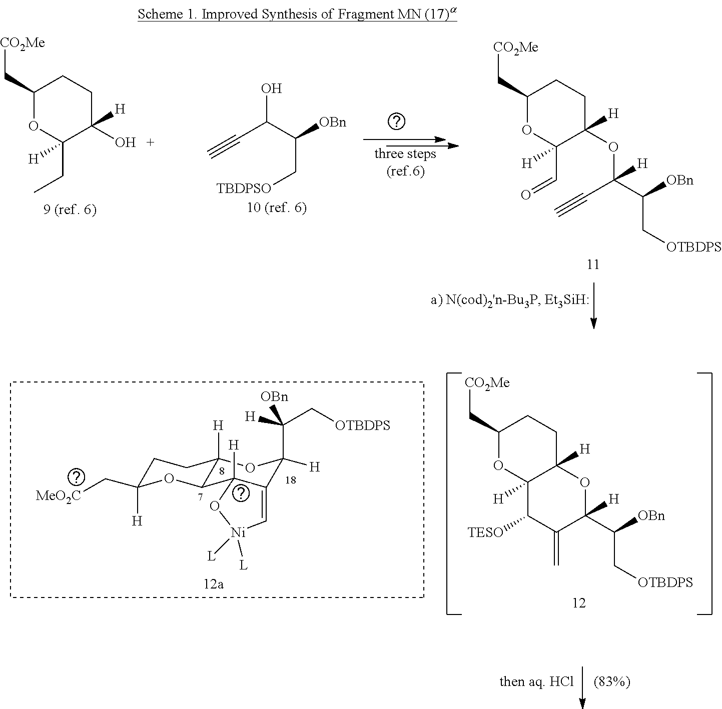

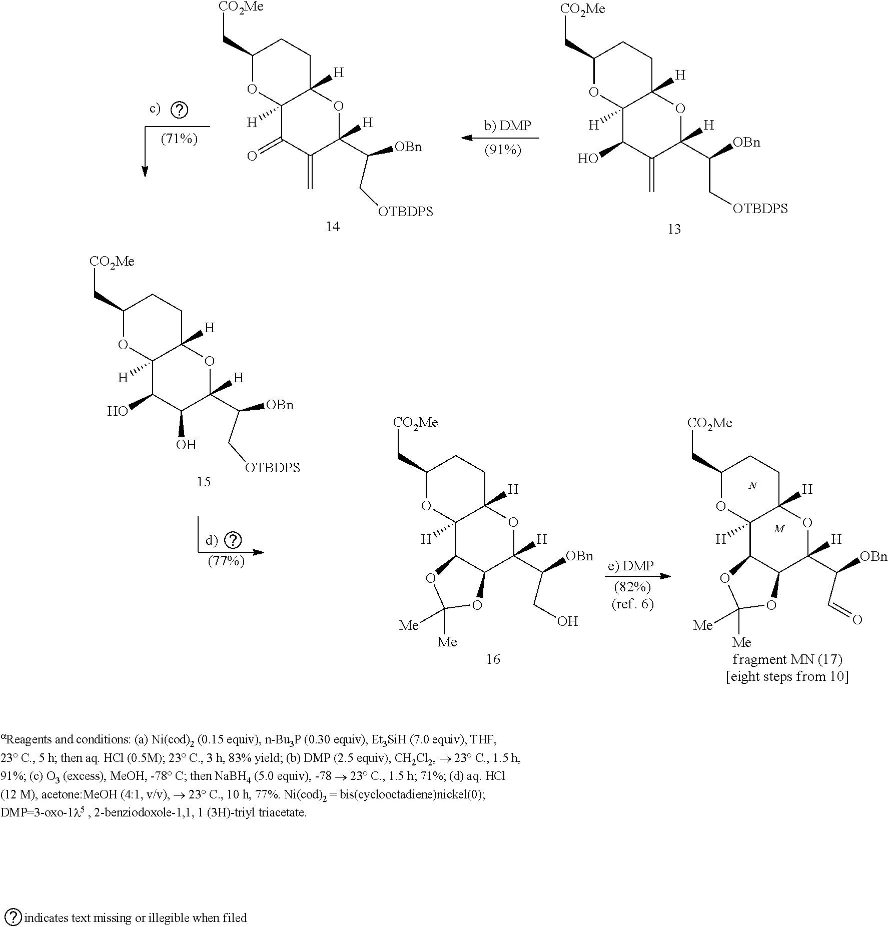

### II. First Generation Synthesis of Fragment A′ (33) and Efforts toward Eribulin (3)

The first attempt to synthesize eribulin (3) required, in addition to fragment IJKLMN (8), tosylsulfone aldehyde fragment A′ (33, see Scheme 3). The synthesis of tetrahydrofuran aldehyde fragment A′ (33) started from readily available building blocks α-hydroxy ester 22¹⁶ and hydroxy acetylene 23⁶ as summarized in Scheme 3. Thus, Nicholas etherification of 22 and 23 [Co₂(CO₈); then BF₃·Et₂O; then (NH)₄Ce(NO₃)₆] furnished linear ethers 24a (31% yield) and 24b (46% yield) as a chromatographically separable mixture of diastereoisomers. The major and desired diastereoisomer 24b (whose correct configuration was confirmed at a later stage, after a number of steps, see below) was then converted to cyclization precursor 25 through sequential reductions of its alkyne (Lindlar catalyst, H₂) and ester (DIBAL-H) functionalities, respectively, in 88% overall yield, as shown in Scheme 3. Exposure of the latter to hydroxylamine hydrochloride followed by oxidation (NaOCl) of the resulting oxime furnished the corresponding nitrile oxide leading to spontaneous 1,3-dipolar cycloaddition (see dashed central box in Scheme 3 for a mechanistic rationale), affording isoxazoline 26 (62% overall yield). The stereochemical configuration of 26 was confirmed by NOE spectroscopic studies, which also confirmed the configurations of its precursors 24b and 25. Reductive N-0 bond cleavage within isoxazoline 26 [Mo(CO)₆, MeCN, 72% yield] followed by stereoselective reduction [Me₄NBH(OAc)₃, 95% yield] of the so-formed ketone 27 afforded diol 28 in 68% overall yield from 26. The latter diol was transformed to sulfone 30, in 81% overall yield, through a three-step sequence via intermediate 29 involving selective sulfurization (p-tolyl disulfide, n-Bu₃P, 90% yield) of the primary alcohol, methylation (NaH, MeI) of the secondary hydroxyl group, and oxidation (Oxone, 90% overall yield) of the resulting organosulfur derivative, as depicted in Scheme 3. Sulfone 30 was then subjected to desilylation (HF·py), and the resulting primary alcohol was reprotected with a pivaloyl group (PivCl, py) to afford olefin 31 (94% overall yield), whose Sharpless dihydroxylation¹⁷ (AD-mix-α, MeSO₂NH₂) led to the diastereoselective formation of the corresponding diol intermediate, which was silylated to form intermediate 32 in 70% overall yield as shown in Scheme 3. Selective cleavage of pivaloyl protecting group of 32 (NaOMe), followed by Dess-Martin oxidation of the resulting primary alcohol, furnished coveted sulfone-aldehyde fragment A′ (33), in 81% overall yield as shown in Scheme 3.

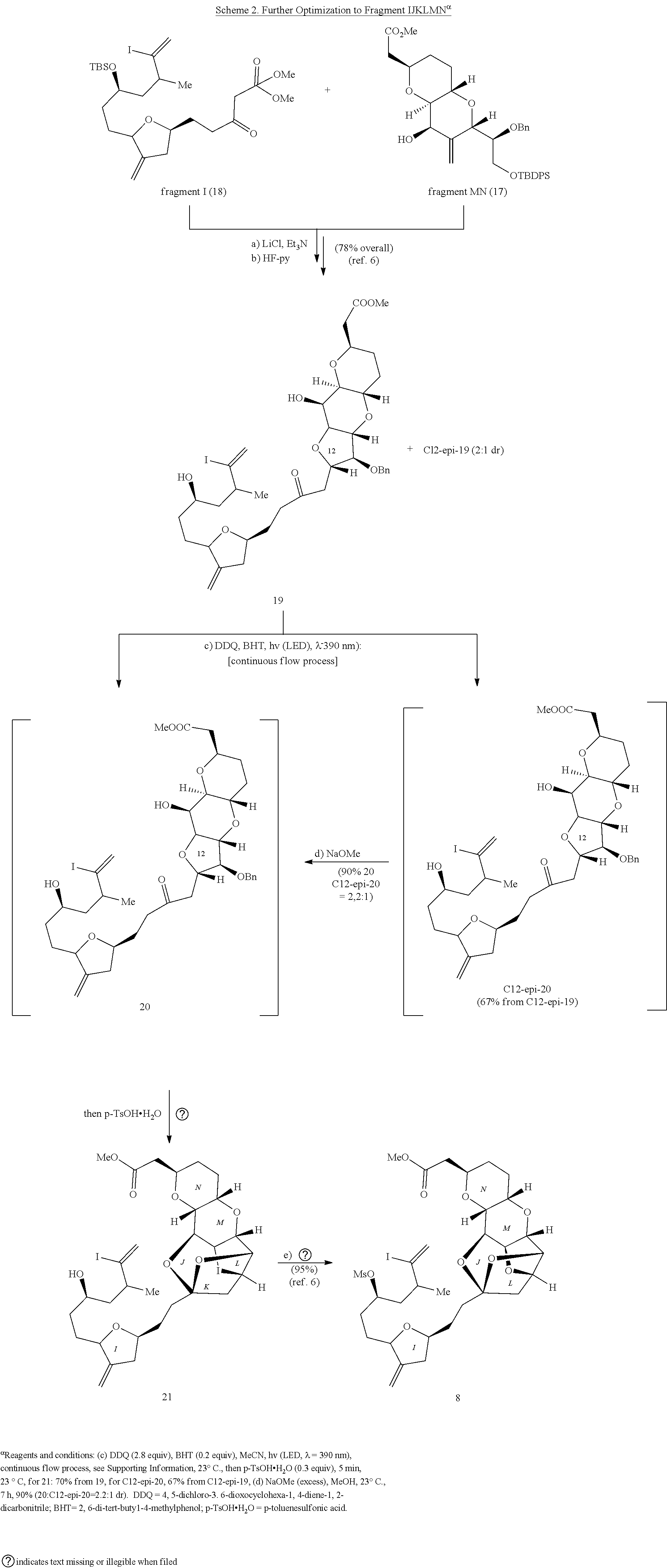

With both fragments A′ (33) and IJKLMN (8) readily available, the stage was set to their coupling and further elaboration toward the targeted eribulin (3). Thus, and as shown in Scheme 4, Nozaki-Hiyama-Kishi reaction^{2a} between 33 and 8 (NiCl₂/CrCl₂, 47% yield, unoptimized conditions) followed by cycloetherification (DBU) afforded sulfone methyl ester 35 (via transient intermediate 34), which underwent smoothly the expected intramolecular displacement at the mesylate position under the basic conditions employed (81% yield) as shown in Scheme 4. Unfortunately, however, attempts to induce the macrocyclization of p-tolylsulfone methyl ester 35 under various basic conditions (e.g., LDA,¹⁸ NaHMDS,¹⁹ KHMDS,²⁰ KOt-Bu²¹) failed to form the desired eribulin precursor 36, leading to the search for an alternative approach.

### III. Second and Successful Attempt toward Eribulin (3) from Building Block 28 and Fragment IJKLMN

Having failed to complete the synthesis of eribulin (3) from building block 28 via fragment A′ (33, Scheme 4), a new approach to reach our target molecule was adopted, eribulin (3), by employing a new version of fragment A (i.e., A″, 38) and fragment UJKLMN (8) as shown in Scheme 5. The new strategy envisioned an iodoaldehyde substrate 44 (Scheme 5) as a precursor to eribulin's macrocyclic structural motif through an intramolecular C—C bond formation. To this end, the desired fragment A″ (38, Scheme 5) was synthesized from readily available building block 28 (for its synthesis, see Scheme 3) as summarized in Scheme 5. Thus, 28 was first converted to fully protected acetonide tetrahydrofuran 37 through a four-step sequence involving, a) selective PMB protection of its primary alcohol (PMB-TCAI); b) methylation of its secondary alcohol (NaH, MeI); c) diastereoselective dihydroxylation (AD-mix-α, MeSO₂NH₂); and d) acetonide protection [p-TsOH H₂O, 2,2-dimethoxypropane (2,2-DMP)] of the so-generated diol, in 45% overall yield for the four steps as depicted in Scheme 5. Desilylation of 37 (TBAF) followed by Dess-Martin oxidation (DMP) of the resulting primary alcohol furnished coveted aldehyde fragment A″ (38) in 88% overall yield [13 steps from 23 as opposed to 16 steps for fragment A′ (33) from the same building block 23, see Scheme 3]. Coupling of fragment A″ (38) with fragment IJKMAN (8) by Nozaki-Hiyama-Kishi reaction^{6,7,22} [NiCl₂/CrCl₂ ligand (−)-39, optimized conditions] followed by cycloetherification (DBU) afforded PMB protected ester 40 in 56% overall yield. Oxidative cleavage (DDQ, 71% yield) of the PMB protective group within 40 furnished precursor 41 for further advancement to the targeted eribulin molecule (3, Scheme 5).

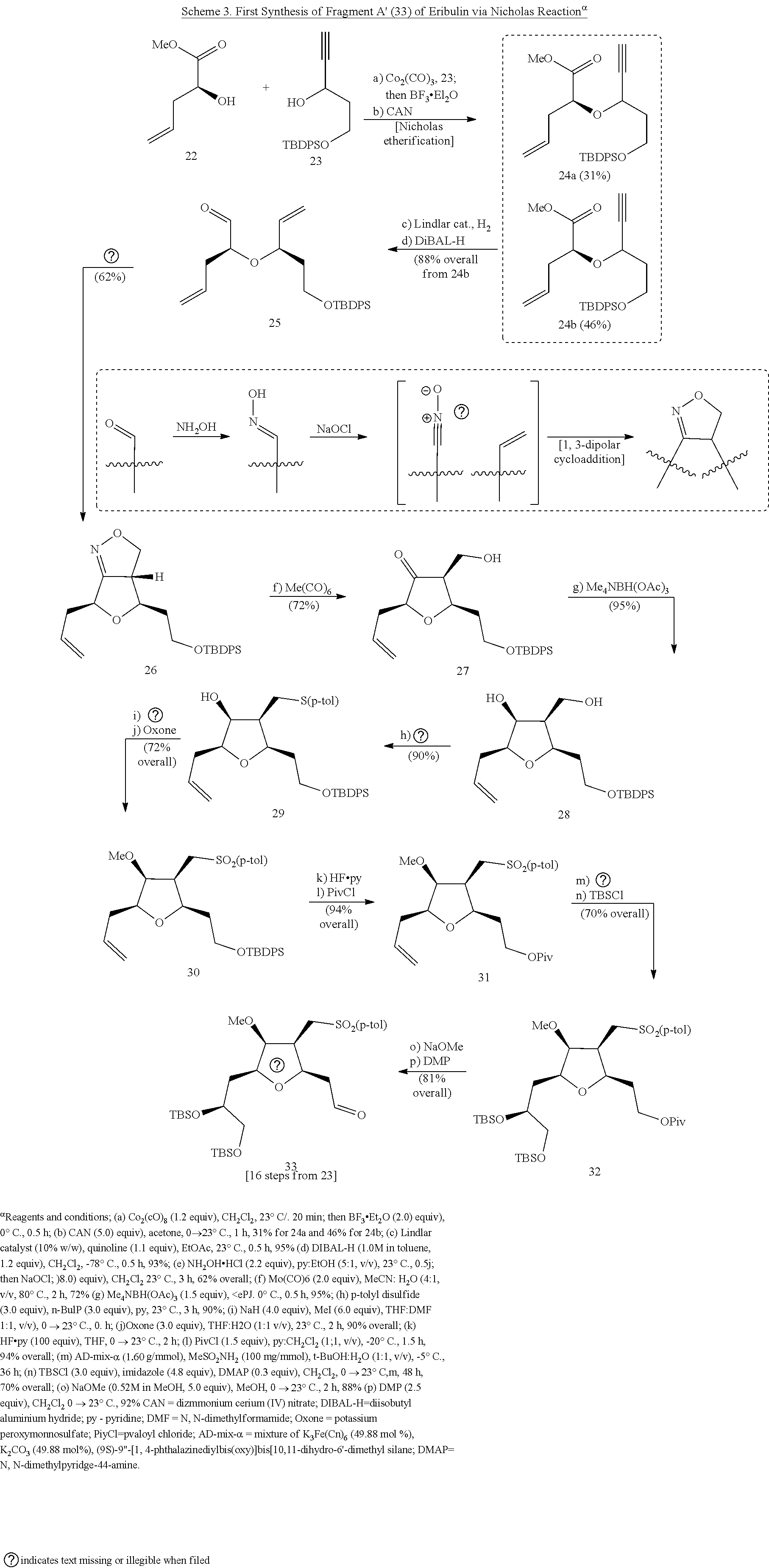

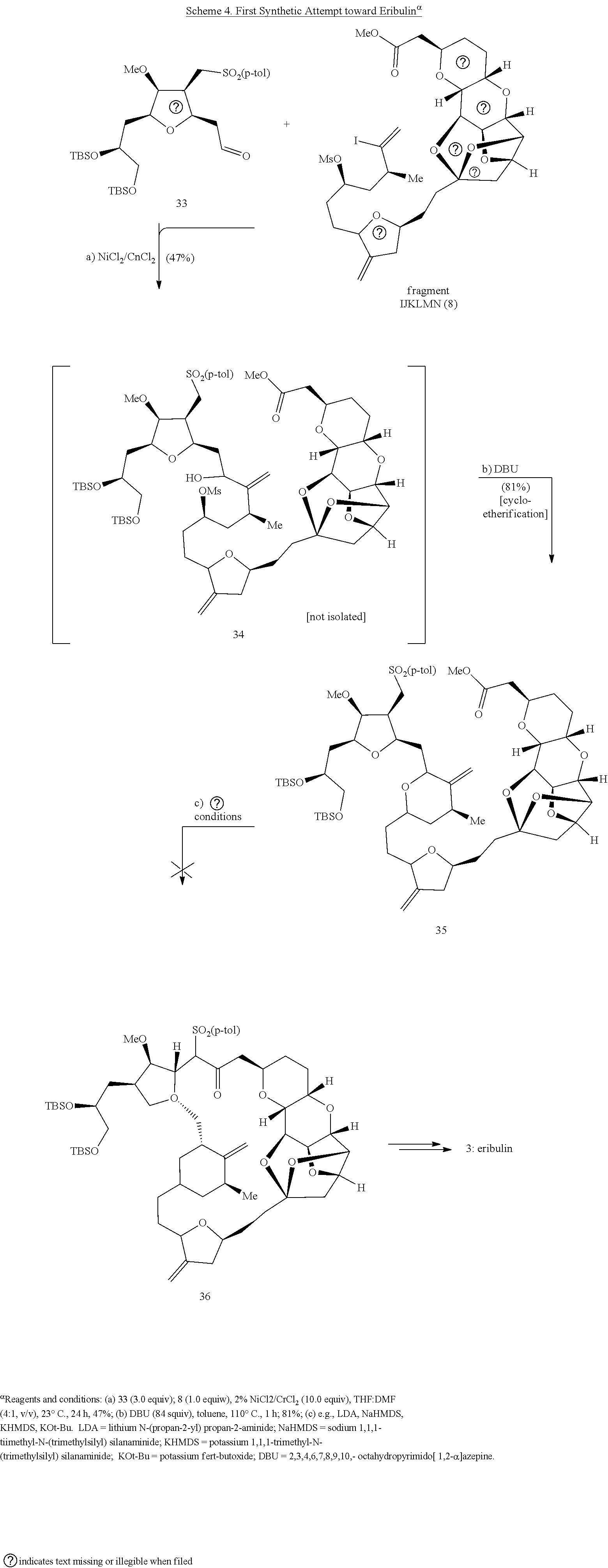

Forming the eribulin macrocycle through the remaining C—C bond was a challenge, given the limited number of methods available for constructing such an all-carbon ring.²³ In the halichondrin and eribulin field, Kishi and Eisai scientists employed the intramolecular Nozaki-Hiyama-Kishi reaction (vinyl iodide-aldehyde coupling), the ring-closing metathesis (RCM), and the Horner-Wadsworth-Emmons olefination to form the related macrocycle within their target molecules.²⁴ In this case, and in order to enrich the toolbox for constructing such challenging all carbon ring macrocycles, there was a need to search for a new method to achieve such macrocyclizations. To this end, key advanced intermediate 41 were converted to various potential precursors (i.e., 42, 44, and 45) for casting the required C—C bond of the targeted macrocycle, as shown in Scheme 5. Thus, Dess-Martin oxidation of the primary hydroxyl group within 41 led to aldehyde methyl ester 42 in 90% yield, whereas iodination (I₂, PPh₃) of 41 at the hydroxyl site, followed by DIBAL-H induced selective reduction of the so-formed iodomethyl ester, furnished iodoaldehyde 44 in 65% overall yield, as shown in Scheme 5.

TABLE 1
Screening and Optimization of the Macrocyclization Reaction of 44ª

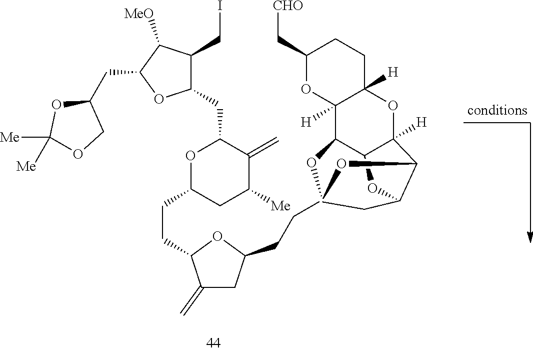 [SMILES: C=C1C[C@H](CC[C@H]2O[C@@H]3[C@@H]4O[C@H](CC)[C@H](O2)[C@@H]4O[C@H]2CC[C@H](CC=O)O[C@H]32)O[C@H]1CC[C@H]1C[C@@H](C)C(=C)[C@@H](C[C@@H]2O[C@H](C[C@H]3COC(C)(C)O3)[C@H](OC)[C@H]2CI)O1]

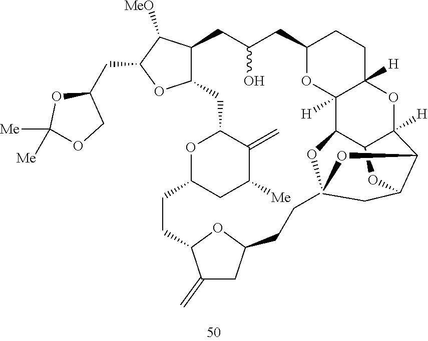 [SMILES: C=C1C[C@@H]2CC[C@H]3O[C@@H]4[C@@H]5O[C@H](CC)[C@H](O3)[C@@H]5O[C@H]3CC[C@H](CC(O)C[C@@H]5[C@@H](OC)[C@@H](C[C@H]6COC(C)(C)O6)O[C@H]5C[C@H]5O[C@@H](CC[C@@H]1O2)C[C@@H](C)C5=C)O[C@H]43]

| entry | reagent(s) | additive | solvent | ϑ/° C. | yield (47) |
|---|---|---|---|---|---|
| 1 | SmI₂ | — | THF | −78 → 23 | n.d. [symbol] |
| 2 | Sml₂ | HMPA | THF | −78 → 23 | <15%^{b} |
| 3 | t-BuLi | — | THF | −78 | n.d.^{c} |
| 4 | AIBN/n-Bu₃SnH | — | toluene | 90 | n.d.^{d} |
| 5 | NiCl₂/CrCl₂ | — | DMF or DMSO | 23 | n.d. |
| 6 | NiCl₂/CrCl₂ | (~)-39 | THF | 23 | n.d. |
| 7 | CoPc/CrCl₂ | (~)-39 | MeCN or DME: | 23 | <20% |
| 8 | CoPc/CrClz | — | DMF | 23 | 41% |
| 9 | VB₁₂/CrCl₂ | — | DMF | 23 | 42% [symbol] |
| 10 | CoPc/CrCl₂ | — | DMF | 40 | 37% |
| 11 | CoPc/CrCl₂ | — | DMF | 80 | 43% |
| 12 | CoPc/CrCl₂ | — | DMF | 23 | 33%^{f} |
| 13 | CoPc/CrCl₂ | — | DMF | 23 | 43%^{g} |
| 14 | CoPc/CrCl₂ | — | DMF | 23 | 47%^{h} |
| 15 | CoPc/CrCl₂ | 4 Å MS | DMF | 23 | 27% |
| 16 | CoPc/CrCl₂ | KI | DMF | 23 | 53% |
| 17 | CoPc/CrCl₂ | KI | DMF | 23 | 67%ⁱ |

^{a}Conditions: all reactions were carried out with 44 (1.0 mg, 1.1 μmol, 1.0 equiv), solvent (2.0 mL), 23-80° C., 48 h; for entries 5-17, NiCl₂ (2.0 equiv) or CoPc (1.0 equiv), CrCl (50 equiv), additive (20 equiv);
^{b}SmI₂ (90 equiv);
^{c}t-BuLi (2.0-5.0 equiv);
^{d}AIBN (1.0 equiv), n-Bu₃SnH (5.0 equiv);
^{e}VB₁₂ (0.1 equiv);
^{f}CoPc (0.2 equiv);
^{g}CoPc (3.0 equiv);
^{h}CrCl₂ (150 equiv);
ⁱCrCl₂ (100 equiv).
n.d. = not detected;
HMPA = hexamethylphosphoric triamide;
AIBN = 2,2′-(1,2-diazenediyl)bis[2-methylpropanenitrile];
DMSO = (methanesulfinyl)methane;
DME = 1,2-dimethoxyethane;
CoPc = (SP-4-1)-[29H,31H-phthalocyaninato(2-)-κN²⁹, κN³⁰, κN³¹, κN³²]cobalt, VB₁₂ = vitamin B₁₂;
MS = molecular sieves. [symbol]
indicates data missing or illegible when filed

Attempts to induce macrocyclization of aldehyde methyl ester 42 employing SmI₂²⁵ (or SmI₂/HMPA, or SmI₂/LiCl) unfortunately failed to produce the expected macrocycle 43 as depicted in Scheme 5. Iodoaldehyde 44 was then converted to its TBS-protected and KCN (87% yield), as a potential precursor of the expected macrocyclic TBS-protected hydroxynitrile derivative 46 as shown in Scheme 5. However, several attempts to facilitate the intramolecular C—C bond formation required to form macrocycle 46 (Scheme 5) inder basic conditions (e.g., LDA, LiHMDS, DBU, etc.) proved fruitless, leading us to attempt the SmI₂/HMPA and other protocols including CoPc/CrCl₂ conditions with alkyl iodoaldehyde 44, as the precursor (Table 1, entries 1-9). The early screening studies revealed the Takai conditions²⁶ (CoPc/CrCl₂ and VB₁₂/CrCl₂, Table 1, entries 8 and 9, highlighted beige) to be the most promising for further optimization, and with the CoPc/CrCl₂ chosen as the simplest and more convenient combination of reagents for our purpose. As shown in Table 1, the use of CoPc/CrCl₂ combination of reagents produced the desired macrocyclic product 47 in moderate to good yields (Table 1, entries 10-16, highlighted blue), with 67% yield being the best yield obtained, by employing KI as an additive and adjusting the equivalents of CrCl₂ (Table 1, entry 17, highlighted green). Whereas previous work in the halichondrin, eribulin and other carbocyclic systems employed the Nozaki-Hivama-Kishi reaction²⁷ (e.g., utilizing a vinyl halide-aldehyde and alkynyl halide-aldehyde coupling, respectively) to cast the relevant macrocycles, the present macrocyclization features an intramolecular coupling between an alkyl halide and an aldehyde functionality, thus representing a new paradigm for the construction of such challenging structural motifs, although the intermolecular version of this reaction was previously demonstrated by Takai et al.²⁶

With the macrocycle construction problem solved, eribulin (3) was in reach with only a three-step sequence remaining to complete its total synthesis. Thus, as shown in Scheme 5, hydroxy-acetonide 47 was treated with Dess-Martin reagent to achieve the pending oxidation of the hydroxyl group to its ketone counterpart followed by cleavage of the acetonide moiety from the latter precursor (AcOH) to furnish diol 48 in 81% overall yield for the two steps. Finally, diol 48 was converted to eribulin (3) through selective tosylation (Ts₂O), followed by amnination's (NH₄OH), in 68% overall yield as depicted in Scheme 5.

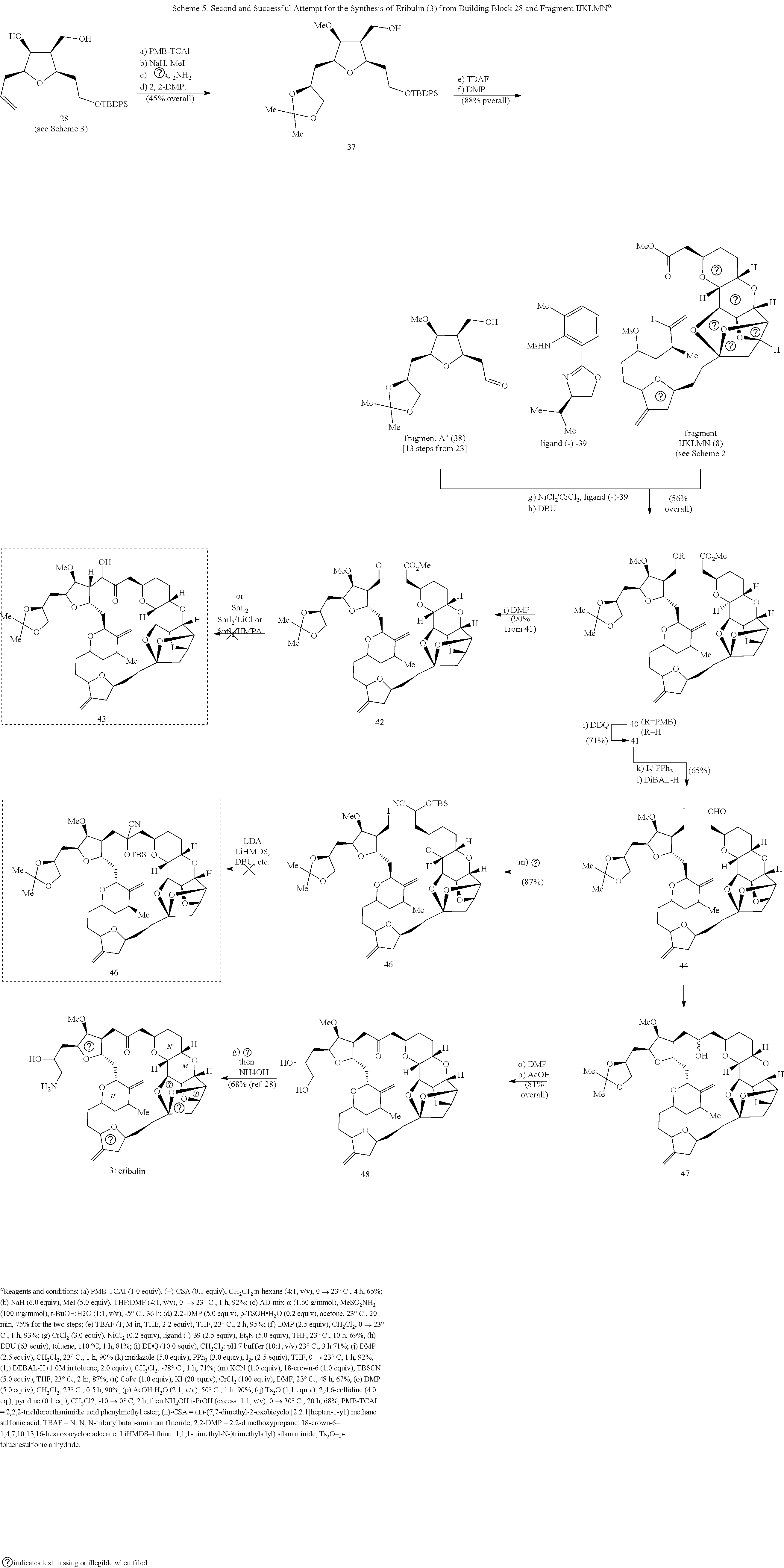

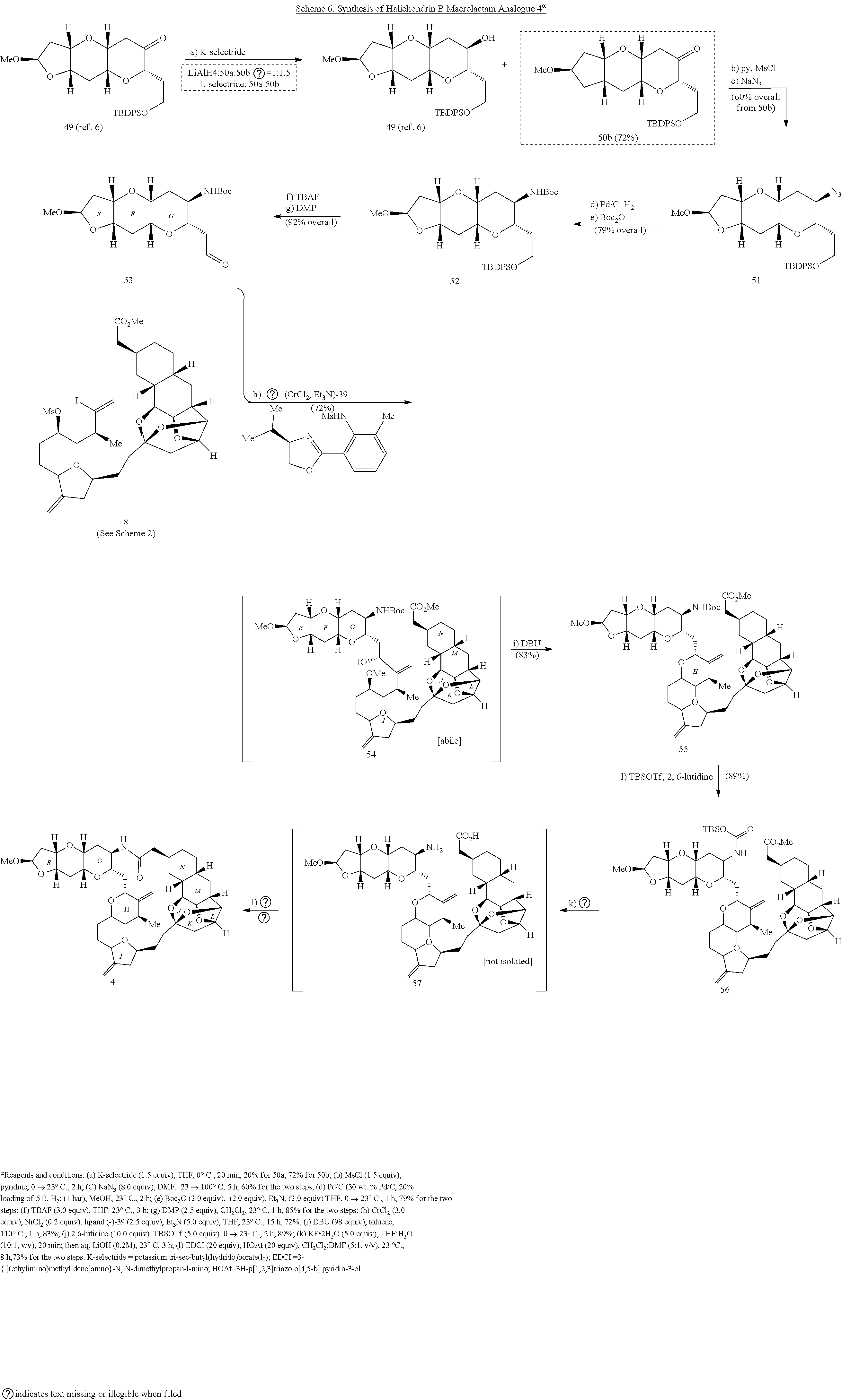

### IV. Synthesis of Halichondrin B Macrolactam Analogue 4

In an effort to further expand the scope of the synthetic strategy beyond eribulin and into the realm of novel analogues of the halichondrins, the synthesis of macrolactam 4 was undertaken and accomplished as summarized in Scheme 6. Thus, stereoselective reduction of tricyclic ketone 49⁶ with K-selectride led to diastereomeric and chromatographically separable alcohols 50a (20% yield) and 50b (72% yield). Mesylation (MsCl, py) of the major and desired isomer 50b followed by azide formation (NaN₃), accompanied b_{y} stereochemical inversion, led to azide 51 in 60% overall yield. Reduction of the latter (Pd/C, H₂) followed by Boc-protection of the resulting amine (Boc₂O, Et₃N) furnished TBDPS,Boc-bis-protected derivative 52 (79% overall yield), whose desilylation (TBAF) and oxidation of the resulting primary alcohol (DMP) led to aldehyde 53 (fragment EFG) in 92% overall yield for the two steps as depicted in Scheme 6. Coupling of fragments EFG (53, equipped with a protected primary amine) and LJKJMAN fragment (8, see Scheme 2 for preparation) through Nozaki-Hiyana-Kishi reaction [NiCl₂/CrCl₂, Et₃N, ligand (−)-39, 72% yield] furnished coupling product 54. The latter transient, intermediate was subjected to cycloetherification to cast ring H in the growing molecule (DBU, 83% field), affording advanced intermediate 55. The latter was then transformed to its silyl carbamate counterpart (56) by exposure to TBSOTf and 2,6-lutidine,²⁹ in 89% yield, as shown in Scheme 6. Finally, precursor 56 was transformed to the coveted macrolactam halichondrin analogue 4, in 73% overall yield, through transient amino acid 57, first by exposure to KF·2H₂O and LiOH (liberation of the amino and carboxyl group, respectively) and thence to EDCI and HOAt (macrolactam formation).

### V. Analogs

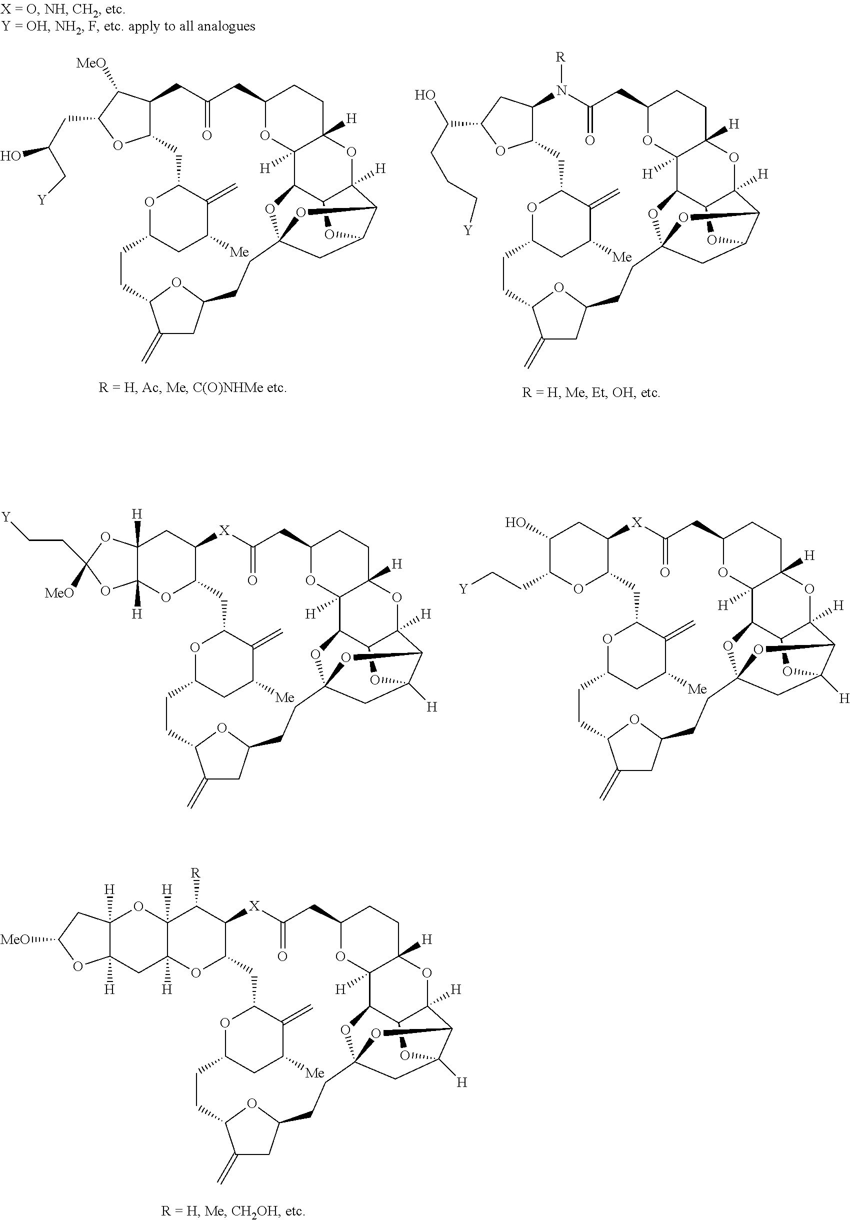

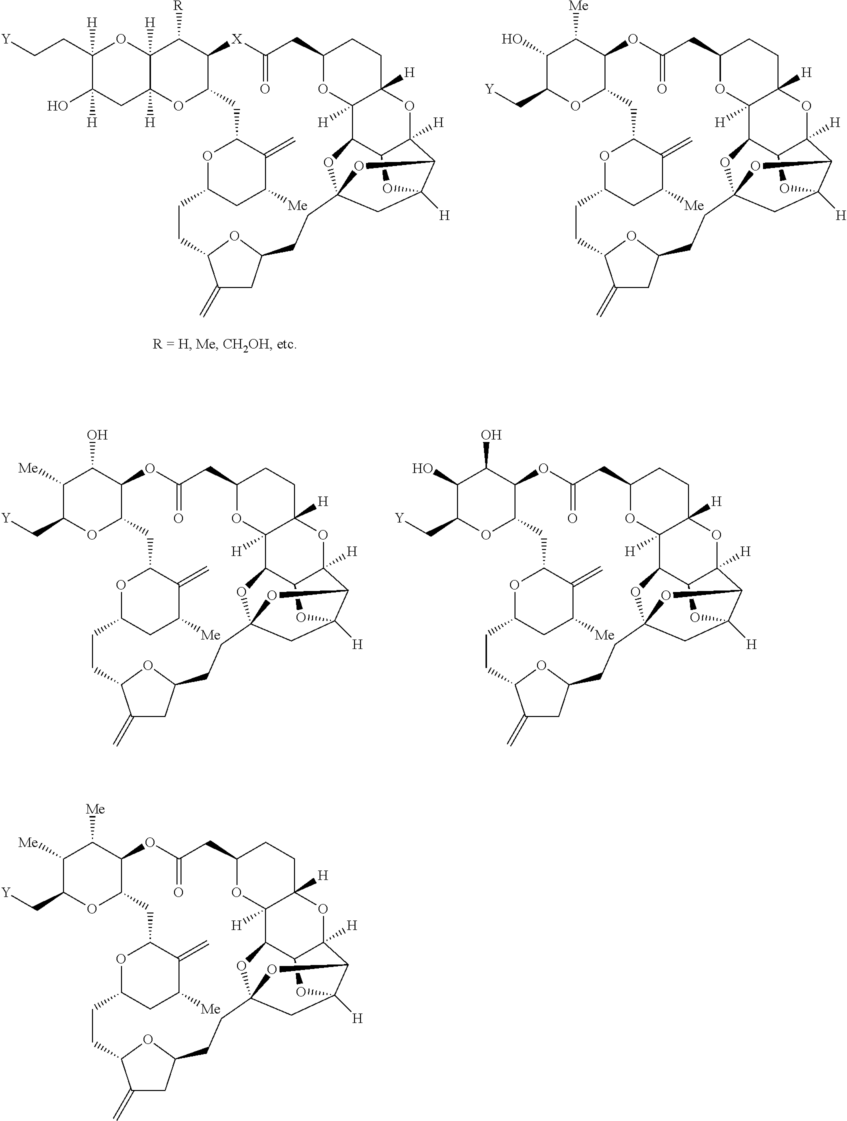

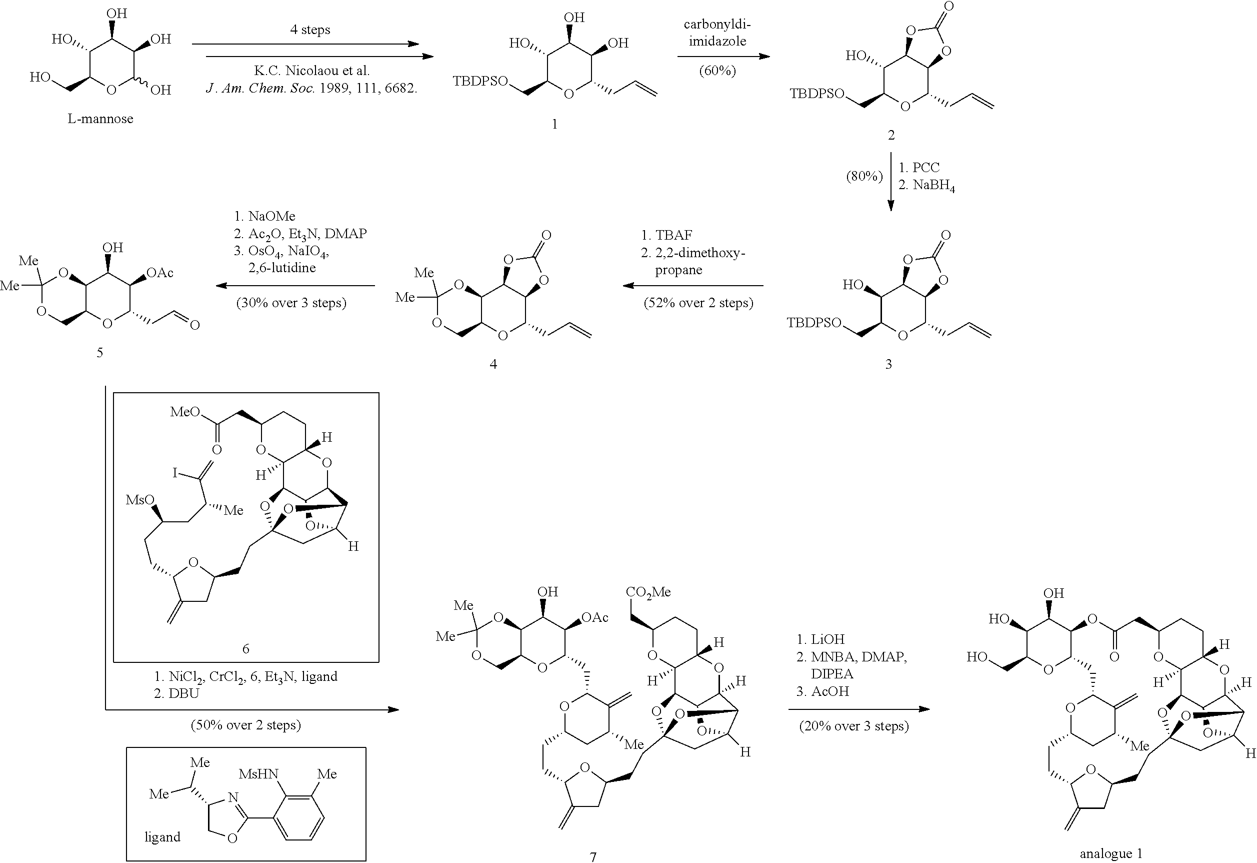

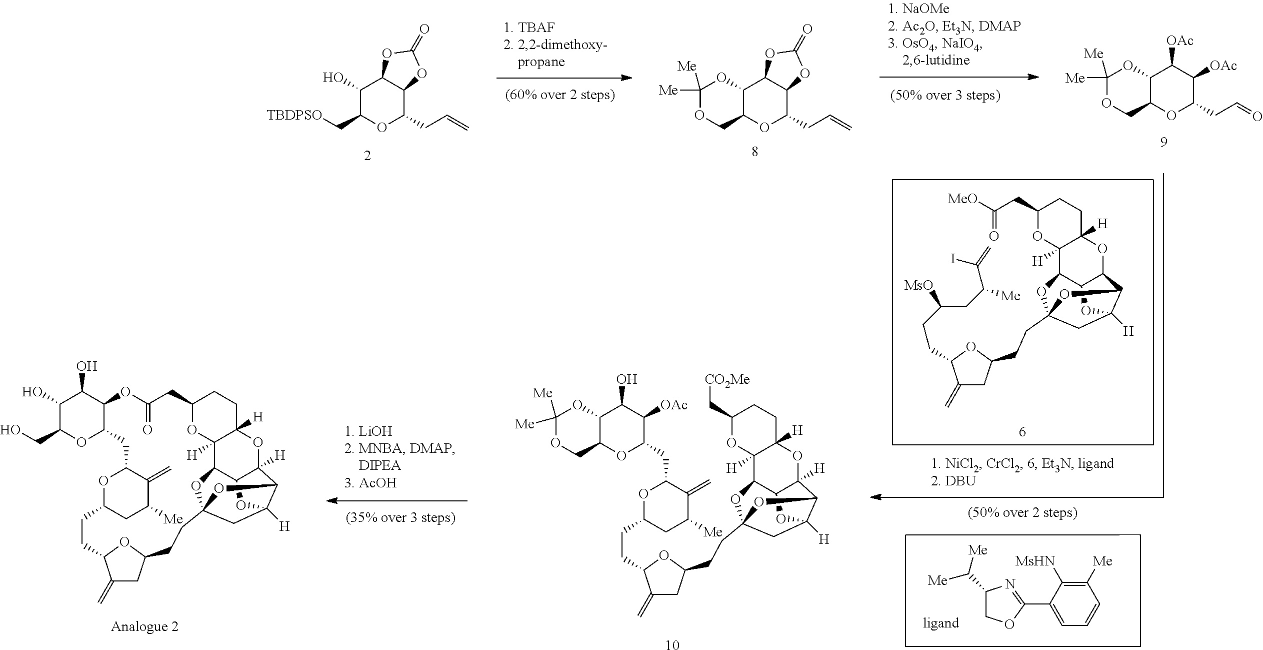

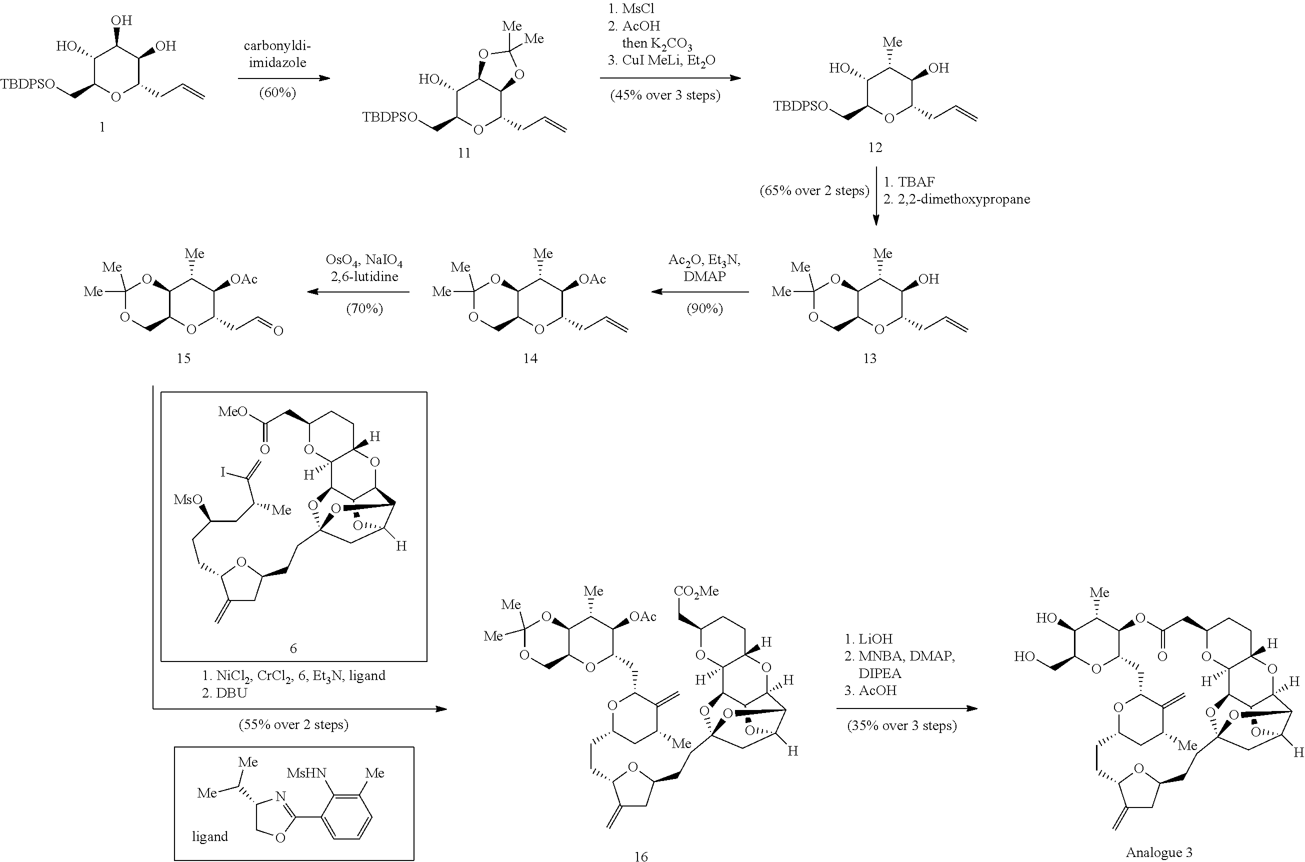

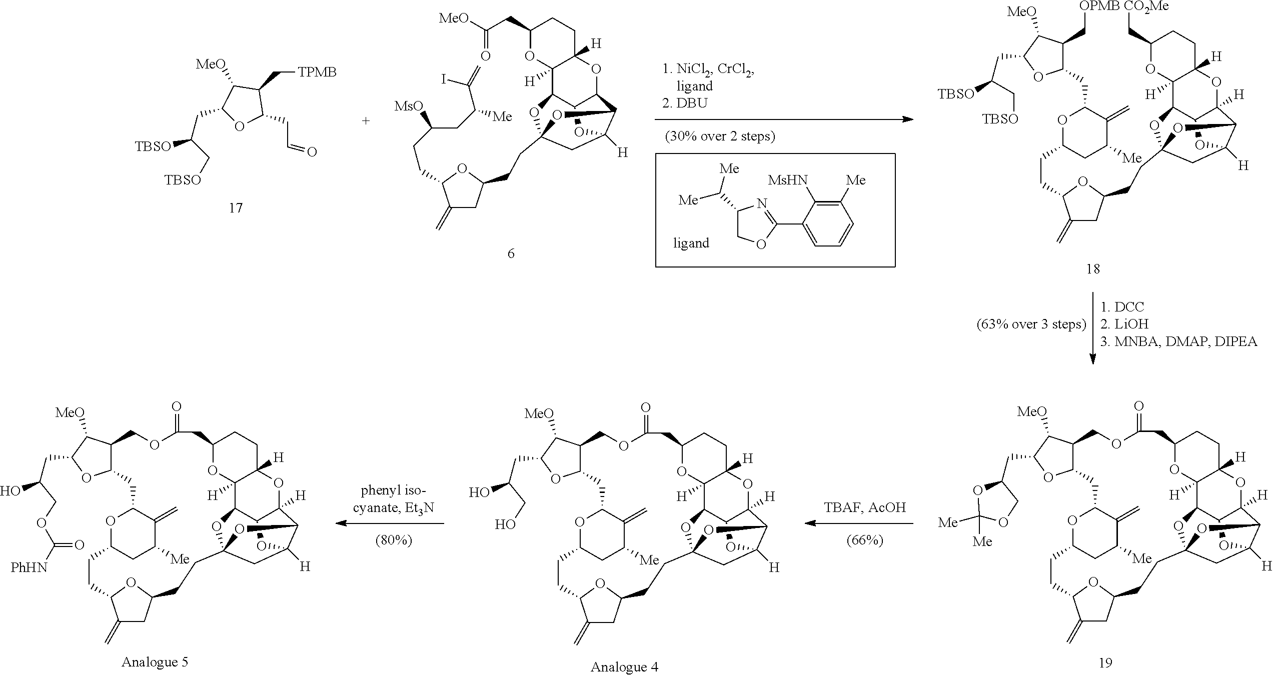

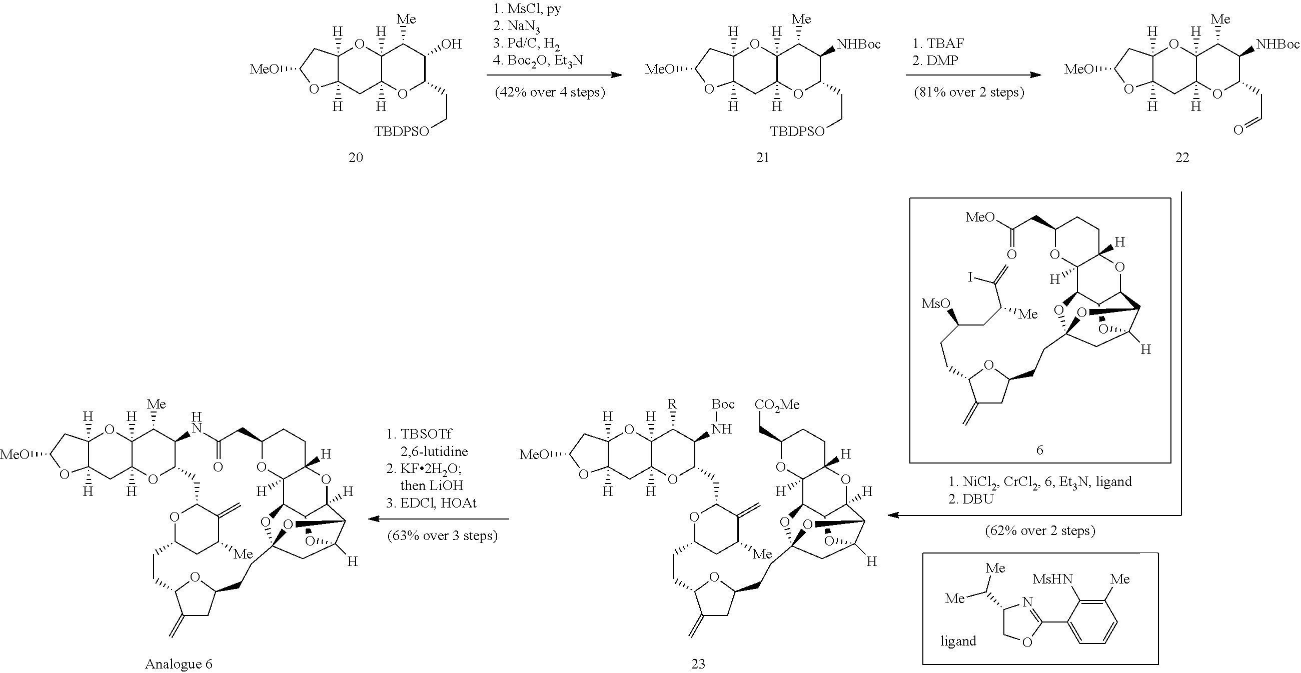

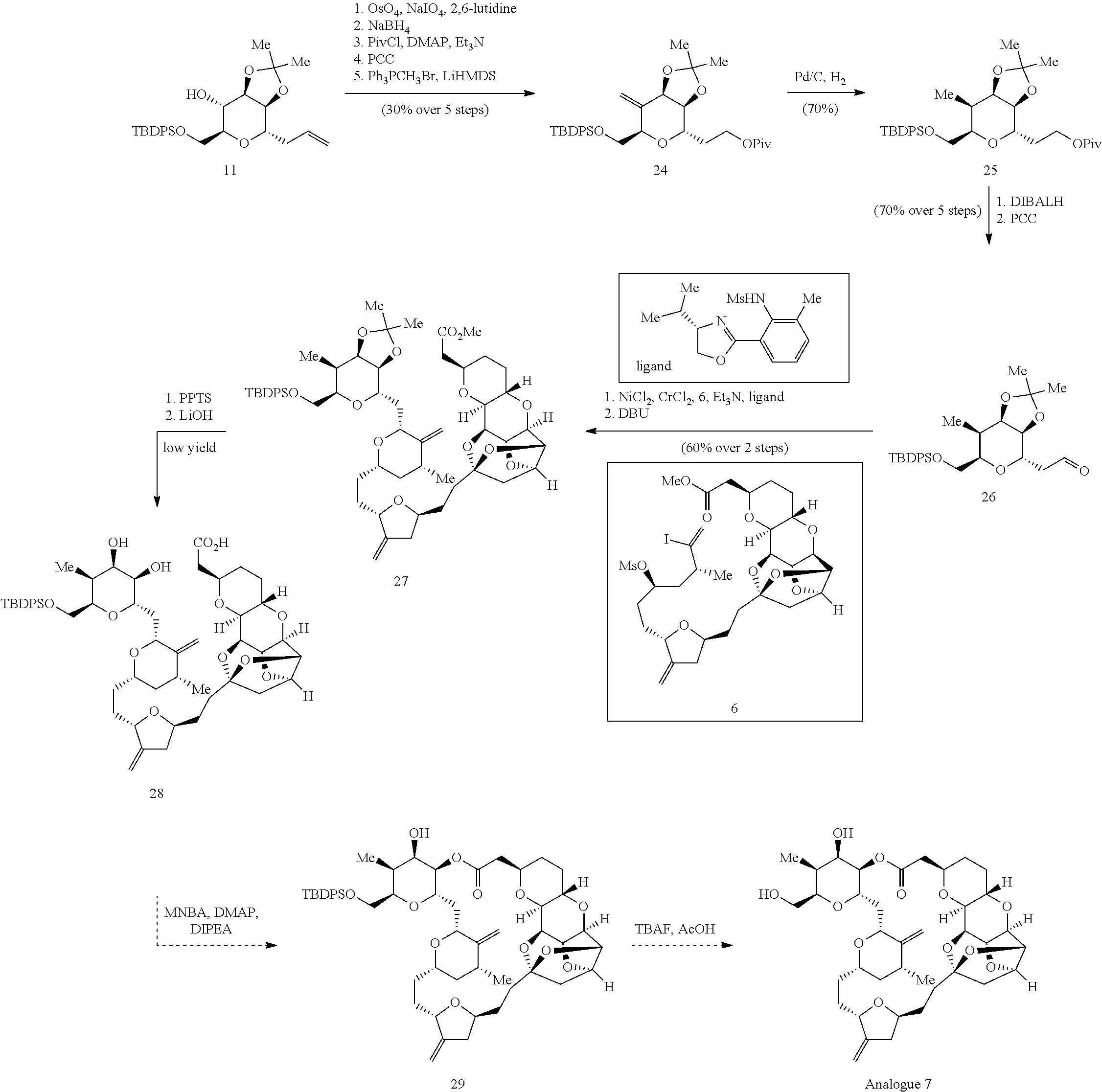

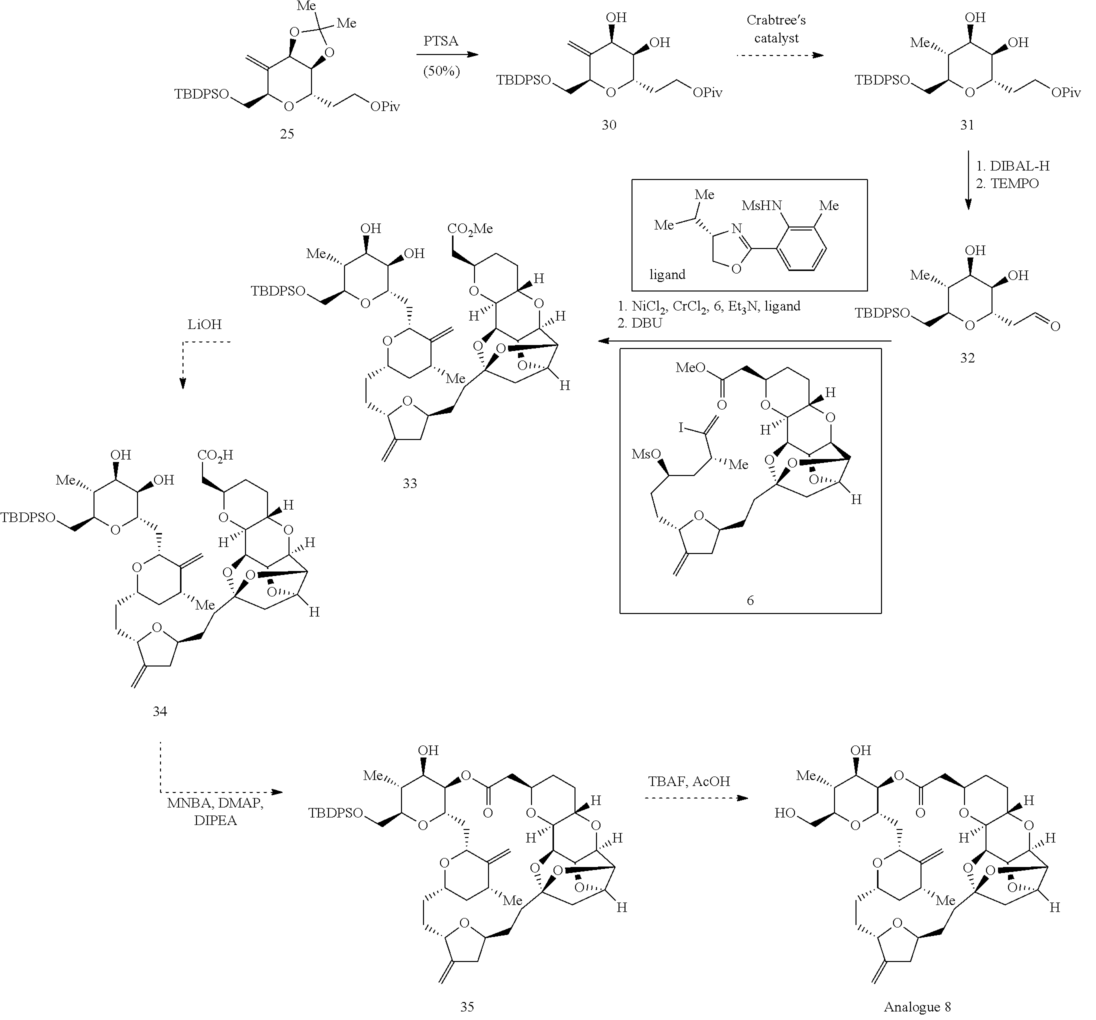

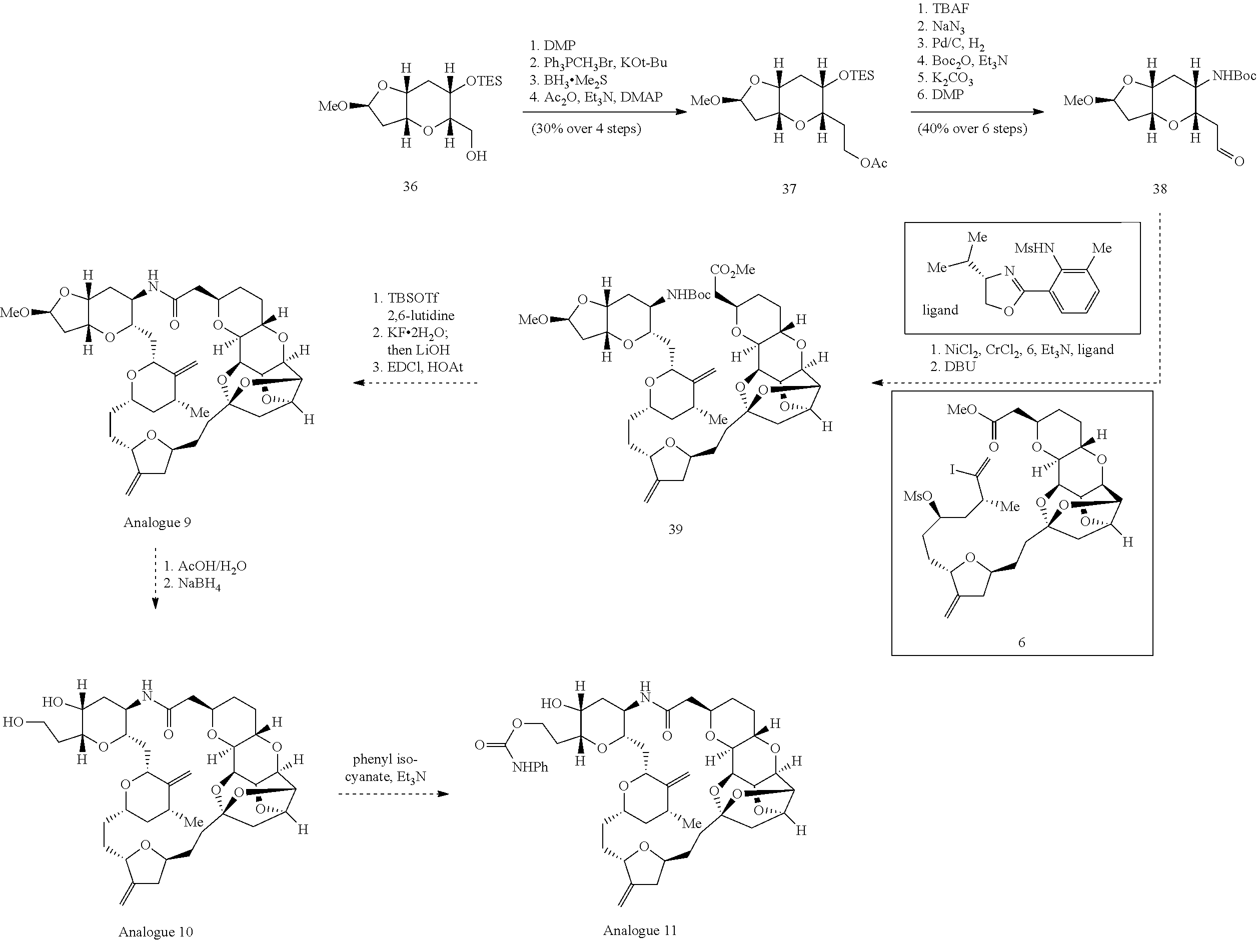

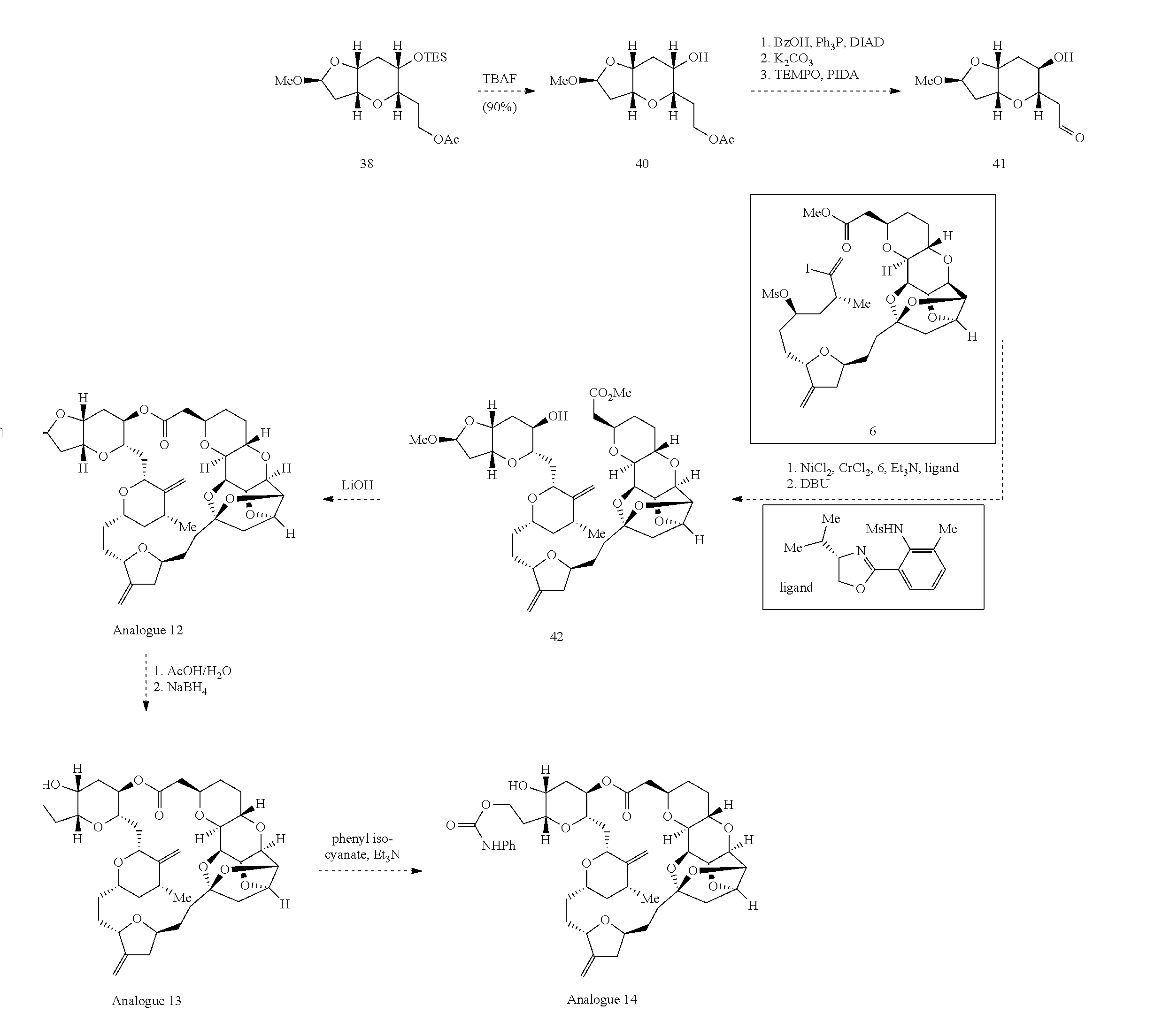

### VI. General Information

All reactions were carried out under an argon atmosphere with dry solvent under anhydrous conditions, unless otherwise noted. Dry acetonitrile (MeCN), dimethylformamide (DMF), dimethyl sulfoxide (DMSO), dichloromethane (CH₂Cl₂) tetrahydrofiran (THF) and toluene were obtained by passing commercially available pre-dried, oxygen-free formulations through activated alumina columns. Anhydrous benzene, acetone, chloroform (CHCl₃), methanol (MeOfH), ethyl acetate (EtOAc) were purchased from commercial suppliers and stored under argon. Yields refer to chromatographically and spectroscopically (¹H NMR) homogenous material, unless otherwise stated. Reagents were purchased at the highest commercial quality and used without further purification, unless otherwise noted. Reactions were monitored by thin-layer chromatography (TLC) carried out on S-2 0.25 mm E. Merck silica gel plates (60 F₂₅₄) using UV light for visualization and/or an ethanolic solution of phosphomolybdic acid, an aqueous solution of cerium sulfate or a basic aqueous solution of potassium permanganate as developing agents. E. Merck silica gel (60, particle size 0.040-0.063 mm) was used for flash column chromatography. NMR spectra were recorded on a Bruker DRX-600 instrument and calibrated using the resonance signal of the residual undeuterated solvent for ¹H NMR [δ_{H}=7.26 ppm (CDCl₃), S_{H}=7.16 ppm (C₆D₆), and δ_{H}=3.31 ppm (CD₃OD)] and deuterated solvent for ¹³C NMR [δ_{C}=77.16 ppm (CDCl₃), δ_{C}=128.06 ppm (C₆D₆), and δ_{C}=49.00 ppm (CD₃OD)] as an internal reference at 298K. The following abbreviations were used to designate multiplicities: s=singlet, d=doublet, t=triplet, q=quartet, quint=quintet, m=multiplet, br=broad. Infrared (1R) spectra were recorded on a PerkinElmer 100 FT-IR spectrometer. High-resolution mass spectra (HRMS) were recorded on an Agilent ESI-TOF (time of flight) mass spectrometer using MALDI (matrix-assisted laser desorption ionization) or ESI (electrospray ionization). Optical rotations were measured on a Schmidt+Haensch Polartronic M100 polarimeter at 589.44 nm using 100 mm cells and the solvent and concentration indicated, and are reported in units of 10⁻¹ (deg cm² g⁻¹).

### VII. Experimental Procedures and Characterization Data

As shown in Scheme S1, the synthesis of fragment MN evolved from our initial trial (route a), which was elaborated into the previous reported process (route b), (Nicolaou et ad., 2021) and finally improved to the one reported in the current work (route c).

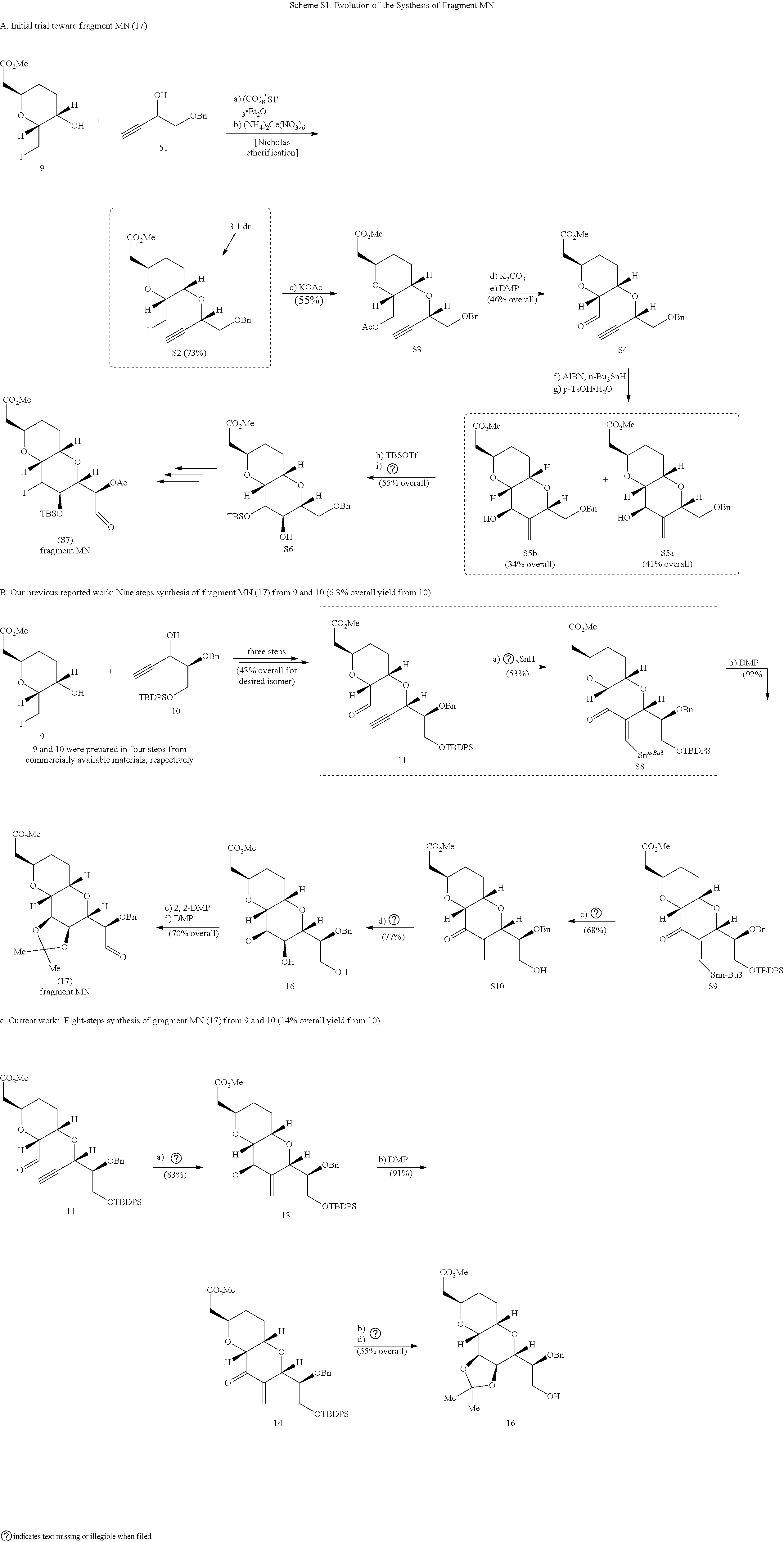

Methyl 2[(R,5S,6S)-5-{[(2R)-1-(benzyloxy)but-3-yn-2-yl]oxy}-6-(iodomethyl)tetrahydro-2H-pyran-2-yl]acetate (S2): To a stirred solution of alcohol S1 (2.96 g, 16.8 mmol, 1.0 equiv) in CH₂Cl₂ (20 mL) at 23° C. was added Co₂(CO)₈ (6.87 g, 20.1 mmol, 1.2 equiv). The resulting mixture was stirred

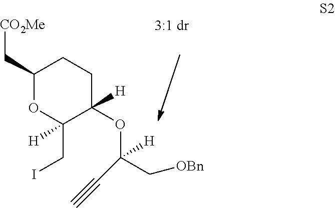 [SMILES: C#C[C@H](COCc1ccccc1)O[C@H]1CC[C@H](CC(=O)OC)O[C@@H]1CI]

for 1 h before it was cooled to 0° C. Then, hydroxy ester 9 (15.8 g, 50.4 mmol, 3.0 equiv) was added and the resulting mixture was stirred for 10 mm, before BF₃·Et₂O (5.20 mL, 42.0 mmol, 2.5 equiv) was added. The reaction mixture was stirred for additional 3 h before it was quenched by addition of NaHCO₃ solution (200 mL, sat. aq.). The layers were separated, and the aqueous layer was extracted with CH₂Cl₂ (3×200 mL). The combined organic extracts were washed with brine (100 mL), dried over Na₂SO₄ and concentrated under reduced pressure. The obtained residue (cobalt complex of the product) was used directly in the next step.

To a stirred solution of the above obtained cobalt complex in acetone (150 mL) at 0° C. was added ceric ammonium nitrate (46.0 g, 82.0 mmol, 5.0 equiv) in three potions in 10-minute intervals and the resulting mixture was warned to 23° C. and stirred for 1 h. The resulting mixture was concentrated under reduced pressure. The obtained residue was then diluted with H₂O (100 mL) and extracted with EtOAc (3×150 mL). The combined organic extracts were subsequently washed with H₂O (100 mL), brine (60 mL), dried over Na₂SO₄, and concentrated under reduced pressure. Flash column chromatography (SiO₂, hexanes/EtOAc 10:1, v/v→1:1, v/v) of the residue afforded iodo ester S2 (mixture of isomers, 3:1 dr, 5.81 g. 12.0 mmol, 73% yield) and recovered 9 (11.1 g, 35.3 mmol, 70%) as colorless oils. S2: R_{f}=0.60 (SiO₂, hexanes/EtOAc 3:2, v/v);

$${[ α ]}_{D}^{23} \text{=+} 24.4 ( c = 0.9 , {CHCl}_{3} ) ; FT - IR ( film ) :$$

vₘₐₓ 3284, 2949, 2865, 1737, 1454, 1437, 1290, 1176, 1098, 1016, 739, 698 cm⁻¹; ¹H NMR (600 MHz, CDCl₃, mixture of isomers): δ 7.40-7.28 (m, 5H), 4.65-4.53 (m, 2H), 4.42-4.34 (m, 1H), 3.87-3.83 (m, 1H), 3.72 (s, 0.7H), 3.71 (s, 2.2H), 3.65-3.58 (m, 2H), 3.54 (dd, J=10.5, 2.6 Hz, 1H), 3.44 (ddd, J=102, 8.5, 4.6 Hz, 0.28H), 3.35-3.28 (m, 13H), 3.26-3.18 (m, 0.53H), 3.07 (ddd, J=9.6.7, 2.5 Hz, 0.67H), 2.58 (ddd, J=15.2, 9.1, 7.81 Hz, 1H), 2.46 (t, J=2.3 Hz, 1H), 2.45-2.40 (m, 1H), 2.36 (dq, J=12.8, 3.61 Hz, 0.661H), 2.24-2.18 (m, 0.27H), 1.84-1.77 (m, 1H), 1.68-1.57 (m, 1H), 1.49-1.37 (m, 1H) ppm; ¹³C NMR (151 MHz, CDCl₃, mixture of isomers): δ 171.62, 171.61, 137.9, 137.78, 128.59, 128.55, 127.91, 127.89, 127.83, 127.80, 81.20, 80.19, 79.7, 79.5, 78.6, 75.33, 75.28, 74.6, 74.4, 74.3, 73.62, 73.57, 72.8, 72,5, 69.5, 66.8, 52.02, 51,98, 40.72, 40.68, 30.7, 30.5, 30.1, 28.2, 8.6, 8.1 ppm; HRMS (ESI-TOF) m/z: [M+Na]⁺ Calcd. for C₂₀H₂₅IO₅Na⁺ 495.0639; Found 495.0646.
Methyl 8-O-acetyl-3,7-anhydro-6-O-[(2R)-1-(benzyloxy)but-3-yn-2-yl]-2,4,5-trideoxy-D-ribo-octonate (S3): To a stirred solution of iodo ester S2 (1.00 g, 2.12 mmol, 1.0 equiv) in DMF (10 mL) at

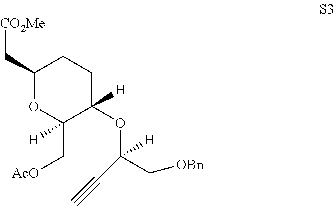 [SMILES: C#C[C@H](COCc1ccccc1)O[C@H]1CC[C@H](CC(=O)OC)O[C@@H]1COC(C)=O]

23° C. was added KOAc (825 mg, 8.48 mmol, 4.0 equiv). The reaction mixture was heated to 120° C. for 2 h before it was allowed to cool to 23° C., the resulting mixture was then diluted with H₂O (20 mL) and extracted with EtOAc (3×50 mL). The combined organic extracts were washed with brine (50 mL), dried over Na₂SO₄, and concentrated under reduced pressure. Flash column chromatography (SiO₂, hexanes/EtOAc 4:1, v/v→1:1, v/v) of the residue afforded acetate S3 (mixture of isomers, 3:1 dr, 473 mg, 1.17 mmol, 55% yield) as a colorless oil. S3: R_{f}=0.45 (SiO₂, hexanes/EtOAc 3:2, v/v);

$${[ α ]}_{D}^{23} \text{=+} 20.4 ( c = 2.3 , {CHCl}_{3} ) ; FT - IR ( film ) :$$

vₘₐₓ 2950, 2866, 1735, 1496, 1454, 1438, 1368, 1335, 1239, 1200, 1165, 1096, 1077, 1040, 1002, 912, 852, 821, 739, 699 cm⁻¹; ¹H NMR (6001 MHz, CDCl₃, desired isomer) δ 7.37-7.26 (m, 5H), 4.61-4.53 (m, 2H), 4.33 (ddd, J=11.9, 7.9, 1.8 Hz, 1H), 4.27-4.23 (m, 1H), 4.23-4.18 (m, 1H), 3.79 (dddd, J=11.2, 6.9, 5.8, 2.9 Hz, 1H), 3.68 (s, 3H), 3.63-3.55 (m, 2H), 3.48-3.44 (m, 1H), 2.59 (dd, J=15.4, 7.1 Hz, 1H), 2.45 (d, J=2.1 Hz, 1H), 2.43-2.37 (m, 2H), 2.04 (s, 3H), 1.87-1.78 (m, 1H), 1.66-1.54 (m, 2H), 1.46-1.35 (m, 1H)ppm; ¹³C NMR (151 MHz, CDCl₃, desired isomer) δ 171.6, 171.0, 137.8, 128.5, 127.84, 127.78, 81.3, 78.8, 74.8, 74.5, 74.0, 73.5, 72.7, 69.3, 64.2, 51.8, 40.6, 30.5, 30.4, 21.1 ppm; HRMS (ESI-TOF) m/z: [M+Na]⁺ Calcd. for C₁₂H₂₈O₇Na⁺ 427.1727; Found 427.1728.
Methyl 3,7-anhydro-6-O-[(2R)-1-(benzyloxy)but-3-vn-2-yl]-2,4,5-trideoxy-D-ribo-octonate and methyl 3,7-anhydro-6-O-[(2S)-1-(benzyloxy)but-3-yn-2-yl]-2,4,5-trideoxy-D-ribo-octonate (S11 and C2-epi-S11): To a stirred solution of S3 (465 mg, 1.15 mmol, 1.0 equiv) in MeOH (10 mL) at 23° C. was added anhydrous K₂CO₃ (152 mg, 1.15 mmol, 1.0 equiv). The resulting mixture was stirred for 0.5h

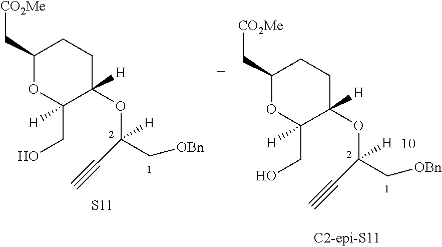 [SMILES: "S11" ; C#C[C@H](COCc1ccccc1)O[C@H]1CC[C@H](CC(=O)OC)O[C@@H]1CO ; C#C[C@H](COCc1ccccc1)O[C@H]1CC[C@H](CC(=O)OC)O[C@@H]1CO]

before it was quenched by addition of H₂O (20 mL) and extracted with EtOAc (3×50 mL). The combined organic extracts were washed with brine (30 mL), dried over Na₂SO₄, and concentrated under reduced pressure. Flash column chromatography (SiO₂, hexanes/EtOAc 4:1, v/v→3:1, v/v) of the residue afforded alcohols S11 (275 mg, 0.759 mmol, 66% yield) and C2-epi-SII (91.7 mg, 0.253 mmol, 22% yield) as colorless oils. S11: R_{f}=0.50 (SiO₂, hexanes/EtOAc 1:1, v/v);

$${[ α ]}_{D}^{23} = + 3.8 ( c = 2.8 , {CHCl}_{3} ) ;$$

FT-R (film): vₘₐₓ 3490, 3280, 2929, 2867, 2114, 1734, 1496, 1454, 1437, 1392, 1365, 1334, 1291, 1254, 1220, 1198, 1159, 1074, 1028, 989, 939, 911, 893, 847, 739, 698, 667 cm⁻¹; ¹H NMR (600 MHz, CDCl₃) δ 7.36-7.27 (m, 5H), 4.61-4.54 (m, 2H), 4.31 (ddd, J=7.9, 3.9, 2.1 Hz, 1H), 3.81 (dddd, J=11.1, 7.4, 5.5, 2.0 Hz, 1H), 3.75 (d, J=3.6 Hz, 2H), 3.67 (s, 3H), 3.62-3.54 (m, 2H), 3.50 (ddd, J=11.0, 9.3, 4.6 Hz, 1H), 3.30 (dt, J=9.3, 3.7 Hz, 1H), 2.55 (dd, J=15.4, 7.5 Hz, 2H), 2.44 (d, J=2.1 Hz, 1H), 2.43-2.35 (m, 2H), 1.80 (ddt, J=13.4, 5.0, 2.6 Hz, 1H), 1.61 (tdd, J=13.0, 11.1, 4.2 Hz, 1H), 1.43-1.34 (m, 1H) ppi; ¹³C NMR (151 MHz, CDCl₃) δ 171.6, 137.5, 128.6, 128.00, 127.97, 81.1, 80.6, 76.1, 74.4, 74.0, 73.6, 72.7, 69.4, 63.0, 51.8, 40.7, 30.7, 30.2 ppm; HRMS (ESI-TOF) m/z: [M+Na]⁺ Calcd. for C₂₀H₂₆O₆Na⁺ 385.1622; Found 385.1627. C2-epi-SII: R_{f}=0.49 (SiO₂, hexanes/EtOAc 1:1, v/v);

$${[ α ]}_{D}^{23} = + 52.3 ( c = 2.4 , {CHCl}_{3} ) ;$$

FT-IR (film): vₘₐₓ3500, 3279, 2930, 2868, 2112, 1735, 1496, 1454, 1438, 1366, 1335, 1292, 1256, 1220, 1199, 1158, 1093, 1077, 1029, 952, 911, 892, 848, 740, 699 cm⁻¹, ¹H NMR (600 MHz, CDCl₃) δ 7.37-7.27 (m, 5H), 4.65-4.56 (m, 2H), 4.40 (ddd, J=6.1, 5.1, 2.1 Hz, 1H), 3.88-3.80 (m, 2H), 3.71 (dd, J=11.7, 5.4 Hz, 1H), 3.68 (s, 3H), 3.64-3.56 (m, 3H), 3.35 (ddd, J=9.1, 5.4, 3.4 Hz, 1H), 2.57 (dd, J=15.4, 7.4 Hz, 1H), 2.47 (d, J=2.1 Hz, 1H), 2.43 (dd, J=15.4, 5.6 Hz, 1H), 2.27-2.21 (m, 1H), 2.05-1.89 (m, 1H), 1.86-1.79 (m, 1H), 1.51-1.33 (m, 2H) ppm; ¹³C NMR (151 MHz, CDCl₃) δ 171.6, 137.9, 128.6, 127.9, 127.8, 80.32, 80.26, 75.0, 73.9, 73.7, 72.5 (2 C), 66.9, 63.3, 51.9, 40.7, 30.4, 28.4 ppm; HRMS (ESI-TOF) m/z: [M+Na]⁺ Calcd. for C₂₀H₂₆O₆Na⁺ 385.1622; Found 385.1624.
Methyl 2,6-anhydro-3-O-[(2R)-1-(benzyloxy)but-3-yn-2-yl]-4,5,7-trideoxy-L-ribo-octuronate (S4): To a stirred solution of alcohol S11 (260 mg, 0.717 mmol, 1.0 equiv) in dry CH₂Cl₂ (10 mL) at

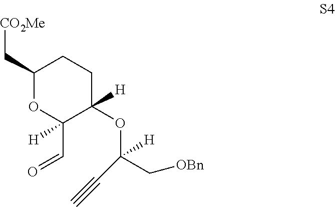 [SMILES: C#C[C@H](COCc1ccccc1)O[C@H]1CC[C@H](CC(=O)OC)O[C@@H]1C=O]

0° C. was added NaHCO₃ (363 mg, 4.30 mmol, 6.0 equiv) and Dess-Martin periodi-nane (912 mg, 2.15 mmol, 3.0 equiv). The resulting mixture was warmed to 23° C. and stirred for 4 h before it was diluted with H₂O (10 mL) and Na₂S₂O₃ solution (20 mL, sat. aq.). The resulting mixture was stirred vigorously for 3 h before the layers were separated. The aqueous layer was extracted with CH₂Cl₂ (3×50 mL) and the combined organic extracts were washed with brine (30 mL), dried over Na₂SO₄, and concentrated under reduced pressure. Flash column chromatography (SiO₂, hexanes/EtOAc 5:1, v/v→1:1, v/v) of the residue afforded aldehyde S4 (180 mg, 0.502 mmol, 70% yield) as a colorless oil. S₄: R_{f}:=0.60 (SiO₂, hexanes/EtOAc 1:1, v/v);

$${[ α ]}_{D}^{23} = - 8.5 ( c = 0.5 , EtOAc ) ;$$

FT-IR (film): vₘₐₓ 3468, 3273, 2950, 2866, 1737, 1454, 1438, 1290, 1199, 1094, 741 cm⁻¹; ¹H NMR (600 MHz, C₆D₆): δ 9.56 (d, J=1.3 Hz, 1H), 7.30-7.24 (m, 2H), 7.20-7.16 (m, 2H, one proton resonance signal overlaps with the residual ¹H resonance signal of the solvent), 7.10-7.04 (m, 1H), 4.34-4.23 (m, 3H), 3.54 (dd, J=10.3, 7.2 Hz, 2H), 3.50-3.43 (m, 3H), 3.30 (s, 3H), 2.41 (dd, J=15.6, 7.3 Hz, 1H), 2.14 (dq, J=12.4, 3.8 Hz, 1H), 2.07 (dd, J=15.6, 5.5 Hz, 1H), 1.97 (d, J=2.1 Hz, 1H), 1.43-1.32 (m, 1H), 1.32-1.24 (m, 1H), 1.01-0.91 (m, 1H)ppm; ¹³C NMR (151 MHz, CDA): δ 197.4, 170.6, 138.6, 128.6, 128.4, 127.8, 83.6, 81.8, 744, 73.9, 73.8, 73.4, 73.2, 69.5, 51.2, 40.4, 30.6, 29.8 ppm; HRMS (ESI-TOF) m/z: [M+Na]⁺ Calcd. for C₂₀H₂₄O₆Na⁺ 383.1465; Found 383.1466.
Methyl 3,7:6,10-dianhydro-11-O-benzyl-2,4,5,9-tetradeoxy-9-methylidene-L-glycero-D-allo-unde-conate and methyl 3,7:6,10-dianhydro-11-O-benzyl-2,4,5,9-tetradeoxy-9-methylidene-L-glycero-L-talo-undeconate (S5b and S5a): To a stirred solution of aldehyde S4 (165 mg, 0.458 mmol, 1.0 equiv) in toluene (30 mL) at 23° C. were added n-Bu₃SnH (620 L, 2.29 mmol, 5.0 equiv) and AIBN

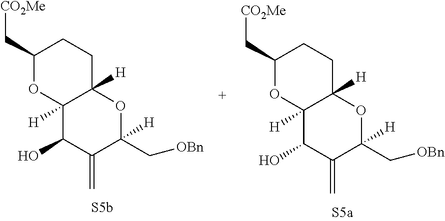 [SMILES: C=C1[C@H](COCc2ccccc2)O[C@H]2CC[C@H](CC(=O)OC)O[C@@H]2[C@@H]1O ; C=C1[C@H](COCc2ccccc2)O[C@H]2CC[C@H](CC(=O)OC)O[C@@H]2[C@H]1O]

(37.6 mg, 0.229 mmol, 0.5 equiv). The flask containing the resulting mixture was transferred into a preheated oil bath (100° C.) and stirred for 1 h before it was allowed to cool to 23° C. The resulting mixture was concentrated under reduced pressure and the residue was passed through a pad of silica gel (hexanes/EtOAc 5:1, v/v→1:1, v/v) furnishing the crude organotin intermediate, which was used in the next step without further purification.

To a stirred solution of the above obtained crude organotin intermediate in CH₂Cl₂ (5 mL) at 23° C. was added p-TsOH·H₂O (44.0 mg, 0.230 mmol, 0.5 equiv). Then, the reaction mixture was stirred for 1 h at 23° C. before it was diluted with NaHCO₃ solution (10 mL, sat. aq.). The layers were separated, and the aqueous layer was extracted with CH₂Cl₂ (3×30 mL). The combined organic extracts were washed with brine (30 mL), dried over Na₂SO₄, and concentrated under reduced pressure. Flash column chromatography (SiO₂, CH₂Cl₂/Et₂O 9:1, v/v→17:3, v/v) of the residue afforded hydroxy alkenes S5b (56.5 mg, 0.156 mmol, 34% yield for the two steps) and S5a (68.1 mg, 0.188 mmol, 41% yield for the two steps) as colorless oils, respectively. S5b: R_{f}=0.50 (SiO₂, CH₂Cl₂/Et₂O 4:1 v/v);

$${[ α ]}_{D}^{23} = - 29.6 ( c = 0.53 , {CHCl}_{3} ) ;$$

FT-IR (film): vₘₐₓ 3459, 2926, 2855, 1736, 1496, 1454, 1437, 1364, 1342, 1288, 1258, 1224, 1196, 1144, 1093, 1075, 1018, 987, 918, 845, 805, 739, 699 cm¹; ¹H NMR (600 MHz, CDCl₃) δ 7.38-7.24 (m, 5H), 5.23-5.17 (m, 1H), 5.11 (d, J=4.4 Hz, 1H), 4.65-4.56 (m, 2H), 4.45 (dt, J=8.3, 4.1 Hz, 1H), 4.33 (t, J=3.7 Hz, 1H), 4.05 (ddd, J=10.7, 8.4, 4.4 Hz, 1H), 3.94 (dddt, J=12.6, 7.9, 5.1, 2.5 Hz, 1H), 3.77-3.64 (m, 4H), 3.49 (dt, J=11.0, 4.1 Hz, 1H), 3.15 (dt, J=9.6, 3.7 Hz, 1H), 2.58 (ddd, J=15.5, 7.6, 4.4 Hz, 1H), 2.44 (dt, J=15.5, 4.8 Hz, 1H), 2.10 (dp, J=11.9, 4.0 Hz, 1H), 1.83 (dq, J=13.6, 2.9 Hz, 1H), 1.59-1.37 (m, 3H)ppm; ¹³C NMR (151 MHz, CDCl₃) δ 171.5, 142.6, 138.2, 128.5, 128.0, 127.8, 118.4, 80.3, 77.8, 74.5, 73.3, 71.1, 70.8, 63.6, 51.9, 40.7, 30.7, 29.3 ppm; HRMS (ESI-TOF) m/z: [M+Na]⁺ Calcd. for C₂₀H₂₆O₆Na⁺ 385.1622; Found 385.1624. S5a: R_{f}=0.45 (SiO₂, CH₂Cl₂/Et₂O 4:1, v/v);

$${[ α ]}_{D}^{23} = - 33.2 ( c = 1.68 , {CHCl}_{3} ) ;$$

FT-IR (film): v. 3462, 2952, 2856, 1735, 1650, 1496, 1454, 1438, 1375, 1349, 1289, 1254, 1198, 1162, 1094, 1082, 1028, 995, 960, 916, 848, 738, 698 cm; ¹H NMR (600 MHz, CDCl₃) δ 7.37-7.27 (m, 5H), 5.35 (dd, J=2.4, 1.4 Hz, 1H), 5.09-5.06 (m, 1H), 4.60 (d, J=12.4 Hz, 1H), 4.57-4.49 (m, 2H), 4.27 (dtd, J=9.7, 2.4, 0.8 Hz, 1H), 3.85-3.75 (m, 2H), 3.69 (s, 3H), 3.52-3.44 (m, 2H), 2.97 (t, J=9.5 Hz, 1H), 2.58 (dd, J=15.3, 7.5 Hz, 1H), 2.44 (dd, J=15.3, 5.5 Hz, 1H), 2.09-2.02 (m, 1H), 1.87-1.81 (m, 1H), 1.59-1.49 (m, 1H), 1.41 (tdd, J=13.4, 11.2, 3.9 Hz, 1H)ppm; ¹³C NMR (151 MHz, CDCl₃) δ 171.4, 143.2, 138.1, 128.6, 127.9, 127.8, 111.1, 84.2, 79.0, 74.1, 73.3, 70.9, 70.2, 69.6, 52.0, 40.7, 30.5, 29.4 ppm; HRMS (ESI-TOF) m/z: [M+Na]⁺ Calcd. for C₂₀H₂₆O₆Na⁺ 385.1622; Found 385.1624.
Methyl 3,7:6,10-dianhydro-11-O-benzyl-8-O-[tert-butyl(dimethyl)silyl]-2,4,5,9-tetradeoxy-9-methylidene-glycero-D-allo-undeconate (S12): To a stirred solution of hydroxy alkene S5b (17.2 mg, 0.0472 mmol, 1.0 equiv) and 2,6-lutidine (27.3 μL, 0.236 mmol, 5.0 equiv) in CH₂Cl₂ (5 mL)

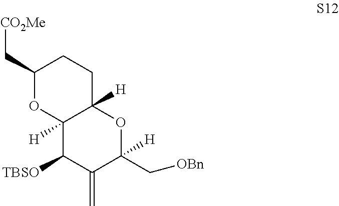 [SMILES: C=C1[C@H](COCc2ccccc2)O[C@H]2CC[C@H](CC(=O)OC)O[C@@H]2[C@@H]1O[Si](C)(C)C(C)(C)C]

at −78° C. was added TBSOTf (32.6 μL, 0.142 mmol, 3.0 equiv). The resulting mixture was warmed to −20° C. and stirred for 1 h before it was quenched by addition of NaHCO₃ solution (5 nL, sat. aq.). The layers were separated, and the aqueous layer was extracted with CH₂Cl₂ (3×10 mL). The combined organic extracts were dried over Na₂SO₄ and concentrated under reduced pressure. Flash column chromatography (SiO₂, hexanes/EtOAc 10:1, v/v→2:1, v/v) of the residue afforded silyl ether S12 (16.0 mg, 0.0335 mmol, 71% yield) as a colorless oil. S12: R_{f}=0.80 (SiO₂, hexanes/EtOAc 1:1, v/v);

$${[ α ]}_{D}^{23} = - 41.5 ( c = 0.2 , EtOAc ) ;$$

FT-IR (film): vₘₐₓ 2952, 2928, 2855, 1742, 1455, 1437, 1361, 1252, 1195, 1150, 1104, 1016, 918, 835, 777, 738 cm⁻¹; ¹H NMR (600 MHz, CDCl₃): δ 7.37-7.27 (m, 5H), 4.96 (s, 2H), 4.67-4.51 (m, 2H), 4.43 (dd, J=9.7, 3.4 Hz, 1H), 4.25 (d, J=2.7 Hz, 1H), 4.18 (dd, J=11.1, 9.7 Hz, 1H), 3.84 (dddd, J=10.8, 8.2, 4.9, 2.1 Hz, 1H), 3.67 (s, 3H), 3.64-3.60 (m, 1H), 3.27 (dd, J=11.1, 3.4 Hz, 114), 3.04 (dd, J=9.4, 2.71 Hz, 1H), 2.51 (dd, J=15.2, 8.3 Hz, 1H), 2.38 (dd, J=15.2, 4.9 Hz, 1H), 2.07 (dt, J=12.0, 3.9 Hz, 1H), 1.78 (ddd, J=13.4, 3.8, 1.9 Hz, 1H), 1.49 (tdd, J=12.4, 11.0, 4.0 Hz. 1H), 1.33 (tdd, J=13.4, 11.2, 3.8 Hz, 1H), 0.80 (s, 9H), −0.01 (s, 3H), −0.04 (s, 3H)ppm; ¹³C NMR (151 MHz, CDCl₃): δ 171.8, 144.5, 138.6, 128.5, 128.0, 127.7, 115.1, 80.8, 78.6, 74.1, 73.1, 72.5, 70.2, 63.1, 51.8, 41.0, 30.8, 29.4, 25.9, 18.4, −4.5, −4.9 ppm; HRMS (ESI-TOF) m/z: [M+Na]⁺ Calcd. for C₂₆H₄₀O₆SiNa⁺ 499.2486; Found 499.2479.
Methyl 3,7:6,10-dianhydro-11-O-benzyl-8-O-[tert-butyl(dimethyl)silyl]-2,4,5-trideoxy-L-threo-D-allo-undeconate (S6): Through a stirred solution of alkene S12 (8.2 mg, 0.017 mmol, 1.0 equiv) in

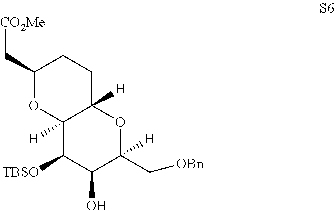 [SMILES: COC(=O)C[C@H]1CC[C@@H]2O[C@@H](COCc3ccccc3)[C@@H](O)[C@@H](O[Si](C)(C)C(C)(C)C)[C@H]2O1]

MeOH (2 mL) at −78° C. was bubbled O₃. After 1 min (when the solution turned light blue), the resulting mixture was treated with NaBH₄ (3.3 mg, 0.085 mmol, 5.0 equiv). The mixture was allowed to warm to 23° C. and stirred for 1 h before it was diluted with NaHCO₃ solution (5 mL, sat. aq.). The layers were separated, and the aqueous layer was extracted with CH₂Cl₂ (3×10 mL). The combined organic extracts were dried over Na₂SO₄ and concentrated under reduced pressure. Flash column chromatography (SiO₂, hexanes/EtOAc 5:1, v/v/v→1:1, v/v) of the residue afforded hydroxy ester S6 (6.3 mg, 0.013 mmol, 77% yield) as a colorless oil. S6: R_{f}=0.50 (SiO₂, hexanes/EtOAc 1:1, v/v);

$${[ α ]}_{D}^{23} = - 85. ( c = 0.1 , {CHCl}_{3} ) ;$$

FT-IR (film): vₘₐₓ 3449, 2928, 2855, 1742, 1460, 1438, 1346, 1250, 1187, 1122, 1076, 1017, 992, 833, 778, 698 cm⁻¹; ¹H NMR (600 MHz, C₆D₆): δ 7.37-7.26 (m, 2H), 7.16-6.95 (m, 3H), 4.46 (dd, J=85.1, 12.2 Hz, 2H), 4.36-4.30 (m, 1H), 4.07 (dd, J=10.4, 4.9 Hz, 1H), 3.98 (dd, J=10.4, 7.7 Hz, 1H), 3.93 (s, 1H), 3.68 (dddd, J=10.6, 6.8, 4.7, 2.0 Hz, 1H), 3.59 (ddd, J=9.0, 6.5, 2.7 Hz, 1H), 3.44 (td, J=10.2, 4.6 Hz, 1H), 3.39 (s, 3H). 2.66 (dd, J=9.4, 2.51 Hz, 1H), 2.41-2.30 (m, 2H), 2.05 (dd, J=15.3, 4.714 Hz, 1H), 1.89 (dt, J=13.6, 3.7 Hz, 1H), 1.33-1.26 (m, 2H), 0.97 (s, 9H), 0.13 (s, 3H), 0.12 (s, 3H)ppm; ¹³C NMR (151 MHz, C₆D₆): δ 170.9, 139.1, 128.6, 128.4, 127.7, 79.5, 75.1, 74.5, 73.3, 72.4, 69.9, 68.2, 63.5, 51.1, 40.8, 30.7, 29.6, 26.3, 18.7, −4.1, −4.7 ppm; HRMS (ESI-TOF) mvz: [M+Na]⁺ Calcd. for C₂₃H₄₀O₇SiNa⁺ 503.2436: Found 503.2432.

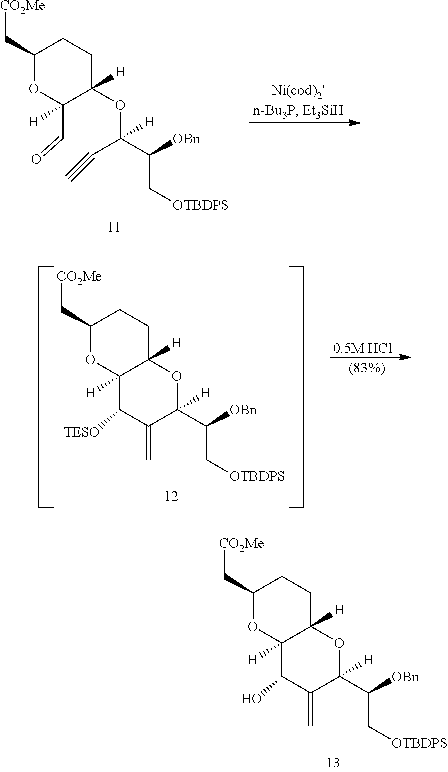

Methyl 3,7:6,10-dianhydro-11-O-benzyl-12-O-[tert-butyl(diphenyl)silyl]-2,4,5,9-tetradeoxy-9-methylidene-8-O-(triethylsilyl)-L-erythro-L-talo-dodeconate and methyl 3,7:6,10-dianhydro-11-

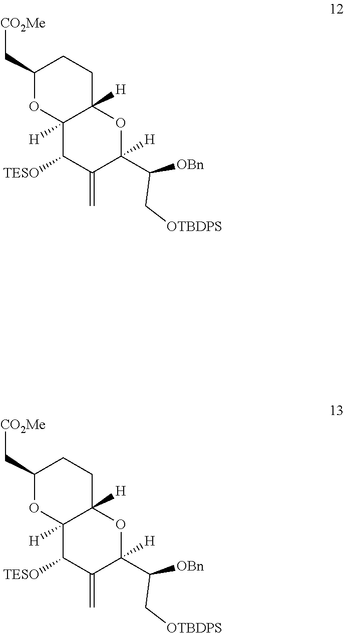

O-benzyl-12-O-[tert-butyl(diphenyl)silyl]-2,4,5,9-tetradeoxy-9-methylidene-L-erythro-L-talo-dodeconate (12 and 13): To a stirred solution of aldehyde 11 (560 mg, 0.90 mmol, 1.0 equiv) and Et₃SiH (1.0 mL, 6.3 mmol, 7.0 equiv) in THF (100 mL) at 23° C. was added a premixed solution of n-Bu₃P (67 μL, 0.27 mmol, 0.3 equiv) and Ni(cod)₂ (37 mg, 0.13 mmol, 0.15 equiv) in THF (10 mL) dropwise. The resulting mixture was stirred for 5 h at 23° C. before it was diluted with aq. HCl solution (0.5M, 20 mL). The reaction mixture was further stirred for 3 h before it was quenched by addition of NaHCO₃ solution (20 mL, sat. aq.). The layers were separated, and the aqueous layer was extracted with EtOAc (3×50 mL). The combined organic extracts were dried over Na₂SO₄ and concentrated under reduced pressure. Flash column chromatography (SiO₂, hexanes/EtOAc 10:1, v/v→2:1, v/v) of the residue afforded alcohol 13 (470 mg, 0.743 mmol, 83% yield) a colorless oil (Note: A pure sample of 12 for analysis was obtained by quenching the reaction with sat. aq. NaHCO₃ solution after its first stage). 12: R_{f}=0.60 (SiO₂, hexanes/EtOAc 4:1, v/v);

$${[ α ]}_{D}^{23} = + 8.3 ( c = 0.66 , {CHCl}_{3} ) ;$$

FT-IR (film): vₘₐₓ 2952, 2933, 2875, 2859, 1742, 1428, 1239, 1195, 1150, 1103, 1088, 999, 961, 913, 824, 739, 701 cm¹; ¹H NMR (600 MHz, CDCl₃) δ 7.73-7.67 (m, 41H), 7.46-7.41 (m, 2H), 7.41-7.35 (m, 4H), 7.32-7.26 (m, 5H), 5.42 (t, J=2.2 Hz, 1H), 5.07-5.04 (m, 1H), 4.68 (d, J=11.1 Hz, 1H), 4.47 (d, J=11.1 Hz, 1H), 4.45-4.39 (m, 1H), 4.35 (d, J=6.5 Hz, 1H), 3.92-3.87 (m, 1H), 3.79-3.75 (m, 3H), 3.68 (s, 3H), 3.42 (ddd, J=10.8, 9.3, 4.0 Hz, 1H), 2.92 (t, J=9.3 Hz, 1H), 263 (dd, J=15.6, 6.0 Hz, 1H), 239 (dd, J=15.5, 6.8 Hz, 1H), 1.87-1.72 (m, 2H), 1.46-1.37 (m, 1H), 1.37-1.28 (m, 1H), 1.07 (s, 9H), 0.93 (t, J=8.0l-Hz, 9H), 0.62-0.57 (m, 6H)ppm; ¹³C NMR (151 MHz, CDCl₃) δ 171.4, 144.1, 138.5, 135.79, 135.75, 134.9, 133.6, 133.4, 129.90, 129.87, 128.4, 127.9, 127.8, 127.7, 113.0, 84.4, 81.0, 79.1, 73.7, 73.4, 72.2, 71.6, 64.0, 51.8, 40.7, 30.7, 29.9, 27.01, 26.99, 19.3, 7.8, 5.0 ppm; HRMS (ESI-TOF) m/z: [M±Na]⁺ Calcd. for C₄₃H₆₀O₇SiO₂Na⁺ 767.3770; Found 767.3766, 13: R_{f}=0.40 (SiO₂, hexanes/ELOAc 2:1, v/v);

$${[ α ]}_{D}^{23} = - 8.3 ( c = 0.58 , EtOAc ) ;$$

FT-IR (film): vₘₐₓ 3472, 2930, 2857, 1738, 1428, 1197, 1091, 997, 823, 801, 740, 701 cm¹; ¹H NMR (600 MHz, CDCl₃) δ 7.76-7.61 (m, 4H), 7.44-7.41 (m, 2H), 7.39-7.36 (m, 4H), 7.33-7.24 (m, 514), 5.44 (dd, J=2.3, 1.7 Hz, 1H), 5.13 (d, J=1.7 Hz, 1H), 4.66 (d, J=11.4 Hz, 1H), 4.49 (d, J=11.4 Hz, 1H), 4.33 (d, J=7.2 Hz, 1H), 4.30 (d, J=9.6 Hz, 1H), 3.87 (ddd, J=7.2, 5.9, 4.7 Hz, 1H), 3.82-3.74 (m, 3H), 3.69 (s, 3H), 3.36 (ddd, J=10.8, 9.3, 4.3 Hz, 1H), 2.93 (t, J=9.5 Hz, 1H), 2.58 (dd, J=15.4, 7.4 Hz, 1H), 2.48-2.38 (m, 2H), 1.80-1.73 (m, 2H), 1.48-1.35 (m, 1H), 1.34-1.24 (m, 1H), 1.07 (s, 911)ppm; ¹³C NMR (151 MHz, CDCl₃) δ 171.4, 142.2, 138.3, 135.78, 135.75, 133.5, 133.4, 129.89, 129.88, 128.4, 128.1, 127.9, 127.8, 112.4, 84.4, 79.8, 79.0, 74.0, 730.2, 71.1, 70.7, 63.9, 51.9, 40.8, 30.5, 29.5, 27.01, 26.98, 19.3 ppm; HRMS (ESI-TOF) m/z: [M+Na]⁺ Calcd. for C₃₇H₄₆O₇SiNa⁺ 653.2905; Found 653.2905.
Methyl 3,7:6,10-dianhydro-11-O-benzyl-12-O-[tert-butyl(diphenyl)silyl]-2,4,5,9-tetradeoxy-9-methylidene-L-glycero-L-talo-dodec-8-ulosonate (14): To a stirred solution of alcohol 13 (730 mg, 1.16 mmol 1.0 equiv) in CH₂Cl₂ (20 mL) at 0° C. was added Dess-Martin periodinane (1.23 g,

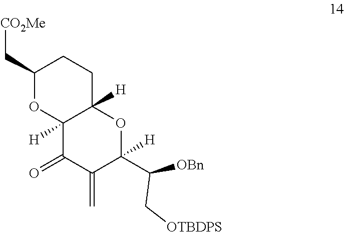

2.90 mmol, 2.5 equiv). The resulting mixture was warmed to 23° C. and stirred for 1.5 h before it was diluted with NaHCO₃ solution (20 mL, sat. aq.) and Na₂S₂O₃ solution (15 mL, sat. aq.). The resulting mixture was vigorously stirred for 1 h. Then, the layers were separated, and the aqueous layer was extracted with CH₂Cl₂ (3×30 mL). The combined organic extracts were dried over Na₂SO₄, and concentrated under reduced pressure. Flash column chromatography (SiO₂, hexanes/EtOAc 5:1, v/v→1:1, v/v) of the residue afforded enone 14 (666 mg, 1.06 mmol, 91% yield) as a colorless oil. 14: R_{f}=0.50 (SiO₂, hexanes/EtOAc 2:1, v/v);

$${[ α ]}_{D}^{23} = - 15.6 ( c = 0.4 , EtOAc ) ;$$

FT-IR (film): vₘₐₓ 2932, 2859, 1738, 1717, 1428, 1196, 1111, 1079, 824, 740, 702 cm⁻¹; ¹H NMR (600 MHz, C₆D₆): δ 7.84-7.68 (m, 4H), 7.25-7.17 (m, 10H), 7.10-7.05 (m, 1H), 6.03 (s, 1H), 5.09 (s, 1H), 4.71 (d, J=7.4 Hz, 1H), 4.50 (d, J=11.6 Hz, 1H) 4.22 (d, J=11.5 Hz, 1H), 3.86-3.77 (m, 2H), 3.67 (dtd, J=11.3, 6.4, 2.2 Hz, 1H), 3.61 (dt, J=7.3, 4.6 Hz, 1H), 3.60-3.54 (m, 2H), 3.32 (s, 3H), 2.63 (dd, J=15.6, 6.6 Hz, 1H), 2.22 (dd, J=15.6, 6.3 Hz, 1H), 1.67 (dt, J=10.5, 3.41 Hz, 1H), 1.41 (ddt, J=13.5, 4.9, 2.6 Hz, 1H), 1.35-1.27 (m, 1H), 1.14 (s, 9H), 1.05 (tdd, J=13.1, 11.3, 3.9 Hz, 1H)ppm; ¹³C NMR (151 MHz, CD) d 192.5, 170.8, 142.9, 138.6, 136.1, 136.0, 133.72, 133.66, 130.2, 130.1, 128.6, 127.8, 122.9, 83.6, 80.7, 77.2, 74.4, 73.1, 70.9, 63.3, 51.3, 40.6, 30.6, 30.0, 27.0, 19.4 ppm; HRMS (ESI-TOF) m/z: [M+—Na]⁺ Calcd. for C₃₇H₄₄O₇SiNa⁺ 651.2749; Found 651.2751.
Methyl 3,7:6,10-dianhydro-11-O-benzyl-12-O-[tert-butyl(diphenyl)silyl]-2,4,5-trideoxy-L-arabino-D-allo-dodeconate (15): Through a stirred solution of enone 14 (117 mg, 0.186 mmmol, 1.0 equiv) in MeOH (15 mL) at −78° C. was bubbled O₃. After about 5 min (when the solution turned

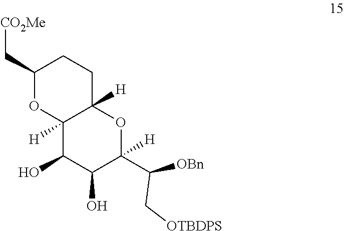

light blue), the resulting mixture was treated with NaBH₄ (35.2 mg, 0.930 mmol, 5.0 equiv), the resulting mixture was allowed to warm to 23° C. and stirred for 1.5 h before it was diluted with NaHCO₃ solution (15 mL, sat. aq.). The layers were separated, and the aqueous layer was extracted with CH₂Cl₂ (3×30 mL). The combined organic extracts were dried over Na₂SO₄ and concentrated under reduced pressure. Flash column chromatography (SiO₂, hexanes/EtOAc 2:1, v/v→1:3, v/v) of the residue afforded diol 15 (83.8 mg, 0.132 mmol, 71% yield) as a white foam. 15: R_{f}=0.50 (SiO₀, hexanes/EtOAc 1:1, v/v);

$${[ α ]}_{D}^{23} = - 4.5 ( c = 0.4 , EtOAc ) ;$$

FT-IR (film): v. 3454, 2931, 2858, 1740, 1428, 1345, 1257, 1196, 1112, 1070, 1027, 999, 943, 801, 742, 702 cm⁻¹; ¹H NMR (600 MHz, CDCl₁): δ 7.83-7.63 (m, 4H), 7.46-7.34 (m, 10H), 7.33-7.29 (m, 1H), 4.92 (d, J=10.9 Hz, 1H), 4.77 (d, J=6.9 Hz, 1H), 4.70 (ddd, J=10.0, 4.9, 2.1 Hz, 1H), 4.60 (d, J=10.91 Hz, 1H), 4.23 (t, J=2.8 Hz, 1H), 4.03 (dd, J=10.0, 6.01 Hz, 1H), 3.98 (dd, J=11.7, 2.0 Hz, 1H), 3.93 (td, J=6.5, 2.8 Hz, 1H), 3.89-3.83 (m, 2H), 3.69 (s, 3H), 3.41 (td, J=10.1, 4.4 Hz, 1H), 3.08 (dd, J=9.6, 2.81 Hz, 1H), 2.64 (dd, J=15.6, 7.21 Hz, 1H), 2.44 (dd, J=15.6, 5.814 Hz, 1H), 2.33 (s, 1H), 1.82-1.73 (m, 21-1), 1.44-1.31 (m, 2H), 1.07 (s, 9H)ppm; ¹³C NMR (151 MHz, CDCl₃): δ 171.5, 137.5, 135.83, 1350.79, 133.4, 133.3, 129.89, 129.86, 128.8, 128.3, 127.9, 80.7, 78.9, 74.6, 72.8, 71.0, 70.5, 70.1, 64.6, 63.9, 51.9, 40.7, 30.6, 29.2, 27.0, 19.4 ppm; HRMS (ESI-TOF) m/z: [M+Na]⁺ Calcd. for C₃₆H₄₆O₈SiNa⁺ 657.2854; Found 657.2852.

### Preparation of Alcohol 16:

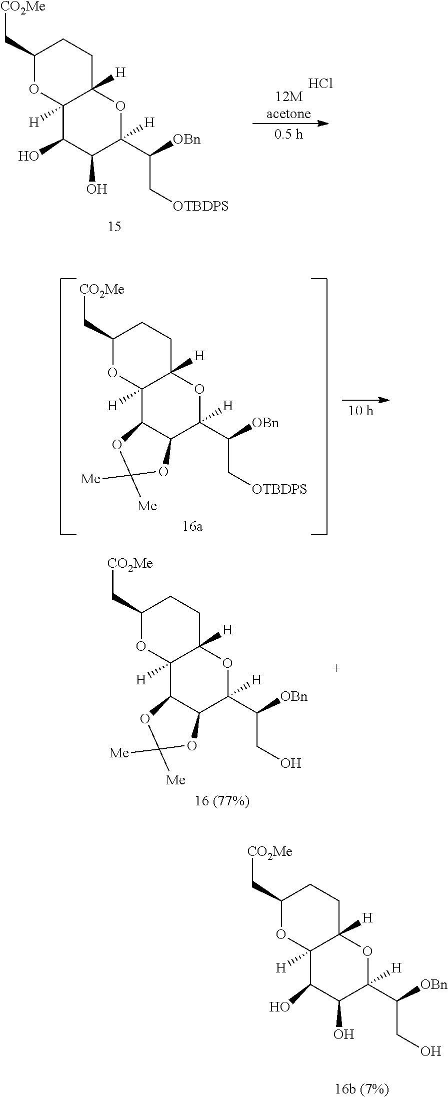

Methyl 3,7:6,10-dianhydro-11-O-benzyl-2,4,5-trideoxy-8,9-O-(1-methylethylidene)-L-arabino-D-allo-dodeconate (16): To a stirred solution of diol 15 (337 mg, 0.531 mmol, 1.0 equiv) in acetone/MeOH (20 mL, 4:1, v/v) at 0° C. was added aq. HCl solution (12M, 2.5 mL). The resulting

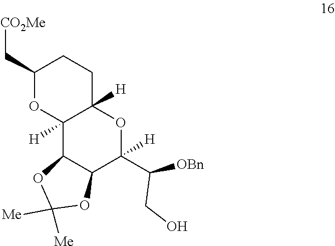 [SMILES: COC(=O)C[C@H]1CC[C@@H]2O[C@@H]([C@H](CO)OCc3ccccc3)[C@H]3OC(C)(C)O[C@H]3[C@H]2O1]

mixture was allowed to warm to 23° C. and stirred for 10 h before it was quenched by addition of NaHCO₃ solution (20 mL, sat. aq.). The layers were separated, and the aqueous layer was extracted with CH₂Cl₂ (3×30 mL). The combined organic extracts were dried over Na₂SO₄ and concentrated under reduced pressure. Flash column chromatography (SiO₂, hexanes/EtOAc 2:1-1:10, v/v, then EtOAc:MeOH 20:1, v/v→1:5, v/v) of the residue afforded alcohol 16 (178 mg, 0.409 mmol, 77% yield) as a colorless oil and triol 16b (14.8 mg, 0.0372 mmol, 7% yield) as a white foam (Note: Intermediate 16a was obtained as a colorless oil when the reaction was quenched after 1 h). 16a: R_{f}=0.50 (SiO₂, hexanes/EtOAc 3:1, v/v);

$${[ α ]}_{D}^{23} \text{=+} 2.5 ( c = 0.5 , {CHCl}_{3} ) ;$$

FT-IR (film): vₘₐₓ 2929, 2857, 1740, 1455, 1428, 1256, 1208, 1088, 990, 823, 702 cm⁻¹; ¹H NMR (600 MHz, CDCl₃): δ 7.75-7.69 (m, 4H), 7.44-7.38 (m, 2H), 7.38-7.30 (m, 8H), 7.31-7.26 (m, 1H), 4.73 (d, J=11.2 Hz, 1H), 4.69 (dd, J=8.5, 1.4 Hz, 1H), 4.56 (d, J=11.2 Hz, 1H), 4.52 (dd, J=8.6, 3.1 Hz, 1H), 3.95 (brs, 1H), 3.93 (brs, 1H), 3.89-3.79 (m, 3H), 3.67 (s, 41H), 3.49 (dd, J=10.2, 3.2 Hz, 1H), 2.72 (dd, J=16.1, 6.81 Hz, 1H), 2.42 (dd, J=16.1, 6.2 Hz, 1H), 2.09-2.01 (m, 1H), 1.75 (dd, P=13.2, 2.8 Hz, 1H), 1.56 (s, 3H), 1.53-1.47 (m, 1H), 1.46-1.38 (m, 1H), 1.38 (s, 3H), 1.06 (s, 9H)ppm; ¹³C NMR (151 MHz, CDCl₃): δ 171.7, 138.8, 135.80, 135.77, 133.81, 133.75, 129.7, 129.7, 128.4, 128.1, 1270.74, 127.68, 109.6, 78.9, 76.6, 752, 74.0, 72.9, 71.3, 67.9, 66.0, 62.6, 51.8, 40.5, 30.8, 30.2, 26.9, 26.3, 24.1, 19.5 ppm; HIRMS (ESI-TOF) m/z: [M±Na]⁺ Calcd. for C₃₉H₅₀O₈SiNa⁺ 697.3167; Found 697.3169, 16: R_{f}=0.20 (SiO₂, hexanes/EtOAc 1:1, v/v);

$${[ α ]}_{D}^{23} = - 33.2 ( c = 0.36 , {CHCl}_{3} ) ;$$

FT-IR (film): vₘₐₓ 3505, 2934, 2869, 1737, 1455, 1438, 1380, 1257, 1207, 1104, 1078, 1027, 990, 741 cm¹; H NMR (600 MHz, CDCl₃): δ 7.37-7.27 (m, 5H), 4.64 (AB quart, J=11.2 Hz, 2H), 4.59 (dd, J=8.5, 1.5 Hz, 1H), 4.53 (dd, J=8.5, 3.1 Hz, 1H), 3.87-3.80 (m, 3H), 3.79-3.72 (m, 2H), 3.70-3.62 (m, 4H), 3.50 (dd, J=10.2, 3.1 Hz, 1H), 2.71 (dd, J=16.2, 6.8 Hz, 1H), 2.42 (dd, J=16.1, 6.1 Hz, 1H), 2.17 (dd, J=8.7, 4.1 Hz, 1H), 2.09-2.00 (m, 114), 1.77 (ddt, J=13.5, 4.5, 2.4 Hz, 1H), 1.56 (s, 4H), 1.45-1.39 (m, 1H), 1.37 (s, 3H)ppm; ¹³C NMR (151 MHz, CDCl₃): δ 171.6, 138.2, 128.6, 128.2, 128.1, 109.9, 77.8, 76.3, 75.2, 73.9, 73.0, 71.3, 67.0, 66.1, 62.1, 51.8, 40.4, 30.7, 30.1, 26.2, 24.1 ppm; HRMS (ESI-TOF) m/z: [M+Na]⁺ Calcd. for C₂₃H₃₂O₈Na⁺ 459.1989; Found 459.1995, 16b: R_{f}=0.30 (SiO₂, 100% EtOAc);

$${[ α ]}_{D}^{23} = + 4.1 ( c = 0.6 , {CHCl}_{3} ) ;$$

FT-IR, film): vₘₐₓ, 3438, 2929, 2870, 1736, 1455, 1438, 1346, 1292, 1199, 1102, 1068, 751 cm⁻¹; ¹H NMR (600 MHz, CDCl₃): δ 7.38-7.29 (m, 5H), 4.73 (d, J=10.9 Hz, 1H), 4.67-4.60 (m, 2H), 4.40 (d, J=6.9 Hz, 1H), 4.25 (t, J=2.9 Hz, 1H), 4.07 (dd, J=9.9, 5.9 Hz, 1H), 3.96 (td, J=6.3, 2.81 Hz, 1H), 3.93-3.86 (m, 2H), 3.82-3.76 (m, 1H), 3.69 (s, 3H), 3.56 (td, J=10.1, 4.4 Hz, 1H), 3.12 (dd, J=9.6, 2.81 Hz, 1H), 2.62 (dd, J=15.6, 7.3 Hz, 1H), 244 (dd, J=15.6, 5.6 Hz, 1H), 2.39 (s, 1H), 2.03-1.98 (m, 1H), 1.93 (d, J=4.7 Hz, 1H), 1.88-1.79 (m, 1H), 1.53-1.38 (m, 2H)ppm; ¹³C NMR (151 MHz, CDCl₃): δ 171.5, 137, 129.0, 128.6, 128.4, 79.6, 78.7, 74.6, 72.4, 70.72, 70.68, 70.2, 65.0, 61.1, 519, 40.6, 30.6, 29.3 ppm; HRMS (ESI-TOF) m/z: [M+Na]⁺ Calcd. for C₂₀H₂₈O₈Na⁺ 419.1676; Found 419.1674.
Methyl [(1S,2S,4R,7S,9R,10S,12S,14R,16R)-12-(2-{(2S,5S)-5-[(3R,5R)-3-hydroxy-6-iodo-5-methylhept-6-en-1-yl]-4-methylidenetetrahydrofuran-2-yl}ethyl)-3,8,11,15,17-penta-

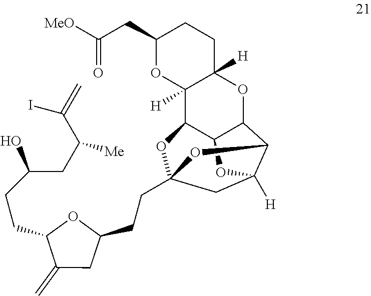

oxapentacyclo[10.4.1.0^{2,7}.0^{9,16}.0^{10,14}heptadec-4-yl]acetate (21): continuous flow process was set up for the cleavage of the benzyl protecting group within 19 and C12-epi-19. To a stirred solution of 19/C12-epi-19 (100 mg, 0.128 mmol, 1.0 equiv, 2:1 dr) in MeCN (100 mL) at 23° C. were added 2,3-dichloro-5,6-dicyano-1,4-benzoquinone (DDQ, 81.4 mg, 0.358 mmol, 2.8 equiv) and 2,6-di-tert-butyl-4-methylphenol (BHT, 5.64 mg, 0.0256 mmol, 0.2 equiv). The resulting mixture was stirred for 5 min before it was transferred into a 60 mL syringe (each time using 50 mL) and placed on a Fisherbrand™ 7801001 syringe pump. The reaction mixture was pumped (18 mL/h) through a FPE loop ( 1/16″ OD×0.030″ ID×70 mm L) to be irradiated by a blue LED lamp (Kessil PR160-390, 390 nm, power=100%) at a distance of 15 cm. The draining reaction mixture at the outlet of the FPE loop was collected in a brown round-bottom flask until completion of the continuous flow process. To the stirred collected reaction mixture was added p-TsOHH₂O(5.00 mg, 0.0257 mmol, 0.30 equiv) and the mixture was stirred for 5 min before it was diluted with NaHCO₃ solution (20 ml, sat. aq.) and EtOAc (30 mL). The layers were separated, and the aqueous layer was extracted with EtOAc (4×50 mL). The combined organic extracts were washed with brine (30 mL), dried over Na₂SO₄ and concentrated under reduced pressure. Flash column chromatography (SiO₂, hexanes/EtOAc 2:1, v/v→1:10, v/v; then EtOAc/MeOH 50:1, v/v→10:1, v/v) of the residue afforded alcohol 21 (40.3 mg, 0.0597 mmol, 70% yield from 19), C12-epi-20 (contaminated with trace amount of unreacted 20) (19.8 mg, 0.0286 mmol, 67% yield from C12-epi-19), and recovered 19/C12-epi-19 (19.0 mg, 0.0243 mmol, 19%, ~2:1 dr) as colorless oils. 21: R_{f}=0.60 (SiO₂, 100% EtOAc);

$${[ α ]}_{D}^{23} = - 59. ( c = 1. , {CHCl}_{3} ) ;$$

FT-IR (film): vₘₐₓ 3450, 2930, 2866, 1738, 1617, 1437, 1336, 1290, 1210, 1190, 1155, 1133, 1076, 1013, 903, 830 cm⁻¹; ¹H NMR (600 MHz, CDCl₃): δ 6.23 (t, J=1.0 Hz, 1H), 5.75 (d, J=1.3 Hz, 1H), 5.01 (d, J=2.2 Hz, 1H), 4.86 (d, J=2.2 Hz, 1H), 4.68 (t, J=4.7 Hz, 1H), 4.61 (t, J=4.5 Hz, 1H), 4.41 (ddd, J=13.5, 5.1, 2.8 Hz, 2H), 4.27 (td, J=10.2, 4.4 Hz, 1H), 4.20 (dd, J=6.6, 4.6 Hz, 1H), 4.16-4.10 (m, 1H), 4.06 (dd, J=6.6, 30.91 Hz, 1H), 3.85-3.77 (m, 1H), 3.67 (s, 3H), 3.61-3.49 (m, 1H), 2.93 (dd, J=9.6, 1.91 Hz, 1H), 2.74-2.67 (m, 1H), 2.67-2.60 (m, 2H), 2.39 (dd, J=15.9, 6.01 Hz, 1H), 2.32-2.25 (m, 1H), 2.17-2.07 (m, 4H), 1.96 (dd, 1=13.4, 5.1 Hz, 1H), 1.87 (dddd, J=13.2, 11.3, 7.8, 4.1 Hz, 1H), 1.80 (dt, J=9.6, 2.4 Hz, 1H), 1.77-1.68 (m, 2H), 1.67-1.59 (m, 2H), 1.54 (tt, J=13.2, 7.7 Hz, 1H), 1.46-1.34 (m, 3H), 1.29-1.22 (m, 1H), 0.98 (d, J=6.6 Hz, 3H)ppm; ¹³CNMR (151 MHz, CDCl₃): δ 171.7, 151.4, 125.2, 123.6, 110.4, 105.2, 82.2, 81.1, 79.4, 78.1, 77.3, 76.7, 74.54, 74.51, 74.1, 68.6, 68.4, 51.8, 47.3, 43.7, 42.7, 40.6, 38.9, 35.0, 34.4, 31.2, 30.7, 30.1, 29.6, 22.6 ppm; HRMS (ESI-TOF) m/z: [M+Na]⁺ Calcd. for C₃₀H₄₃IO₉Na⁺ 697.1844; Found 697.1824.

### Epimerization of C12-epi-20:

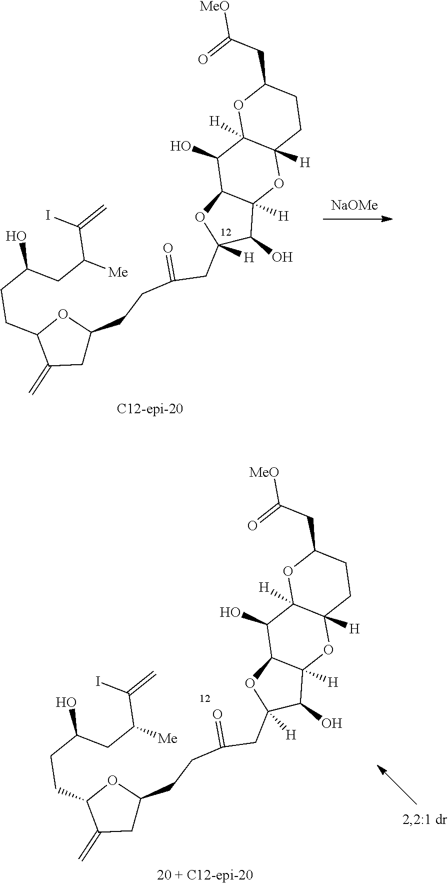 [SMILES: C=C(I)C(C)C[C@H](O)CCC1O[C@@H](CCC(=O)C[C@@H]2O[C@H]3[C@@H](O)[C@H]4O[C@@H](CC(=O)OC)CC[C@@H]4O[C@H]3[C@H]2O)CC1=C ; C=C(I)[C@H](C)C[C@H](O)CC[C@@H]1O[C@@H](CCC(=O)C[C@H]2O[C@H]3[C@@H](O)[C@H]4O[C@@H](CC(=O)OC)CC[C@@H]4O[C@H]3[C@H]2O)CC1=C ; "NaOMe"]

Methyl (20S)-3,7:6,10:9,12:17,20-tetraanhydro-2,4,5,13,15,16,18,19-octadeoxy-20-[(3R,5R)-3-hydroxy-6-iodo-5-methylhept-6-en-1-yl]-19-methylidene-D-glvcero-D-galacto-D-allo-icos-14-ulosonate and methyl (20S)-3,7:6,10:9,12:17,20-tetraanhydro-2,4,5,13,15,16,18,19-octadeoxy-20-[(3R,5R)-3-hydroxy-6-iodo-5-methylhept-6-en-1-yl]-19-methylidene-D-glycero-L-altro-D-allo-icos-14-ulosonate (20 and C12-epi-20): To a stirred solution of C12-epi-20 (65.7 mg, 0.0949 mmol, 1.0 equiv) in MeOH (5 mL) at 23° C. was added NaOMe (25 wt. % in MeOH, 500 μL). The resulting

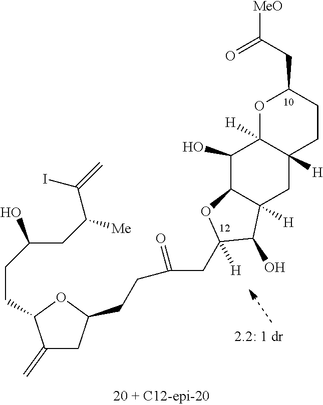 [SMILES: C=C(I)[C@H](C)C[C@H](O)CC[C@@H]1O[C@@H](CCC(=O)C[C@H]2O[C@H]3[C@@H](O)[C@H]4O[C@@H](CC(=O)OC)CC[C@@H]4C[C@H]3[C@H]2O)CC1=C]

mixture was stirred for 7 h before it was diluted with NH₄Cl solution (10 mL, sat. aq.). The layers were separated, and the aqueous layer was extracted with CH₂Cl₂ (4×10 mL). The combined organic extracts were dried over Na₂SO₄ and concentrated under reduced pressure. Flash column chromatography (SiO₂, EtOAc/MeOH 50:1, v/v→10:1, v/v) of the residue afforded 20/C12-epi-20 (59.1 mg, 0.0854 mmol, 90%, 2.2:1 dr) as colorless oils. 20/C12-epi-(mixture of isomers, 2.2:1 dr): R_{f}=0.30 (SiO₂, 100% EtOAc);

$${[ α ]}_{D}^{23} = - 87.4 ( c = 0.56 , {CHCl}_{3} ) ;$$

FT-IR (film): vₘₐₓ 3438, 2931, 2867, 1738, 1617, 1437, 1290, 1260, 1194, 1152, 1072, 1016, 894, 797 cm⁻¹; ¹H NMR (CDCl₃, 600 MHz) δ 6.43 (d, J=0.7 Hz, 0.3H), 635 (d, J=0.6 Hz, 0.67H), 5.77 (d, J=1.3 Hz, 0.69H), 5.76 (d, J=1.3 Hz, 0.35H), 5.65 (d, J=3.2 Hz, 0.291H), 5.42 (d, J=12.1 Hz, 0.2914), 5.14 (d, J=11.7 Hz, 0.681), 5.03 (q, J=2.2 Hz, 0.32H), 5.02 (q, J=2.2 Hz, 0.72H), 4.92 (d, J=3.7 Hz, 0.66H), 4.86 (apd, J=2.2 Hz, 1.30H), 4.56 (dd, J=11.3, 3.1 Hz, 0.34H), 4.41 (dd, J=9.1, 4.7 Hz, 0.77H), 4.39-4.31 (m, 2H), 4.24 (d, J=4.5 Hz, 0.40H), 4.16-4.05 (m, 3.89H), 4.02 (td, J=10.0, 4.9 Hz, 0.85H), 3.97 (ddd, J=11.7, 4.7, 3.0 Hz, 0.831H), 3.91-3.84 (m, 1H), 3.67 (d, J=6.1 Hz, 3H), 3.60 (brs, 1H), 3.05-3.03 (m, 1.23H), 2.94 (dd, J=17.4, 8.3 Hz, 0.79H), 2.84 (ddd, J=17.6, 10.0, 4.5 Hz, 0.35H), 2.77-2.55 (m, 4.65H), 2.54-2.22 (m, 4H), 2.17 (q, J=4.9 Hz, 1H), 2.05 (ddtt, J=13.6, 10.4, 6.8, 3.0 Hz, 1H), 1.91-1.66 (m, 6H), 1.58-1.48 (m, 1H), 1.44-1.43 (m, 1H), 1.27-1.23 (m, 1H), 0.97-0.94 (m, 3H)ppm; BC NMR (151 MHz, CDCl₃) δ 208.9, 207.5, 171.49, 171.46, 150.3, 150.1, 126.2, 125.8, 123.43, 123.42, 105.8, 105.7, 84.5, 79.1, 78.6, 76.6, 76.29, 76.26, 76.2, 75.4, 74.9, 74.59, 74.55, 73.4, 73.13, 73.10, 68.5, 68.4, 66.8, 66.4, 65.9, 65.8, 51.8, 51.7, 49.8, 43.9, 43.4, 43.1, 42.4, 42.1, 40.5, 40.4, 39.3, 38.9, 38.7, 36.7, 33.8, 33.3, 30.5, 30.3, 30.2, 30.0, 29.9, 28.8, 27.9, 22.54, 22.48 ppm; HR-MS (ESI-TOF) m/z: [M+Na]⁺ Calcd. for C₃₀H₄₅IO₁₀Na⁺ 715.1950; Found 715.1946.

Alternative procedure for the synthesis of alcohol 21 from 20/C12-epi-20: Methyl [(1S,2S,4R,7S,9R,10S,12S,14R,16R)-12-(2-{(2S,5S)-5-[(3R,5R)-3-hydroxy-6-iodo-5-methylhept-6-en-1-yl]-4-methylidenetetrahydrofuran-2-yl}ethyl)-3,8,11,15,17-penta-oxapentacyclo[10.4.1.0^{2,7}.0^{9,16}.0^{10,14}]heptadec-4-yl]acetate (21): To a stirred solution of 20/C12-epi-20 (mixture of isomers, 2.2:1 dr, 55.7 mg, 0.0804 mmol, 1.0 equiv) in CH₂Cl₂/MeOH/H₂O (5.0 mL,

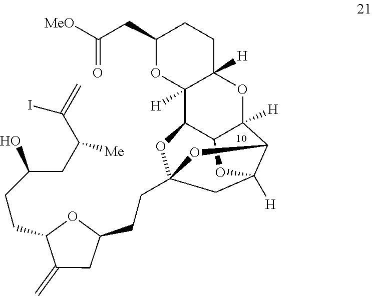

20:20:1, v/v/v) at 23° C. was added p-TsOH·H₂O (38.2 mg, 0.201 mmol, 2.5 equiv). The resulting mixture was stirred for 0.5 h before being diluted with NaHCO₃ solution (5 mL, sat. aq.). The layers were separated, and the aqueous layer was extracted with CH₂Cl₂ (3×15 mL). The combined organic extracts were dried over Na₂SO₄ and concentrated under reduced pressure. Flash column chromatography (SiO₂, hexanes/EtOAc 1:1, v/v→1:10, v/v, then EtOAc/MeOH 10:1, v/v) of the residue afforded alcohol 21 (32.1 mg, 0.0475 mmol, 86% yield from 20), and recovered C12-epi-20 (contaminated with trace amount of unreacted 20) (16.5 mg, 0.0239 mmol, 95% recovered from C12-epi-20) as colorless oils.
Methyl (2R)-2-{[(3R)-5-{[tert-butyl(diphenyl)silyl]oxy}pent-1-yn-3-yl]oxy}pent-4-enoate and methyl (2R)-2-{[(3S)-5-{[tert-butyl(diphenyl)silyl]oxy}pent-1-yn-3-yl]oxy}pent-4-enoate (24a and 24b): To a stirred solution of alkyne 23 (3.00 g, 8.86 mmol, 1.0 equiv) in CH₂Cl₂ (8 mL) at 23° C. was

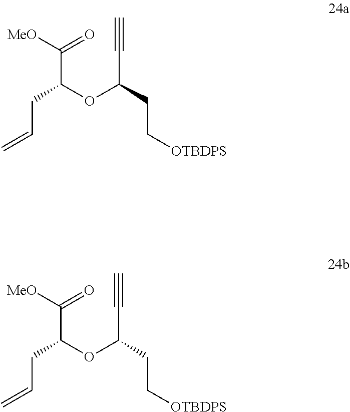

added Co₂(CO)₈ (3.64 g, 10.6 mmol, 1.2 equiv). After 20 min, a solution of olefin 22 (Mikami et al, 1986) (2.31 g, 17.7 mmol, 2.0 equiv) in CH₂Cl₂ (5 mL) was added. The resulting mixture was cooled to 0° C., and BF₃·Et₂O (2.19 mL, 17.7 mmol, 2.0 equiv) was added dropwise. After 0.5h, the reaction mixture was carefully quenched by addition of NaHCO₃ solution (50 mL, sat. aq.), and allowed to warm to 23° C. The aqueous layer was extracted with CH₂Cl₂(3×30 mL) and the combined organic extracts were dried over Na₂SO₄ and concentrated under reduced pressure.

To a stirred solution of the above obtained cobalt complex in acetone (60 mL) at 0° C. was added ceric ammonium nitrate (24.3 g, 44.3 mmol, 5.0 equiv) in three potions in 10-minute intervals and the resulting mixture was warmed to 23° C. and stirred for 1 h. The resulting mixture was concentrated under reduced pressure and the obtained residue was diluted with H₂O (50 mL) and extracted with EtOAc (3×60 mL). The combined organic extracts were washed with 1120 (50 mL), brine (30 mL), dried over Na₂SO₄, and concentrated under reduced pressure. Flash column chromatography (SiO₂, hexanes/EtOAc 20:1, v/v→5:1, v/v) of the residue afforded trans-isomer 24a (1.24 g, 2.75 mmol, 31% yield overall) and cis-isomer 24b (1.84 g, 4.08 mmol, 46% yield overall) as colorless oils, respectively. 24a: R_{f}=0.60 (SiO₂, hexanes/EtOAc 4:1, v/v);

$${[ α ]}_{D}^{23} = + 41. ( c = 2.2 , {CH}_{2} {Cl}_{2} ) ;$$

FT-IR (film): vₘₐₓ 3285, 2999, 2956, 2931, 1752, 1428, 1202, 1104, 919, 702 cm⁻¹; ¹H NMR (600 MHz, CDCl₃) δ 7.69-7.66 (m, 4H), 7.43-7.36 (m, 61H), 5.81 (ddt, J=17.1, 10.2, 6.9 Hz, 1H), 5.11 (dd, J=17.2, 1.5 Hz, 1H), 5.07 (dd, J=10.2, 1.5 Hz, 1H), 4.50 (tt, J=6.7, 1.7 Hz, 1H), 4.38 (ddd, J=6.9, 5.3, 1.3 Hz, 1H), 3.91-3.80 (m, 2H), 3.70 (s, 3H), 2.57-2.46 (m, 2H), 2.41 (d, J=2.1 Hz, 1H), 2.17-2.08 (m, 1H), 1.99-1.89 (m, 1H), 1.05 (d, J=1.4 Hz, 9H)ppm; ¹³C NMR (151 MHz, CDCl₃) δ 172.5, 135.8, 135.7, 133.9, 133.2, 129.7, 127.8, 127.7, 117.9, 82.1, 76.0, 74.8, 66.2, 60.0, 51.9, 38.9, 37.5, 26.9, 19.4 ppm; HRMS (ESI-TOF) m/z: [M+Na]⁺ Calcd. for C₂₇H₃₄O₄SiNa⁺ 473.2119; Found 473.2124, 24b: R_{f}=0.50 (SiO₂, hexanes/EtOAc 4:1, v/v);

$${[ α ]}_{D}^{23} = - 7.1 ( c = 1.5 , {CH}_{2} {Cl}_{2} ) ;$$

FT-IR (film): vₘₐₓ 3287, 2956, 2857, 1755, 1428, 1198, 1106, 822, 737, 701 cm⁻¹; ¹H NMR (600 MHz, CDCl₃) δ 7.66 (tt, J=6.2, 1.5 Hz, 41H), 7.45-7.35 (m, 6H), 5.77 (ddt, J=17.2, 10.2, 7.0 Hz, 1H), 5.11 (dd, J=17.2, 1.6 Hz, 1H), 5.06 (dd, J=10.2, 1.6 Hz, 1H), 4.52 (td, J=7.9, 6.3 Hz, 1H), 4.13 (t, J=6.3 Hz, 1H), 3.86 (ddd, J=10.4, 7.2, 4.8 Hz. 1H), 3.79 (ddd, J=10.4, 63, 5.1 Hz, 1H), 3.74 (s, 3H), 2.53-2.47 (m, 2H), 2.43 (d, J=2.1 Hz, 1H), 2.09 (dddd, J=13.8, 7.5, 6.2, 4.7 Hz, 1H), 1.98-1.89 (m, 1H), 1.06 (s, 9H)ppm; ¹³C NMR (151 MHz, CDCl₃) δ 172.6, 135.68, 135.66, 133.8, 133.7, 132.7, 129.8, 127.81, 127.79, 118.4, 82.2, 78.0, 74.5, 67.1, 59.7, 51.9, 38.9, 37.2, 26.9, 19.4 ppm; HRMS (ESI-TOF) m/z: [M+Na]⁺ Calcd. for C₂₇H₃₄O₄SiNa⁺ 473.2119; Found 473.2121.
Methyl (2R)-2-{[(3S)-5-{[tert-butyl(diphenyl)silyl]oxy}pent-1-en-3-yl]oxy}pent-4-enoate (S13): To

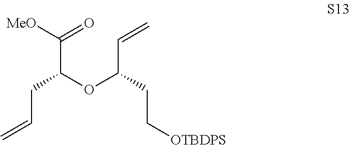

a stirred solution of alkyne 24b (1.50 g, 3.33 mmol, 1.0 equiv) in EtOAc (30 mL) at 23° C. were added quinoline (435 μL, 3.66 mmol, 1.1 equiv) and Lindlar catalyst (150 mg, 10% w/w). The reaction mixture was stirred under hydrogen atmosphere (1 bar) for 0.5 h before it was filtered through a pad of silica gel and washed with EtOAc (3×10 mL). The eluent was concentrated under reduced pressure. Flash column chromatography (SiO₂, hexanes/EtOAc 20:1, v/v→2:1, v/v) of the residue afforded bisalkene S13 (1.43 g, 3.16 mmol, 95% yield) as a colorless oil. S13: R_{f}=0.40 (SiO₂, hexanes/EtOAc 4:1, v/v);

$${[ α ]}_{D}^{23} = + 14.3 ( c = 2. , {CH}_{2} {Cl}_{2} ) ;$$

FT-IR (film): vₘₐₓ 3014, 2931, 2857, 1755, 1472, 1428, 1195, 1104, 1085, 921, 687 cm⁻¹; ¹H NMR (CDCl₃, 600 MHz) δ 7.68-7.63 (m, 4H), 7.45-7.35 (m, 6H), 5.82-5.68 (m, 2H), 5.22-5.00 (m, 4H), 4.03 (td, J=7.9, 5.6 Hz, 1H), 3.92 (t, J=6.3 Hz, 1H), 3.82 (ddt, J=10.7, 5.5, 3.2 Hz, 1H), 3.711-3.68 (m, 4H), 2.49-2.40 (m, 2H), 1.93 (ddt, J=13.4, 7.7, 5.61 Hz, 1H), 1.71 (ddt, J=13.4, 7.7, 5.61 Hz, 1H), 1.05 (s, 9H)ppm; ¹³C NMR (151 MHz, CDCl₃) δ 173.3, 138.6, 135.7, 134.03, 134.00, 133.2, 129.7, 127.8, 127.7, 118.2, 117.4, 79.9, 77.7, 60.1, 51.7, 38.4, 37.8, 27.0, 19.4 ppm; HRMS (ESI-TOF) m/z: [M+Na]⁺ Calcd. for C₂₇H₃₆O₄SiNa⁺ 475.2275; Found 475.2279.
(2R)-2-{1[(3S)-5-{[tert-Butyl(diphenyl)silyl]oxy}pent-1-en-3-yl]oxy}pent-4-enal (25): To a stirred

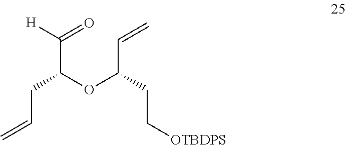

solution of ester S13 (1.20 g, 2.65 mmol, 1.0 equiv) in CH₂Cl₂ (25 mL) at −78° C. was added DIBAL-H (1.0M in toluene, 3.18 mL, 3.18 mmol, 1.2 equiv). The reaction mixture was stirred for 0.5 h before it was diluted with EtOAc (20 mL) and Rochelle salt solution (30 mL, sat. aq.). The mixture was warned to 23° C. and stirred for 2 h. The layers were separated, and the aqueous layer was extracted with EtOAc (3×30 mL). The combined organic extracts were washed with brine (20 mL), dried over Na₂SO₄, and concentrated under reduced pressure. Flash column chromatography (SiO₂, hexanes/EtOAc 20:1, v/v→5:1, v/v) of the residue afforded aldehyde 25 (1.04 g, 2.46 mmol, 93% yield) as a colorless oil. 25: R_{f}:0.50 (SiO₂, hexanes/EtOAc 4:1, v/v);

$${[ α ]}_{D}^{23} = + 24. ( c = 0.3 , {CH}_{2} {Cl}_{2} ) ;$$

FT-IR (film): vₘₐₓ 3072, 2952, 2931, 2858, 1734, 1472, 1428, 1389, 1111, 1089, 997, 926, 823, 738, 702 cm⁻¹; ¹H NMR (600 MHz, CDCl₃) δ 9.59 (d, J=1.8 Hz, 1H), 7.69-7.62 (m, 4H), 7.45-7.40 (m, 2H), 7.41-7.34 (m, 4H), 5.82-5.63 (m, 2H), 5.20 (dt, J=1.5, 0.9 Hz, 1H), 5.18 (ddd, J=4.8, 1.5, 0.7 Hz, 1H), 5.09 (dd, J=17.1, 1.7 Hz, 1H), 5.06-5.01 (m, 1H), 4.08 (td, J=7.9, 5.4 Hz, 1H), 3.82 (ddd, J=10.4, 7.3, 5.3 Hz, 1H), 3.78 (ddd, J=7.2, 5.5, 1.9 Hz, 1H), 3.71 (dt, J=10.4, 5.81 Hz, 1H), 2.45-2.32 (m, 2H), 1.93 (dddd, J=13.8, 7.7, 6.0, 5.3 Hz, 1H), 1.74 (ddt, J=13.9, 7.4, 5.6 Hz, 1H), 1.05 (s, 9H)ppm; ¹³C NMR (151 MHz, CDCl₃) δ 204.2, 138.5, 135.7, 1330.90, 1330.89, 132.8, 129.8, 127.79, 127.78, 118.8, 118.5, 82.3, 79.4, 60.1, 38.6, 35.0, 27.0, 19.4 ppm; HRMS (ESI-TOF) m/z: [M+Na]⁺ Calcd. for C₂₆H₃₄O₃SiNa⁺ 445.2169; Found 445.2170.
(3aS,4S,6R)-4-(2-{[tert-Butyl(diphenyl)silyl]oxy}ethyl)-6-(prop-2-en-1-yl)-3a,4-dihydro-3H,61!-furo[3,4-c][1,2]oxazole (26): To a stirred solution of aldehyde 25 (1.04 g, 2.46 mmol, 1.0 equiv) in

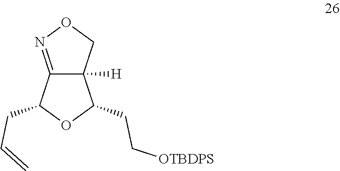

pyridine/EtOH (15 mL, 5:1, v/v) at 23° C. was added hydroxylammonium chloride (375 mg, 5.40 mmol, 2.2 equiv). The reaction mixture was stirred for 0.5 h before it was quenched by addition of NaHCO₃ solution (10 mL, sat. aq.). The layers were separated, and the aqueous layer was extracted with EtOAc (3×20 mL). The combined organic extracts were washed with brine (20 mL), dried over Na₂SO₄, and concentrated under reduced pressure to obtain the crude corresponding hydroxylamine.

To a stirred solution of the so-obtained crude hydroxylamine in CH₂Cl₂ (15 mL) at 23° C. was added NaOCl solution (1.31 mL, 21.2 mmol, 8.0 equiv, available chlorine 10-15%). The reaction mixture was stirred for 3 h before it was diluted with water (30 mL). The layers were separated, and the aqueous layer was extracted with EtOAc (3×20 mL). The combined organic extracts were washed with brine (1.0 mL), dried over Na₂SO₄, and concentrated under reduced pressure. Flash column chromatography (SiO₂, hexanes/EtOAc 20:1, v/v→5:1, v/v) of the residue afforded isoxazoline 26 (716 mg, 1.64 mmol, 62% yield overall) as a white foam. 26: R_{f}=0.60 (SiO₂, hexanes/EtOAc 9:1, v/v);

$${[ α ]}_{D}^{23} = + 51.3 ( c = 0.8 , {CH}_{2} {Cl}_{2} ) ;$$

FT-IR (film): vₘₐₓ 2999, 2931, 2858, 1472, 1428, 1111, 1088, 998, 822, 702 cm⁻¹; ¹H NMR (600 MHz, CDCl₃) δ 7.65-7.61 (m, 4H), 7.46-7.42 (m, 2H), 7.41-7.37 (m, 4H), 5.81 (ddt, J=17.2, 10.2, 7.0 Hz, 1H), 5.20-5.10 (m, 2H), 4.62 (td, J=6.0, 1.6 Hz, 1H), 4.43 (dd, J=9.6, 8.4 Hz, 1H), 4.03 (dt, J=9.5, 6.4 Hz, 1H), 3.97 (dd, J=12.4, 8.4 Hz, 1H), 3.82-3.73 (m, 2H), 3.70 (ddd, J=10.7, 7.1, 4.8 Hz, 1H), 2.52-2.41 (m, 2H), 2.06-1.95 (m, 1H), 1.75 (dtd, J=13.6, 6.7, 4,8-fz, 1H), 1.05 (s, 9H)ppm; ¹³C NMR (151 MHz, CDCl₃) δ 171.6, 135.6, 133.5, 133.4, 132.4, 130.0, 129.9, 127.9, 127.8, 119.0, 79.9, 72.8, 72.2, 60.5, 60.4, 38.3, 37.4, 27.0, 19.3 ppm; HRMS (ESI-TOF) m/z: [M+Na]⁺ Calcd. for C₂₆H₃₃ NO₃SiNa⁺ 458.2122; Found 458.2131.

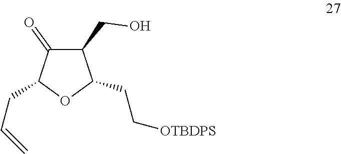

(1R)-1,4-Anhydro-6-O-[tert-butyl(diphenyl)silyl]-3,5-dideoxy-3-(hydroxymethyl)-1-prop-2-en-1-yl-L-erythro-hex-2-ulose (27): To a stirred OTBDPs solution of isoxazoline 26 (800 mg, 1.84 mmol, 1.0 equiv) in MeCN/H₂O (15 mL, 4:1, v/v) at 23° C. was added Mo(CO)₆ (969 mg, 3.67 mmol, 2.0 equiv). The reaction mixture was heated to 80° C. and stirred at this temperature for 2 h. The reaction mixture was then allowed to cool to 23° C. and diluted with water (10 mL). The layers were separated, and the aqueous layer was extracted with EtOAc (3×20 mL). The combined organic layers were washed with brine (10 mL), dried over Na₂SO₄, and concentrated under reduced pressure. Flash column chromatography (SiO₂, hexanes/EtOAc 10:1, v/v→5:1, v/v) of the residue afforded keto alcohol 27 (580 mg, 1.32 mmol, 72% yield) as a white foam. 27: R_{f}=0.30 (SiO₂, hexanes/EtOAc 4:1, v/v);

$${[ α ]}_{D}^{23} = + 27.6 ( c = 0.7 , {CH}_{2} {Cl}_{2} ) ;$$

FT-IR (film): vₘₐₓ 3482, 3071, 2930, 2857, 1757, 1472, 1428, 1111, 822, 702 cm⁻¹; ¹H NMR (CDCl₃, 600 MHz) δ 7.68-7.66 (m, 41H), 7.46-741 (m, 2H), 7.39 (dd, J=7.9, 6.6 Hz, 4H), 5.78 (ddt, J=17.1, 10.2, 6.91 Hz, 1H), 5.16-5.04 (m, 2H), 421 (ddd, J=10.3, 7.0, 4.6 Hz, 1H), 3.92-3.88 (m, 3H), 3.84-3.78 (m, 2H), 2.52 (dddt, J=13.8, 6.0, 4.5, 1.3 Hz, 1H), 2.36-2.29 (m, 2H), 2.15 (dd, J=7.0, 5.0 Hz, 1H), 2.07-1.93 (m, 2H), 1.05 (s, 9H)ppm; ¹³C NMR (151 MHz, CDCl₃) δ 217.7, 135.71, 135.69, 133.7, 133.6, 133.0, 129.9, 129.8, 127.9, 127.8, 118.3, 80.9, 75.2, 60.2, 59.5, 55.0, 37.4, 35.3, 27.0, 19.3 ppm; HRMS (ESI-TOF) ma z: [M+Na]⁺ Calcd. for C₂₆H₃₄O₄SiNa⁺ 461.2129; Found 461.2125. (1R)-1,4-Anhydro-6-O-[tert-butyl(diphenyl)silyl]-3,5-dideoxy-3-(hydroxymethyl)-1-prop-2-en-1-yl-L-arabino-hexitol (28): To a stirred solution of keto alcohol 27 (2.00 g, 4.56 mmol, 1.0 equiv) in MeOH (40 mL) at 0° C. was added tetramethylammonium triacetoxyborohvdride (1.80 g, 6.84 mmol,

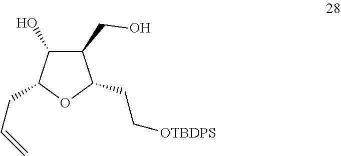

1.5 equiv). The reaction mixture was stirred for 0.5 h before it was concentrated under reduced pressure. The residue was diluted with water (30 mL) and extracted with EtOAc (3×30 mL). The combined organic extracts were dried over Na₂SO₄ and concentrated under reduced pressure. Flash column chromatography (SiO₂, hexanes/EtOAc 10:1, v/v→2:1, v/v) of the residue afforded diol 28 (1.91 g, 4.33 mmol, 95% yield) as a white foam. 28: R_{f}=0.30 (SiO₂, hexanes/EtOAc 1:1, v/v);

$${[ α ]}_{D}^{23} = - 25.5 ( c = 0.6 , {CH}_{2} {Cl}_{2} ) ;$$

FT-IR (film): vₘₐₓ 3379, 2930, 2857, 1472, 1427, 1110, 1087, 998, 823, 737, 701 cm⁻¹; ¹H NMR (CDCl₃, 600 MHz) δ7.69-7.63 (m, 4H), 7.45-7.41 (m, 2H), 7.40-7.35 (m, 4H), 5.87 (ddt, J=17.1, 10.2, 6.9 Hz, 1H), 5.16 (dd, J=17.2, 1.2 Hz, 1H), 5.09 (dd, J=10.2, 1.2 Hz, 1H), 4.07 (dd, J=4.0, 2.3 Hz, 1H), 3.90-3.80 (m, 2H), 3.76-3.72 (m. 1H), 3.68 (dd, J=6.9, 4.1 Hz, 3H), 2.43 (tt, J=7.0, 1.4 Hz, 2H), 2.13 (qd, J=6.9, 2.4 Hz, 1H), 1.94 (dtd, J=14.0, 6.5, 5.3 Hz, 1H), 1.85 (ddt, J=14.0, 7.2, 5.61 Hz, 1H), 1.05 (s, 9H)ppm; Two proton resonance signals could not be observed due to exchange of the proton with residual water in the solvent used; ¹³C NMR (151 MHz, CDCl₃) δ 135.8, 135.7, 134.9, 133.7, 133.6, 129.9, 129.8, 127.9, 127.8, 117.2, 81.0, 77.6, 76.4, 63.2, 61.3, 56.5, 38.3, 33.7, 27.0, 19.3 ppm; HRMS (ESI-TOF) m/z: [M+Na]⁺ Calcd. for C₂₆H₃₆O₄SiNa⁺ 463.2275; Found 463.2278.
(1R)-1,4-Anhydro-6-O-[tert-butyl(diphenyl)silyl]-3,5-dideoxy-3-{[(4-methylphenyl)sulfanyl]methyl}-1-prop-2-en-1-yl-L-arabino-hexitol (29): To a stirred solution of diol 28 (1.50 g, 3.40 mmol, 1.0 equiv) in pyridine (15 mL) at 23° C. were added p-tolyl disulfide (2.52 g,

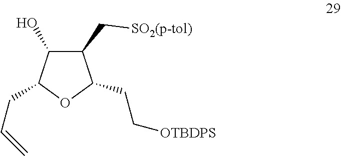

10.2 mmol, 3.0 equiv) and n-tributyl phosphine (2.55 mL. 10.2 mmol, 3.0 equiv). The reaction mixture was stirred for 3 h and was then concentrated under reduced pressure. Flash column chromatography (SiO₂, hexanes/EtOAc 10:1, v/v→2:1, v/v) of the residue afforded sulfide 29 (1.68 g, 3.06 mmol, 90% yield) as a white foam. 29: R_{f}=0.20 (SiO₂, hexanes/EtOAc 4:1, v/v);

$${[ α ]}_{D}^{23} = - 16.6 ( c = 0.5 , {CH}_{2} {Cl}_{2} ) ;$$

FT-IR (film): vₘₐₓ 3449, 3071, 2956, 2929, 2857, 1492, 1427, 1261, 1110, 1091, 803, 702 cm⁻¹; ¹H NMR (600 MHz, CDCl₃) δ 7.58 (dd, 7.6, 1.4 Hz, 4H), 7.36-7.31 (m, 2H), 7.29 (t, J=7.6 Hz. 4H), 7.16 (d, J=8.0 Hz, 2H), 6.99 (d, J=8.0 Hz, 2H), 5.78 (ddt, J=17.1, 10.3, 7.0 Hz, 1H), 5.07 (dd, J=17.3, 2.1 Hz, 1H), 4.99 (dd, J=10.1, 2.1 Hz, 1H), 4.00-3.94 (m, 1H), 3.71 (dddd, J=23.8, 11.6, 8.5, 5.4 Hz, 3H), 3.63 (td, J=7.0, 3.8 Hz, 1H), 2.88-2.80 (m, 2H), 2.35 (t, J=7.0 Hz, 2H), 2.22 (s, 3H), 2.01 (dddd, J=24.0, 9.9, 6.5, 2.71 Hz, 2H), 1.81-1.71 (m, 2H), 0.96 (s, 9H)ppm; ¹³C NMR (151 MHz, CDCl₃) δ 136.7, 135.7, 135.6, 134.9, 133.8, 133.7, 1319, 130.4, 129.9, 129.8, 129.7, 127.8, 127.7, 117.2, 81.0, 79.9, 78.1, 61.0, 53.2, 38.0, 36.7, 33.6, 26.9, 21.2, 193 ppm; HRMS (ESI-TOF) m/z: [M+Na]⁺ Calcd. for C₃₃H₄₂O₃SSiNa⁺ 569.2516; Found 569.2520.
(1R)-1,4-Anhydro-6-O-[tert-butyl(diphenyl)silyl]-3,5-dideoxy-2-O-methyl-3-{[(4-methylphenyl)-sulfonyl]methyl}-1-prop-2-en-1-yl-L-arabino-hexitol (30): To a stirred solution of sulfide 29 (1.00 g, 1.83 mmol 1.0 equiv) in TIF/DMF (15 mL, 1:1, v/v) at 0° C. were added sodium hydride (60%

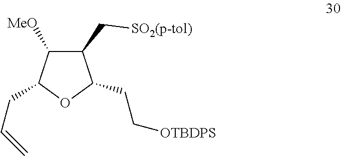

dispersion in mineral oil, 293 mg, 7.32 mmol, 4.0 equiv) and methyl iodide (0.683 mL, 1.0 mmol, 6.0 equiv). The reaction mixture was allowed to warn to 23° stirred at this temperature for 0.5 h before it was quenched by addition of NH₄Cl solution (10 mL, sat. aq.). The layers were separated, and the aqueous layer was extracted with EtOAc (3×20 mL). The combined organic extracts were washed with brine (10 mL), dried over Na₂SO₄, and concentrated under reduced pressure.

To a stirred solution of the so-obtained crude residue in THF/H₂O (12 mL, 1:1, v/v) at 23° C. was added oxone (835 mg, 5.49 mmol, 3.0 equiv). The resulting mixture was stirred for 2 h before it was diluted with H₂O (10 mL). The layers were separated, and the aqueous layer was extracted with EtOAc (3×20 mL). The combined organic extracts were washed with brine (10 mL), dried over Na₂SO₄, and concentrated under reduced pressure. Flash column chromatography (SiO₂, hexanes/EtOAc 10:1, v/v→3:1, v/v) of the residue afforded sulfoxide 30 (976 mg, 1.65 mmol, 90% yield overall) as a white foam. 30: R_{f}=0.30 (SiO₂, hexanes/EtOAc 4:1, v/v);

$${[ α ]}_{D}^{23} = - 28.2 ( c = 2.5 , {CH}_{2} {Cl}_{2} ) ;$$

FT-IR (film): vₘₐₓ 3071, 2930, 2856, 1597, 1472, 1319, 1146,1105, 1085, 998,821, 701 cm⁻¹; ¹HNMR (600 MHz, CDCl₃) δ 7.78 (d, J=8.0 Hz, 2H), 7.64 (ddt, J=7.8, 4.0, 1.5 Hz, 4H), 7.45-7.34 (m, 6H), 7.32 (d, J=8.0 Hz, 2H), 5.83 (dddd, J=18.4, 10.2, 7.6, 6.4 Hz, 1H), 5.14 (dd, J=17.2, 1.2 Hz, 1H), 5.07 (dd, J=10.2, 1.2 Hz, 1H), 3.89 (d, J=3.3 Hz, 1H), 3.78 (dt, J=11.9, 6.2 Hz, 1H), 3.73-3.64 (m, 3H), 3.42 (s, 3H), 3.16-3.05 (m, 2H), 2.52-2.40 (m, 6H), 1.95 (dq, 3=13.5, 6.5 Hz, 1H), 1.79-1.69 (m, 1H), 1.00 (s, 9H)ppm; ¹³C NMR (151 MHz, CDCl₃) δ 145.0, 136.7, 135.7, 135.6, 135.0, 133.9, 133.7, 130.1, 129.8, 129.7, 128.0, 127.8, 127.7, 117.1, 85.5, 81.4, 80.6, 60.9, 58.1, 57.7, 44.2, 38.1, 33.3, 27.0, 21.8, 19.3 ppm; HRMS (ESI-TOF) m/z: [M+Na]⁺ Calcd. for C₃₄H₄₄O₅SSiNa⁺ 615.2571; Found 615.2576.
(1R)-1,4-Anhydro-3,5-dideoxy-6-O-(2,2-dimetbylpropanoyl)-2-O-methyl-3-{[(4-methylphenyl)-

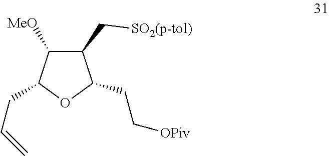

sulfonyl]methyl}-1-prop-2-en-1-yl-L-arabino-hexitol (31): To a stirred solution of sulfoxide 30 (1.50 g, 2.53 mmol, 1.0 equiv) in THF (12 mL) at 0° C. was added HF·py (70% HF, 6.58 mL, 253 mmol, 100 equiv). The reaction mixture was warmed to 23° C. and stirred at this temperature for 2 h before it was quenched by addition of NaHCO₃ solution (80 mL, sat. aq.). The layers were separated, the aqueous layer was extracted with EtOAc (3×20 mL), and the combined organic layers were dried over Na₂SO₄ and concentrated under reduced pressure.

To a stirred solution of the so-obtained crude residue in pyridine/CH₂Cl₂ (10 mL, 1:1, v/v) at −20° C. was added pivaloyl chloride (467 μL, 3.80 mmol, 1.5 equiv). The reaction mixture was stirred for 1.5 h before it was quenched by addition of NaHCO₃ solution (20 mL, sat. aq.). The layers were separated, the aqueous layer was extracted with EtOAc (3×20 mL), and the combined organic layers were dried over Na₂SO₄ and concentrated under reduced pressure. Flash column chromatography (SiO₂, hexanes/EtOAc 20:1, v/v→4:1, v/v) of the residue afforded olefin 31 (1.04 g, 2.38 mmol, 94% yield overall) as a white foam. 31: R_{f}=0.20 (SiO₂, hexanes/EtOAc 4:1, v/v);

$${[ α ]}_{D}^{23} = - 34.4 ( c = 1.8 , {CH}_{2} {Cl}_{2} ) ;$$

FT-IR (film): vₘₐₓ 2973, 2873, 1722, 1480, 1399, 1302, 1286, 1148, 1087, 915, 759 cm¹; ¹H NMR (600 MHz, CDCl₃) δ 7.80 (d, J=8.0 Hz, 2H), 7.38 (d, J=8.0 Hz, 2H), 5.80 (ddt, J=17.2, 10.2, 7.0 Hz, 1H), 5.12 (dd, J=17.2, 1.9 Hz, 1H), 5.05 (dd, J=10.1, 1.9 Hz, 1H), 4.09 (t, J=6.6 Hz, 2H), 3.79 (dd, J=3.7, 1.1 Hz, 1H), 3.71 (td, J=6.9, 3.5 Hz, 1H), 3.62 (dt, J=8.4, 4.7 Hz, 1H), 3.39 (s, 3H), 3.09 (dd, J=14.2,8.9 Hz, 1H), 3.03 (dd, J=14.2, 5.1 Hz, 1H), 2.54-2.48 (m, 1H), 2.46 (s, 3H), 2.45-2.42 (m, 2H), 1.99-1.86 (m, 2H), 1.17 (s, 9H)ppm; ¹³C NMR (151 MHz, CDCl₃) δ 178.5, 145.3, 136.6, 134.8, 130.2, 128.0, 117.3, 85.7, 81.4, 80.8, 61.5, 58.4, 57.6, 44.0, 38.8, 34.8, 33.4, 27.3, 21.8 ppm; HRMS (ESI-TOF) m/z: [M+Na]⁺ Calcd. for C₂₃H₃₄O₆SNa⁺ 461.1968; Found 461.1977.
3,6-Anhydro-8,9-bis-O-[tert-butyl(dimethyl)silyl]-2,4,7-trideoxy-1-O-(2,2-dimnethylpropanoyl)-5-O-methyl-4-{[(4-methylphenyl)sulfonyl]methyl}-D-glycero-D-gulo-nonitol and 3,6-anhydro-8,9-bis-O-[tert-butyl(dimethyl)silyl]-2,4,7-trideoxy-1-O-(2,2-dimethylpropanoyl)-5-O-methyl-4-{[(4-methylphenyl)sulfonyl]methyl}-L-glycero-D-gulo-nonitol (32 and C2-epi-32): To a stirred solution of olefin 31 (900 mg, 2.05 mmol, 1.0 equiv) in t-BuOH/H₂O (40 mL, 1:1, v/v) at −5° C. were added AD-mix-α

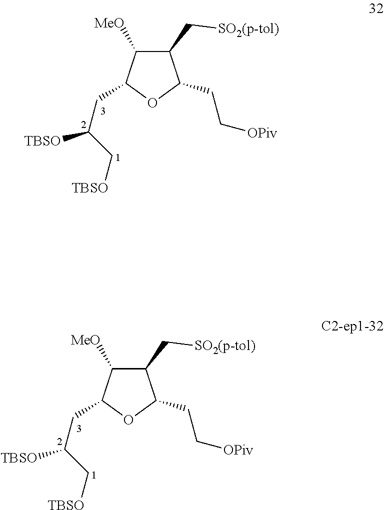

(3.28 g, 1.60 g/mmol of 31) and methane sulfonamide (205 mg, 100 mg/mmol of 31). The reaction mixture was stirred at −5° C. for 36 h before it was allowed to warm to 23° C. and diluted with water (30 mL). The layers were separated, the aqueous layer was extracted with EtOAc (3×30 mL) and the combined organic layers were dried over Na₂SO₄ and concentrated under reduced pressure.

To a stirred solution of the so-obtained crude diol in CH₂Cl₂(10 mL) at 0° C. were added imidazole (670 mg, 9.85 mmol, 4.8 equiv), DMAP (75.0 mg, 0.616 mmol, 0.3 equiv), and TBSCl (928 mg, 6.16 mmol, 3.0 equiv). The reaction mixture was warmed to 23° C. and further stirred for 48 h before it was quenched by addition of NaHCO₃ solution (10 mL, sat. aq.). The layers were separated, and the aqueous layer was extracted with CH₂Cl₂ (3×30 mL). The combined organic layers were dried over Na₂SO, and concentrated under reduced pressure. Flash column chromatography (SiO₂, hexanes/EtOAc 20:1, v/v→4:1, v/v) of the residue afforded bis-silyl ethers 32 (1.00 g, 1.44 mmol, 70% yield overall) and C2-epi-32 (201 mg, 0.287 mmol, 14% yield overall) as white foams, respectively. 32: R_{f}=0.80 (SiO₂, hexanes/EtOAc 3:2, v/v);

$${[ α ]}_{D}^{23} = - 2 9.9 ( c = 1.8 , {CH}_{2} {Cl}_{2} ) ;$$

FT-IR (film): vₘₐₓ 2955, 2929, 2857, 1726, 1598, 1472, 1320, 1252, 1150, 1087, 833, 775 cm⁻¹; (600 MHz, CDCl₃) δ 7.80 (d, J=8.0 Hz, 2H), 7.38 (d, J=8.0 Hz, 2H), 4.08 (td, J=6.6, 3.6 Hz, 2H), 3.87 (td, J=6.6, 3.6 Hz, 1H), 3.82-3.74 (m, 2H), 3.61-3.50 (m, 2H), 3.47 (dd, J=10.3, 5.2 Hz, 1H), 3.38 (s, 3H), 3.10-2.97 (m, 2H), 2.52-2.42 (m, 4H), 2.00-1.86 (m, 3H), 1.78 (dt, J=13.8, 6.8 Hz, 1H), 1.17 (s, 9H), 0.88 (s, 9H), 0.87 (s, 9H), 0.07 (s, 3H), 0.07 (s, 3H), 0.04 (s, 3H), 0.03 (s, 3H)ppm; ¹³C NMR (151 MHz, CDCl₃) δ 178.5, 145.2, 136.6, 130.2, 128.0, 85.9, 80.4, 78.6, 71.4, 67.9, 61.6, 58.4, 57.6, 44.1, 38.8, 34.7, 33.6, 27.3, 26.2, 26.1, 21.8, 18.5, 18.3, −3.9, −4.6, −5.2, −5.3 ppm; HRMS (ESI-TOF) m/z: [M+Na]⁺ Calcd. for C₃₅H₆₄O₈SSiO₂Na⁺ 723.3753; Found 723.3751. C2-epi-32: R_{f}=0.78 (SiO₂, hexanes/EtOAc 3:2, v/v);

$${[ α ]}_{D}^{23} = - 10. ( c = 0.80 , {CH}_{2} {Cl}_{2} ) ;$$

FT-IR (film): vₘₐₓ 2956, 2929, 2857, 1727, 1462, 1286, 1252, 1150, 1102, 1005, 834, 776 cm⁻¹; ¹H NMR (600 MHz, CDCl₃) δ 781 (d, J=8.0 Hz, 2H), 7.39 (d, J=8.0 Hz, 2H), 4.15-4.04 (m, 2H), 3.84 (ddt, J=16.7, 9.9, 3.3 Hz, 2H), 3.73 (dd, 0.1=3.7, 1.5 Hz, 1H), 3.53 (dd, J=10.1, 5.4 Hz, 1H), 3.47 (ddt, J=12.0, 10.1, 4.9 Hz, 2H), 3.37 (s, 3H), 3.09 (dd, J=14.2, 9.1 Hz, 1H), 3.01 (dd, J=14.2, 4.9 Hz, 1H), 2.46 (s, 3H), 2.39 (dddd, J=9.i, 6.2, 4.9, 1.5 Hz, 1H), 1.97-1.83 (m, 2H), 1.63-1.53 (m, 2H), 1.17 (s, 9H), 0.88 (s, 9H), 0.86 (s, 9H), 0.06 (s, 3H), 0.05 (s, 3H), 0.04 (s, 3H), 0.01 (s, 3H)ppm; ¹³C NMR (151 MHz, CDCl₃) δ 178.5, 145.2, 136.6, 130.2, 128.1, 86.9, 80.0, 77.9, 70.7, 68.6, 61.6, 58.3, 57.9, 45.1, 38.8, 34.5, 33.6, 27.3, 26.2, 26.1, 21.8, 18.6, 18.3, −4.1, −4.8,-5.2, −5.1 ppm; HRMS (ESI-TOF) m/z: [M+Na]⁺ Calcd. for C₃₅H₆₄O₈SSiO₂Na⁺ 723.3753; Found 723.3756.
3,6-Anhydro-8,9-bis-O-[tert-butyl(dimethyl)silyl]-2,4,7-trideoxy-5-O-

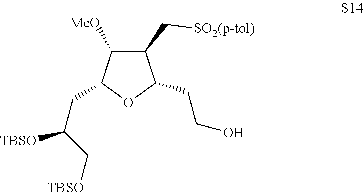

methyl-4-{[(4-methylphenyl)sulfonyl]methyl}-D-glycero-D-gulo-nonitol (S14): To a stirred solution of pivaloyl ester 32 (500 mg, 0.713 mmol, 1.0 equiv) in MeOH (5 mL) at 0° C. was add NaOMe (0.5M in MeOH, 7.13 mL, 3.57 mmol, 5.0 equiv). The reaction mixture was allowed to warm to 23° C. and stirred for 2 h. The reaction mixture was cooled to 0° C. and quenched by addition of NH₄Cl solution (15 mL, sat. aq.). The aqueous layer was extracted with EtOAc (3×20 mL) and the combined organic layers were dried over Na₂SO₄ and concentrated under reduced pressure. Flash column chromatography (SiO₂, hexanes/EtOAc 5:1, v/v→1:1, v/v) of the residue afforded alcohol S14 (387 mg, 0.627 mmol, 88% yield) as a colorless oil. S14: R_{f}=0.30 (SiO₂, hexanes/EtOAc 3:2, v/v)

$${[ α ]}_{D}^{23} = - 32. ( c - 2. , {CH}_{2} {Cl}_{2} ) ;$$

FT-IR (film): vₘₐₓ 3504, 2953, 2929, 2857, 1598, 1472, 1320, 1253, 1147, 1087, 834, 775 cm 1; ¹H NMR (600 MHz, CDCl₃) δ 7.79 (d, J=8.0 Hz, 2H), 7.38 (d, J=8.0 Hz, 2H), 3.87 (td, J=6.5, 3.5 Hz, 1H), 3.79-3.76 (m, 1H), 3.76-3.67 (m, 4H), 3.56 (dd, J=10.3, 5.5 Hz, 1H), 3.47 (dd, J=10.3, 5.4 Hz, 1H), 3.38 (s, 3H), 3.10-3.02 (m, 2H), 2.60-2.53 (m, 1H), 2.46 (s. 3H), 2.38 (brs, 1H), 1.97 (dt, J=14.0, 5.71 Hz, 1H), 1.93-1.75 (m, 3H). 0.88 (s, 9H), 0.87 (s, 9H), 0.08 (s, 3H), 0.07 (s, 3H), 0.04 (s, 3H), 0.03 (s, 3H)ppm; ¹³C NMR (151 MHz, CDCl₃) δ 145.2, 136.7, 130.2, 128.0, 86.2, 83.0, 78.8, 71.4, 67.9, 60.7, 58.5, 57.5, 44.0, 36.9, 33.6, 26.2, 26.L 21.8, 18.5, 18.3, −4.0, −4.6, −5.1, −5.2 ppm; HRMS (ESI-TOF) m/z: [M+Na]⁺ Calcd. for C₃₀H₅₆O₇SSiO₂Na⁺ 639.3177; Found 639.3180.
3,6-Anhydro-8,9-bis-O-[tert-butyl(dimethyl)silyl]-2,4,7-trideoxy-5-O-methyl-4-{[(4-methyl-

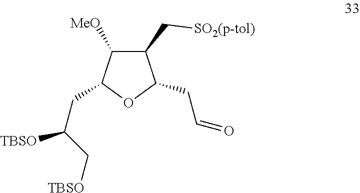

phenyl)sulfonyl]methyl}-D-glycero-D-gulo-nonose (33): To a stirred solution of alcohol S14 (300 mg, 0.486 mmol, 1.0 equiv) in dry CH₂Cl₂ (10 mL) at 0° C. was added Dess-Martin periodinane (516 mg, 1.22 mmol, 2.5 equiv). The resulting mixture was allowed to warn to 23° C. and stirred for 0.5 h before it was diluted with H₂O (10 mL) and Na₂S₂O₃ solution (15 mL, sat. aq.). The resulting mixture was vigorously stirred for 3 h. The layers were separated, and the aqueous layer was extracted with CH₂Cl₂ (3×30 mL). The combined organic extracts were washed with brine (30 mL), dried over Na₂SO₄, and concentrated under reduced pressure. Flash column chromatography (SiO₂, hexanes/EtOAc 5:1, v/v→1:1, v/v) of the residue afforded aldehyde 33 (275 mg, 0.447 mmol, 92% yield) as a white foam. 33: R_{f}=0.70 (SiO₂, hexanes/EtOAc 3:2, v/v);

$${[ α ]}_{D}^{23} = - 14.3 ( c = 2. , {CH}_{2} {Cl}_{2} ) ;$$

FT-IR (film): vₘₐₓ 2954, 2929, 2857, 1752, 1472, 1463, 1361, 1318, 1253, 1148, 1088, 1005, 835, 776 cm¹;¹H NMR (600 MHz, CDCl₃) δ 9.70 (t, J=1.4 Hz, 1H), 7.80 (d, J=8.2 Hz, 2H), 7.39 (d, J=8.2 Hz, 2H), 3.97 (td, J=6.2, 4.7 Hz, 1H), 3.93 (td, J=6.2, 3.55 Hz, 1H), 3.82 (dd, J=3.5, 1.1 Hz, 1H), 3.76 (dq, J=6.7, 5.5 Hz, 1H), 3.55 (dd, J=10.2, 5.51 Hz, 1H), 3.45 (dd, J=10.2, 5.5 Hz, 1H), 3.37 (s, 3H), 3.29 (dd, J=14.1, 5.1 Hz, 1H), 3.07 (dd, J=14.1, 9.0 Hz, 1H), 2.88 (ddd, J=17.5, 6.5, 1.81 Hz, 1H), 2.77 (ddd, J=17.5, 5.9, 1.2 Hz, 1H), 2.49 (ddd, J=9.0, 5.0, 121 Hz, 1H), 2.46 (s, 3H), 1.96 (ddd, J=13.9, 6.3, 5.11 Hz, 1H), 1.78 (dt, J=13.9, 6.8 Hz, 1H), 0.88 (s, 9H), 0.87 (s, 9H), 0.07 (s, 6H), 0.04 (s, 3H), 0.03 (s, 3H)ppm; ¹³C NMR (151 MHz, CDCl₃) δ 200.9, 145.2, 136.5, 130.2, 128.0, 85.9, 78.6, 78.3, 71.3, 67.9, 58.0, 57.5, 49.8, 44.3, 33.5, 26.2, 26.1, 218, 18.5, 18.3, −3.9, −4.6, −5.3, −5.2 ppm; HRMS (ESI-TOF) rnz: [M+Na]⁺ Calcd. for C₃₀H₅₄O₇SSiO₂Na⁺ 637.3021; Found 637.3016.
Methyl [(1S,2S,4R,7S,9R,10S,12S,14R,16R)-12-{2-1(2S,5S)-5-{2-1(2S,4R,6R)-6-{[(2S,3S,4R,5R)-5-[(2S)-2,3-bis{[tert-butyl(dimethyl)silyljoxy}propylj-4-methoxy-3-{[(4-methylphenyl)sulfonyl]-methyl}tetrahydrofuran-2-yl]methyl}-4-mnethyl-5-methiylidenetetrahydro-2H-pyran-2-yl]ethyl}-4-methylidenetetrahydrofuran-2-yl]ethyl}-3,8,11,15,17-pentaoxapentacyclo-

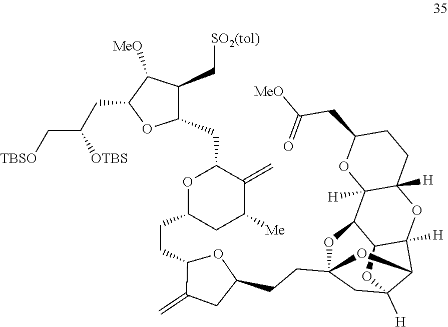

[10.4.1.0^{2,7}.0^{9,16}.0^{10,14}]heptadec-4-yl]acetate (35): In a glove box, to a stirred solution of 33 (46.9 mg, 0.102 mmol, 3.0 equiv) and 8 (25.6 mg. 0.0340 mmol, 1.0 equiv) in THF/DMF (1.0 mL, 4:1, v/v) at 23° C. were added a 2.0% NiCl₂/CrCl₂ (42.0 mg, 0.340 mmol, 10 equiv) mixture. The resulting mixture was vigorously stirred for 24 h before it was quenched by addition of H₂O (10 mL). The layers were separated, and the aqueous layer was extracted with EtOAc (4×10 mL). The combined organic extracts were washed with brine (10 mL), dried over Na₂SO₄, and concentrated under reduced pressure. Flash column chromatography (SiO₂, hexanes/EtOAc 3:1, v/v→1:3, v/v) of the residue afforded the corresponding all alcohol intermediate (17.4 mg, 0.0160 mmol, 47% yield) as a light green oil (Note: The ligand free method was used when the material was prepared at the early stages of our halichondrin project).

To a stirred solution of above obtained ally alcohol (17.4 mg, 0.0160 mmol, 1.0 equiv) in toluene (2 mL) at 23° C. were added DBU (200 μL, 6.70 mmol, 84 equiv, Note: toluene:DBU 10:1, v/v). The resulting mixture was heated to 110° C. and stirred for 1 h before it was allowed to cool to 23° C. and concentrated under reduced pressure. Flash column chromatography (SiO₂, hexanes/EtOAc 3:1, v/v→1:5, v/v) of the residue afforded ester 35 (12.8 mg, 0.0130 mmol, 81% yield) as a colorless oil. 35: R_{f}=0.50 (SiO₂, hexanes/EtOAc 1:1, v/v);

$${[ α ]}_{D}^{23} = - 30.4 ( c = 0.50 , EtOAc ) ;$$

FT-IR (film): vₘₐₓ 2952,2929, 2856, 1740, 1462, 1437, 1361, 1323, 1289, 1254, 1190, 1134, 1084, 1015, 835, 777 cm⁻¹; ¹H NMR (600 MHz, C₆D₆) δ 7.91 (d, J=8.3 Hz, 2H), 6.91 (d, J=7.9 Hz, 2H), 4.91 (d, J=2.2 Hz, 1H), 4.85 (s, 1H), 4.79 (d, J=2.1 Hz, 1H), 4.69 (d, J=1.91 Hz, 1H), 4.52 (td, J=10.2, 4.51 Hz, 1H), 4.45-4.36 (m, 2H), 4.18 (t, J=4.6 Hz, 1H), 4.16-4.13 (m, 1H), 4.13-4.10 (m, 2H), 4.06 (ddd, J=7.2, 5.9, 3.3 Hz, 1H), 3.99 (dt, J=9.4, 4.7 Hz, 1H), 3.97-3.93 (m, 1H), 3.91 (dd, J=6.6, 4.6 Hz, 1H), 3.82-3.74 (m, 3H), 3.72 (dd, J=10.3, 4.81 Hz, 1H), 3.68 (dd, J=6.6, 3.9 Hz, 1H), 3.53 (s, 3H), 3.46-3.40 (m, 1H), 3.33 (s, 3H), 3.19 (dd, J=13.6, 1.8 Hz, 1H), 3.06-2.95 (m, 2H), 2.62-2.54 (m, 2H), 2.52 (ddd, J=13.9, 9.8, 4.4 Hz, 1H), 2.48-2.43 (m, 1H), 2.34-2.27 (m, 2H). 2.23-2.16 (m, 3H), 2.15-2.09 (m, 2H), 2.08-2.03 (m, 2H), 1.98 (s, 3H), 1.95-1.90 (m, 1H), 1.83-1.69 (m, 5H), 1.59-1.41 (m, 5H), 1.34-1.31 (m, 1H), 1.28-1.24 (m, 1H), 1.05 (s, 9H), 1.00 (s, 9H), 0.91 (d, J=6.4 Hz, 3H), 0.25 (s, 3H), 0.23 (s, 3H), 0.11 (s, 3H), 0.11 (s, 3H)ppm; ¹³C NMR (151 MHz, C₆D₆) δ 171.1, 152,8, 151.2, 144.4, 138.4, 130.2, 128.5, 110.5, 104.8, 104.7, 86.9, 82.4, 81.6, 80.9, 79.4,78.8, 78.7, 77.32, 77.27, 77.0, 75.9, 75.0,74.8, 74.3, 72.2, 68.5, 68.4, 58.8, 57.6, 51.1, 47.3, 44.0, 43.0, 40.7, 39.2, 38.4, 35.9, 35.8, 34.2, 32.4, 32.0, 30.9, 30.7, 30.5, 26.31, 26.28, 21.3, 18.7, 18.5, 18.1, −3.8, −4.4, −5.07, −5.08 ppm; 1RMS (ESI-TOF) m/z: [M+Na]⁺ Calcd. for C₆₀H₉₆O₁₅SSi₂Na⁺ 1167.5901; Found 1167.5897.
(1R)-1,4-Anhydro-6-O-[tert-butyl(diphenyl)silyl]-3,5-dideoxy-3-{[(4-methoxybenzyl)oxy]methyl]-1-prop-2-en-1-yl-L-arabino-hexitol (S15): To a stirred solution of diol 28 (800 mg, 1.82 mmol, 1.0 equiv) in CH₂Cl₂/hexane (50 mL, 4:1, v/v) at 0° C. were added 4-

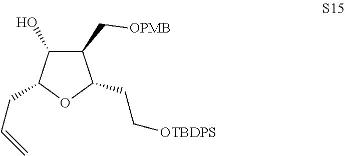

methoxybenzyl-2,2,2-trichloroacetimidate (PMB-TCAI, 375 μL, 1.82 mmol, 1.0 equiv) and (7,7-dimethyl-2-oxobicyclo[22.1]heptan-1-yl)methanesulfonic acid [(±)-CSA; 42.1 mg, 0.182 mmol, 0.1 equiv]. The reaction mixture was allowed to warm to 23° C. and stirred for 4 h before it was quenched by addition of NaHCO₃ solution (10 mL, sat. aq.). The layers were separated, and the aqueous layer was extracted with CH₂Cl₂(3×30 mL). The combined organic layers were washed with brine (30 mL), dried over Na₂SO₄, and concentrated under reduced pressure. Flash column chromatography (SiO₂, CH₂Cl₂/Et₂O 40:1, v/v→10:1, v/v) of the residue afforded alcohol S15 (662 mg, 1.18 mmol. 65% yield) as a colorless oil and recovered diol 28 (100 mg, 0.228 mmol, 13%) as a white foam. 515: R_{f}=0.60 (SiO₂, CH₂Cl₂/Et₂O 9:1, v/v);

$${[ α ]}_{D}^{23} = - 13.3 ( c = 1.5 , {CH}_{2} {Cl}_{2} ) ;$$

FT-IR (film): vₘₐₓ 3435, 2954, 2930, 2856, 1612, 1513, 1427, 1361, 1247, 1110, 1086, 1035, 822, 702 cm⁻¹; ¹H NMR (600 MHz, CDCl₃) δ 7.69-7.65 (m, 4H), 7.44-7.40 (m, 2H), 7.39-7.35 (m, 41H), 7.23 (d, J=8.2 Hz, 2H), 6.86 (d, J=8.2 Hz, 2H), 5.86 (dddd, J=16.3, 10.2, 7.4, 6.5 Hz, 1H), 5.14 (dd, J=17.2, 1,71-Iz, 1H), 5.07 (dd, J=10.2, 1.7 Hz, 1H), 443 (AB quart, J=12.0 Hz, 2H), 4.07 (dd, J=4.1, 2.6 Hz, 1H), 3.86-3.78 (m, 5H), 3.74-3.66 (m, 2H), 3.48 (dd, J=9.3, 5.8 Hz, 1H), 3.42 (dd, J=9.3, 7.5 Hz, 1H), 2.41 (td, J=7.0, 1.3 Hz, 2H), 2.22-2.12 (m, 1H), 1.89 (q, J=62 Hz, 2H), 1.04 (d, J=1.2 Hz, 9H)ppm; ¹³C NMR (151 MHz, CDCl₃) δ 159.3, 135.7, 135.6, 135.2, 133.9, 133.8, 130.3, 129.8, 129.7, 129.3, 127.8, 127.7, 117.0, 113.9, 81.0, 77.3, 76.6, 73.0, 70.1, 61.2, 55.4, 54.3, 38.4, 33.8, 27.0, 19.3 ppm; HRMS (ESI-TOF) mv z: [M+Na]⁺ Calcd. for C₃₄H₄₄O₅SiNa⁺ 583.2850: Found 583.2845.
(1R)-1,4-Anhydro-6-O-[tert-butyl(diphenyl)silyl]-3,5-dideoxy-3-{[(4-methoxybenzyl)oxy]methyl}-2-O-methyl-1-prop-2-en-1-yl-L-arabino-hexitol (S16): To a stirred

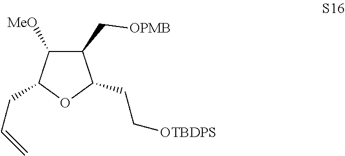

solution of alcohol S15 (550 mg, 0.981 mmol, 1.0 equiv) in THF/DMF (10 mL, 4:1, at 0° C. were added sodium hydride (60% dispersion in mineral oil, 235 mg, 5.88 mmol, 6.0 equiv) and methyl iodide (305 mL, 4.90 mmol, 5.0 equiv). The reaction mixture was allowed to warm to 23° C. and stirred for 1 h before it was carefully quenched by addition of NH₄Cl solution (10 mL, sat. aq.). The layers were separated, and the aqueous layer was extracted with EtOAc (3×20 mL). The combined organic layers were dried over Na₂SO₄ and concentrated under reduced pressure. Flash column chromatography (SiO₂, hexanes/EtOAc 20:1, v/v→2:1, v/v) of the residue afforded olefin S16 (519 mg, 0.902 mmol, 92% yield) as a colorless oil. S16: R_{f}=0.60 (SiO₂, hexanes/EtOAc 2:1, v/v);

$${[ α ]}_{D}^{23} = - 25.3 ( c = 0.6 , {CH}_{2} {Cl}_{2} ) ;$$

FT-IR (film): vₘₐₓ 3049, 2998, 2856, 1613, 1513, 1428, 1248, 1110, 1036, 915, 822, 738, 702 cm¹; ¹H NMR (600 MHz, CDCl₃) δ 7.67-7.64 (m, 4H), 7.43-7.38 (m, 2H), 7.38-7.34 (m, 4H), 7.22 (d, J=8.2 Hz, 2H), 6.86 (d, J=8.2 Hz, 2H), 5.83 (ddt, J=17.2, 10.2, 7.0 Hz, 1H), 5.11 (dd, J=17.2, 1.6 Hz, 1H), 5.03 (dd, J=10.2, 1.6 Hz, 1H), 4.44 (AB quart, J=12.0 Hz, 2H), 3.85-3.75 (m, 5H), 3.70 (td, J=7.3, 5.2 Hz, 1H), 3.64 (td, J=−7.0, 4.1 Hz, 1H), 3.55 (dd, J=4.2, 1.8 Hz, 1H), 3.43 (dd, J=9.4, 5.7 Hz, 1H), 3.36 (dd, J=9.4, 7.7 Hz, 1H), 3.28 (s, 3H), 2.48-2.34 (m, 2H), 2.20-2.13 (m, 1H), 1.97-1.86 (m, 2H), 1.03 (s, 9H) ppm; ¹³C NMR (151 MHz, CDCl₃) δ 159.2, 135.7, 135.6, 135.5, 134.1, 134.0, 130.5, 129.7, 129.6, 129.1, 127.7, 127,6, 116.7, 113.9, 85.1, 81,1, 77.8, 73,0, 70.2, 61.3, 56.8, 55.4, 51,1, 38.7, 33.5, 27.0, 19.4 ppm; HRMS (EST-TOF) m/z: [M+Na]⁺ Calcd. for C₃₅H₄₆O₅SiNa⁺ 5970.3007; Found 597.3004.
3,6-Anhydro-1-O-[tert-butyl(diphenyl)silyl]-2,4,7-trideoxy-4-{[(4-nethoxybenzyl)oxy]methyl}-5-O-methyl-8,9-O-(1-methylethylidene)-D-glycero-D-gulo-nonitol (37): To a stirred solution of olefin

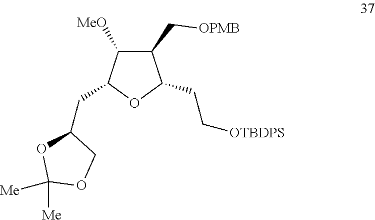

S16 (500 mg, 0.870 mmol, 1.0 equiv) in t-BuOH/H₂O (40 mL, 1:1, v/v) at −5° C. was added AD-mix-α (1.39 g, 1.60 g/mmol of S16) and methane sulfonamide (87.0 mg, 100 mg/mmol of S16). The resulting mixture was stirred at −5° C. for 36 h before it was diluted with water (10 mL). The layers were separated, and the aqueous layer was extracted with EtOAc (3×30 mL). The combined organic layers were dried over Na₂SO₄ and concentrated under reduced pressure to obtain the corresponding diol intermediate.

To the stirred solution of so-obtained crude diol in acetone (10 mL) at 23° C. was added 2,2-di-methoxypropane (533 μL, 4.35 mmol, 5.0 equiv) and p-TsOH·H₂O (33.0 mg, 0.174 mmol, 0.2 equiv). The resulting mixture was stirred for 20 min before it was quenched by addition of NaHCO₃ (10 mL, sat. aq.). The layers were separated, and the aqueous layer was extracted with EtOAc (3×20 mL). The combined organic extracts were dried over Na₂SO₄ and concentrated under reduced pressure. Flash column chromatography (SiO₂, hexanes/EtOAc 10:1, v/v→4:1, v/v) of the residue afforded ketal 37 (423 mg, 0.653 mmol, 75% yield overall) as a colorless oil. 37: R_{f}=0.50 (SiO₂, hexanes/EtOAc 4:1, v/v);

$${[ α ]}_{D}^{23} = - 26. ( c = 1. , {CH}_{2} {Cl}_{2} ) ;$$

FT-IR (film): vₘₐₓ 2931, 2857, 1613, 1587, 1513, 1472, 1428, 1368, 1248, 1110, 1037, 822, 739, 703 cm¹; H NMR (600 MHz, CDCl₃) δ 7.70-7.63 (m, 4H), 7.44-7.39 (m, 2H), 7.40-7.34 (m, 4H), 7.28-7.15 (m, 2H), 6.87 (d, J=8.6 Hz, 2H), 4.54-4.40 (m, 2H), 4.24-4.14 (m, 1H), 4.05 (dd, J=8.0, 5.9 Hz, 1H), 3.85-3.78 (m, 5H), 3.77-3.74 (m, 1H), 3.70 (td, J=7.1, 5.6 Hz, 1H), 3.66-3.55 (m, 2H), 3.49-3.35 (m, 2H), 3.28 (s, 3H), 2.23-2.13 (m, 1H), 2.00 (dt, J=13.5, 6.7 Hz, 1H), 1.97-1.86 (m, 3H), 1.42 (s, 3H), 1.37 (s, 3H), 1.05 (s, 9H)ppm; ¹³C NMR (151 MHz, CDCl₃) δ 159.3, 135.69, 135.67, 134.1, 134.0, 130.4, 129.7, 129.1, 127.73, 127.72, 113.9, 108.7, 85.2, 78.2, 77.81, 73.77, 73.1, 70.2, 69.6, 61.2, 56.6, 55.4, 51.3, 38.6, 32.6, 27.1, 27.0, 26.0, 19.4 ppm; HRMS (ESI-TOF) m/z: [M+Na]⁺ Calcd. for CsH₅₂O₇SiNa⁺ 671.3375; Found 671.3373, 3,6-Anhydro-2,4,7-trideoxy-4-{[(4-methoxybenzyl)oxy]methyl}-5-O-methyl-8,9-O-(1-methyl-ethylidene)-D-glcero-D-gulo-nonitol (S17): To a stirred solution of ketal 37 (300 mg, 0.462 mmol,

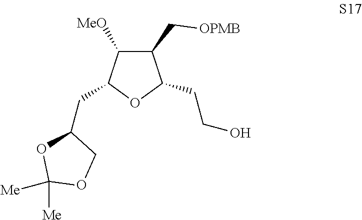

1.0 equiv) in THF (5 mL) at 23° C. was added N,N,Ntributylbutan-1-aminiun fluoride (1.0 M solution in THF 1.02 mL, 1.02 mmol, 2.2 equiv). The resulting mixture was stirred for 2 h before it was quenched by addition of NH₄Cl solution (10 mL, sat. aq.). The layers were separated, and the aqueous layer was extracted with EtOAc (3×20 mL). The combined organic extracts were dried over Na₂SO₄ and concentrated under reduced pressure. Flash column chromatography (SiO₂, hexanes/EtOAc 3:1, v/v→1:4, v/v) of the residue afforded alcohol S17 (236 mg, 0.439 mmol, 95% yield) as a colorless oil. S17: R_{f}=0.60 (SiO₂, 100% EtOAc);

$${[ α ]}_{D}^{23} = - 43.6 ( c = 0.57 , EtOAc ) ;$$

FT-IR (film): vₘₐₓ 3463, 2984, 2993, 2873, 1613, 1586, 1514, 1456, 1369, 1302, 1247, 1173, 1159, 1094, 1063, 1036, 821 cm⁻¹; ¹H NMR (600 MHz, C₆D₆) δ 7.17 (d, J=8.7 Hz, 2H), 6.82 (d, J=8.6 Hz, 2H), 4.26-4.16 (m, 3H), 3.90-3.82 (m, 2H), 3.75 (dq, J=11.1, 6.6 Hz, 2H), 3.66 (ddd, J=7.7, 6.8, 4.3 Hz, 1H), 3.50-3.43 (m, 2H), 3.31 (s, 3H), 3.09 (dd, J=6.9, 3.9 Hz, 2H), 3.06 (s, 3H), 2.20 (qd, J=6.8, 1.8 Hz, 1H), 2.12 (ddd, 1=14.0, 7.6, 6.6 Hz, 1H), 2.03 (ddd, J=13.7, 7.2, 4.9 Hz, 1H), 1.84 (dtd, J=14.5, 7.1, 4.5 Hz, 1H), 1.78 (ddt, J=14.2, 6.4, 4.4 Hz, 1H), 1.43 (s, 3H), 1.35 (s, 3H)ppm; ¹³C NMR (151 MHz, C₆D₆) δ 159.5, 130.3, 129.0, 113.8, 108.6, 84.8, 80.4, 78.9, 73.7, 72.7, 69.9, 69.4, 60.5, 55.8, 54.5, 50.9, 37.6, 32.8, 26.9, 25.8 ppm; HRMS (ESI-TOF) m/z: [M+Na]⁺ Calcd. for C₂₂H₃₄O₇Na⁺ 433.2197; Found 433.2202.
3,6-Anhydro-2,4,7-trideoxy-4-{[(4-methoxybenzyl)oxy]methyl}-5-O-methyl-8,9-O-(1-methylethylidene)-D-glycero-D-gulo-nonose (38): To a stirred solution of alcohol S17 (170 mg,

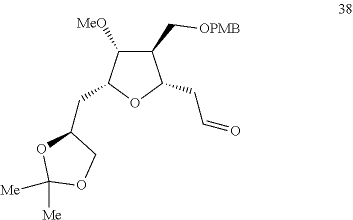

0.316 mmol, 1.0 equiv) in CH₂Cl₂ (10 mL) at 0° C. was added Dess-Martin periodinane (335 mg, 0.790 mmol, 2.5 equiv). The resulting mixture was allowed to warm to 23° C. and stirred for 1 h before it was diluted with NaHCO₃ solution (10 mL, sat. aq.) and Na₂S₂O₃ solution (15 mL, sat. aq.). The resulting mixture was vigorously stirred for 2h. The layers were separated, and the aqueous layer was extracted with CH₂Cl₂ (3×30 mL). The combined organic extracts were dried over Na₂SO₄ and concentrated under reduced pressure. Flash column chromatography (SiO₂, hexanes/EtOAc 3:1, v/v→1:1, v/v) of the residue afforded aldehyde 38 (120 mg, 0.294 mmol, 93% yield) as a white foam. 38: R_{f}=0.60 (SiO₂, hexanes/EtOAc 1:1, v/v);

$${[ α ]}_{D}^{23} = - 36.5 ( c = 0.65 , EtOAc ) ;$$

FT-IR (film): vₘₐₓ 2984, 2933, 2868, 1724, 1613, 1514, 1457, 1369, 1302, 1248, 1160, 1092, 1064, 1034, 822 cm⁻¹; ¹H NMR (600 MHz, C₆D₆): δ 9.52 (t, J=2.1 Hz, 1H), 7.17 (d, J=8.5 Hz, 2H), 6.83 (d, J=8.6 Hz, 2H), 4.28-4.18 (m, 3H), 3.89 (ddd, J=7.3, 6.4, 4.2 Hz, 1H), 3.87-3.84 (m, 2H), 3.50 (dd, J=8.0, 7.3 Hz, 1H), 3.44 (dd, J=4.3, 1.7 Hz, 1H), 3.31 (s, 3H), 3.11 (dd, J=6.9, 2.2 Hz, 2H), 3.00 (s, 3H), 2.47 (ddd, J=16.3, 7.2, 2.4 Hz, 1H), 2.34 (ddd, J=16.3, 5.0, 1.71 Hz, 1H), 2.17 (dt, J=13.6, 6.8 Hz, 1H), 2.12-2.02 (m, 2H), 1.43 (s, 3H), 1.35 (s, 3H)ppm; ¹³CNMR (151 MHz, C₆D₆): δ 199.9, 159.9, 130.6, 129.4, 114.2, 108.8, 85.1, 79.1, 76.3, 73.9, 73.1, 70.0, 69.7, 56.2, 54.8, 51.3, 49.5, 33.1, 27.3, 26.1 ppm; HRMS (ESI-TOF) m/z: [M+Na]⁺ Calcd. for C₂₂H₃₂O₇Na⁺ 431.2040; Found 431.2032.
Methyl [(1S,2S,4R,7S,9R,10S,12S,14R,16R)-12-{2-[(2S,5S)-5-{2-[(2S,4R,6R)-6-{[(2S,3S,4R,5R)-5-{[(4S)-2,2-dimethyl-1,3-dioxolan-4-yl]methyl}-4-methoxy-3-{[(4-methoxybenzyl)oxy]methyl]-tetrahydrofuran-2-yl]methyl}-4-methyl-5-methylidenetetrahydro-2H-pyran-2-y]ethyl}-4-methylidenetetrahydrofuran-2-yl]ethyl}-3,8,11,15,17-pentaoxapentacyclo[10.4.1.0^{2,7}.0^{9,16}.0^{10,14}]heptadec-4-yl]acetate (40): In a glove box, to a stirred

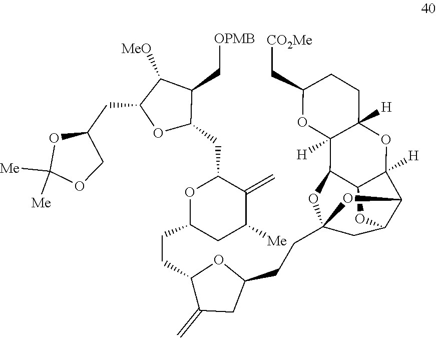

solution of aldehyde 38 (94 mg, 0.23 mmol, 1.5 equiv), fragment IJKLMN¹ (8; 120 mg, 0.15 mmol, 1.0 equiv), Et₃N (110 μL, 0.77 mmol, 5.0 equiv), and CrCl₂ (56 mg, 0.46 mmol, 3.0 equiv) in THF (2 mL) at 23° C. was added a premixed solution of ligand (−)-39 (110 mg, 0.38 mmol, 2.5 equiv) and NiCl₂ (4.0 mg, 0.031 mmol, 0.2 equiv) in THF (1 mL). The resulting mixture was stirred for 10 h before it was quenched by addition of H₂O (10 mL). The layers were separated, and the aqueous layer was extracted with EtOAc (4×30 mL). The combined organic extracts were washed with brine (10 mL), dried over Na₂SO₄, and concentrated under reduced pressure. Flash column chromatography (SiO₂, hexanes/EtOAc 3:1, v/v→1:5, v/v) of the residue afforded the corresponding allyl alcohol (110 mg, 0.110 mmol, 69% yield) as a light-yellow oil.

To a stirred solution of above allyl alcohol (110 mg, 0.110 mmol, 1.0 equiv) in toluene (10 mL) at 23° C. were added DBU (1.0 mL, 6.7 mmol, 63 equiv, Note: toluene:DBU 10:1, v/v). The resulting mixture was heated to 110° C. and stirred for 1 h before it was allowed to cool to 23° C. and concentrated under reduced pressure. Flash column chromatography (SiO₂, hexanes/EtOAc 3:1, v/v→1:5, v/V) of the residue afforded ester 40 (81 mg, 0.086 mmol, 81% yield) as a colorless oil. 40: R_{f}=0.80 (SiO₂, 100% EtOAc);

$${[ α ]}_{D}^{23} = - 47. ( c = 0.6 , EtOAc ) ;$$

FT-IR (film): vₘₐₓ 2932, 2867, 1739, 1612, 1513, 1439, 1369, 1301, 1246, 1210, 1189, 1155, 1133, 1079, 1013, 904, 831 cm⁻¹; ¹H NMR (600 MHz, C₆D₆): δ 7.27 (d, J=8.6 Hz, 2H), 6.86 (d, J=8.6 Hz, 2H), 4.95-4.90 (m, 2H), 4.87 (d, J=2.2 Hz, 1H), 4.75 (d, J=2.01 Hz, 1H), 4.56-4.51 (m, 1H), 4.51 (brs, 1H), 4.45-4.31 (m, 4H), 4.17 (t, J=4.6 Hz, 1H), 4.11 (t, J=4.8 Hz, 1H), 4.05-3.96 (m, 2H), 3.95 (dt, J=6.8, 3.4 Hz, 1H), 3.91 (dt, 1=7.9, 5.1 Hz, 2H), 3.87 (dd, J=8.5, 4.5 Hz, 1H), 3.80-3.72 (m, 1H). 3.68 (dd, J=6.6, 4.0 Hz, 1H), 3.65 (dd, J=4.2, 1.4 Hz, 1H), 3.55 (t, J=7.7 Hz, 1H), 339 (dd, J=9.4, 5.4 Hz, 1H), 3.36 (s, 3H), 3.32 (s, 3H), 3.29-3.22 (m, 2H), 3.20 (s, 3H), 2.62-2.55 (m, 2H), 2.51-2.43 (m, 2¹H), 2.41-2.37 (m, 1H), 2.34 (dt, J=13.6, 6.8 Hz, 1H), 2.27 (ddd, J=13.7, 7.7, 4.6 Hz, 1H), 2.24-2.10 (m, 4H), 2.08-1.98 (m, 3H), 1.96 (d, J=13.1 Hz, 1H), 1.86-1.71 (m, 4H), 1.69-1.62 (m, 1H), 1.58-1.51 (m, 1H), 1.48-1.40 (m, 6H), 1.36 (s, 3H), 1.34-1.22 (m, 2H), 1.10-1.00 (m, 1H), 0.95 (d, J=6.5 Hz, 3H)ppm; ¹³C NMR (151 MHz, CD): δ 171.1, 159.9, 152.9, 151.8, 131.0, 129.4, 114.23, 110.5, 108.7, 104.6, 104.4, 86.2, 82.4, 81.0, 79.5, 78.7, 78.4, 77.3, 77.2, 77.0, 76.0, 75.0, 74.7, 74.4, 74.2, 73.0, 70.5, 69.9, 68.5, 56.3, 54.9, 51.8, 51.1, 47.3, 43.4, 40.7, 39.3, 38.4, 36.1, 35.9, 33.3, 32.3, 32.0, 30.9, 30.7, 30.5, 27.4, 26.2, 18.6 ppm; HRMS (ESI-TOF) m/z: [M+N]⁺ ]⁺ Calcd. for C₅₂H₇₄O₁₅Na⁺ 961.4920; Found 961.4912.
Methyl [(1S,2S,4R,7S,9R,10S,12S,14R,16R)-12-{2-[(2S,5V)-5-{2-[(2S,4R,6R)-6-{[(2S,3S,4R,5R)-5-{[(4S)-2,2-dimnethyl-1,3-dioxolan-4-yl]methyl}-3-(hydroxymnethyl)-4-methoxytetrahydrofuran-2-yl]methyl}-4-methyl-5-methylidenetetrahydro-2H-pyran-2-yl]ethyl}-4-methylidenetetrahydrofuran-2-yl]ethyl}-3,8,11,15,17-

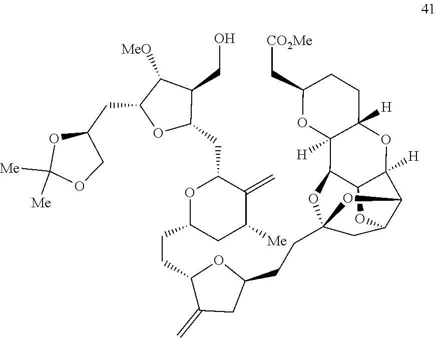 [SMILES: C=C1C[C@H](CC[C@@]23C[C@H]4O[C@H]5[C@@H](O2)[C@H]2O[C@@H](CC(=O)OC)CC[C@@H]2O[C@H]5[C@H]4O3)O[C@H]1CC[C@H]1C[C@@H](C)C(=C)[C@@H](C[C@@H]2O[C@H](C[C@H]3COC(C)(C)O3)[C@H](OC)[C@H]2CO)O1]

pentaoxapentacyclo[10.4.1,0^{2,7}0.^{9,16}.0^{10,14}]heptadec-4-yl]acetate (41): To a stirred solution of ester 40 (70.6 mg, 75.2 mol, 1.0 equiv) in CH₂Cl₂ (10 mL) and aqueous phosphate buffer (1.00 mL, pH 7) at 23° C. was added DDQ (171 mg, 0.752 mmol, 10 equiv) in one portion. After 3 h, the reaction mixture was quenched by addition of NaHCO₃ solution (10 mL, sat. aq.). The layers were separated, and the aqueous layer was extracted with CH₂Cl₂ (3×15 mL). The combined organic extracts were dried over Na₂SO₄ and concentrated under reduced pressure. Flash column chromatography (SiO₂, hexanes/EtOAc 3:1, v/v→1:10, v/v) of the residue afforded alcohol 41 (43.7 mg, 53.4 μmol, 71% yield) and recovered 40 (12.7 mg, 13.5 gmol, 18%) as colorless oils, respectively. 41: R_{f}=0.60 (SiO₂, 100% EtOAc);

$${[ α ]}_{D}^{23} = - 43.3 ( c = 0.3 , EtOAc ) ;$$

FT-IR (film): vₘₐₓ 3455, 2927, 2865, 1736, 1652, 1438, 1369, 1260, 1190, 1133, 1078, 1016, 903, 844 cm⁻¹; ¹H NMR (600 MHz, C₆D₆) δ 4.96 (s, 1H), 4.94 (d, J=2.2 Hz, 1H), 4.91 (d, J=2.2 Hz, 1H), 4.76 (d, J=1.9 Hz, 1H), 4.56-4.48 (m, 2H), 4.42 (dd, J=4.1, 2.0 Hz, 1H) 4.40-4.32 (m, 1H), 4.17 (t, J=4.5 Hz, 1H), 4.11 (t, J=4.7 Hz, 1H), 4.06-3.98 (i, 2H), 3.95-3.88 (m, 41H), 3.76 (dddd, J=9.6, 7.5, 5.4, 2.2 Hz, 1H), 3.68 (dd, J=6.6, 4.0 Hz, 1H), 3.61-3.50 (m, 3H), 3.48-3.42 (m, 2H), 3.34 (s, 3H). 3.15 (s, 3H), 2.64-2.56 (m, 2H), 2.48 (ddd, J=18.4, 7.6, 3.71 Hz, 2H), 2.37-2.29 (m, 2H), 2.28-2.18 (m, 4H), 2.18-2.09 (m, 2H), 2.05 (ddt, J=15.6, 9.2, 2.7 Hz, 2H), 2.01-1.94 (m, 2H), 1.81-1.69 (m, 5H), 1.56-1.61 (m, 1H), 1.48 (dd, J=13.1, 5.1 Hz, 1H), 1.45 (s, 3H), 1.45-1.40 (m, 1H), 1.36 (s, 3H), 1.34-1.25 (m, 2H), 1.08-0.98 (m, 1H), 0.93 (d, J=6.5 Hz, 3H)ppm; ¹³C NMR (151 MHz, C₆D₆) δ 170.8, 152.4, 151.0, 110.1, 108.4, 104.44, 104.39, 85.4, 82.0, 80.6, 78.9, 78.3, 78.1, 78.0, 77.0, 76.9, 76.6, 75.7, 74.6, 74.4, 73.9, 73.8, 69.5, 68.1, 63.0, 55.9, 53.2, 50.8, 47.0, 42.8, 40.4, 38.8, 37.8, 35.6, 35.5, 33.1, 31.8, 31.5, 30.5, 30.3, 30.1, 27.0, 25.8, 17.8 ppm; HRMS (ESI-TOF) m/z: M+Na]⁺ Calcd. for C₄₄H₆₆O₁₄Na⁺ 841.4345; Found 841.4326.

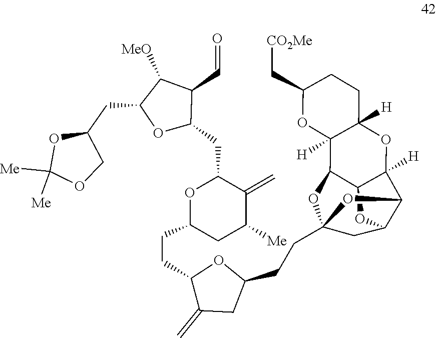 [SMILES: C=C1C[C@H](CC[C@@]23C[C@H]4O[C@H]5[C@@H](O2)[C@H]2O[C@@H](CC(=O)OC)CC[C@@H]2O[C@H]5[C@H]4O3)O[C@H]1CC[C@H]1C[C@@H](C)C(=C)[C@@H](C[C@@H]2O[C@H](C[C@H]3COC(C)(C)O3)[C@H](OC)[C@H]2C=O)O1]

Methyl [(1S,2S,4R,7S,9R,10S,12S,14R,16R)-12-{2-[(2S,5S)-5-{2-[(2S,4R,6R)-6-{[(2S,3S,4R,5R)-5-{[(4S)-2,2-dimethyl-1,3-dioxolan-4-yl]methyl}-3-formyl-4-methoxytetrahydlrofuran-2-yl]methyl}-4-methyl-5-methylidenetetrahydro-2H-pyran-2-yl]ethyl}-4-methylidenetetrahydrofuran-2-yl]ethyl}(3,8,11,15,17-pentaoxapentacyclo[10.4.1.0^{2,7}0.^{9,16}0.^{10,14}]heptadec-4-yl]acetate (42): To a stirred solution of alcohol 41(7.5 mg, 9.2 gmol, 1.0 equiv) in dry CH₂Cl₂ (2 mL) at 23° C. was added Dess-Martin periodinane (10 mg, 23 μmol, 2.5 equiv). The resulting mixture was stirred for 1 h before it was diluted with NaCO₃ (2 mL, sat. aq.) and Na₂S₂O₃ solution (3 mL, sat. aq.). The resulting mixture was vigorously stirred for 0.5h. The layers were separated, and the aqueous layer was extracted with CH₂Cl₂ (3×10 mL). The combined organic extracts were dried over Na₂SOa and concentrated under reduced pressure. Flash column chromatography (SiO₂, hexanes/EtOAc 3:1, v/v→1:5, v/v) of the residue afforded aldehyde 42 (6.8 mg, 8.3 μmol, 90% yield) as a white foam. 42: R_{f}=0.80 (SiO₂, 100% EtOAc);

$${[ α ]}_{D}^{23} = - 3 4.3 ( c = 0.26 , EtOAc ) ;$$

FT-IR (film): vₘₐₓ 2931, 2853, 1739, 1437, 1370, 1260, 1211, 1191, 1134, 1080, 1016, 904 cm¹; ¹H NMR (600 MHz, C₆D₆) δ 9.52 (d, J=3.3 Hz, 1H), 4.97 (d, J=2.2 Hz, 1H), 4.95 (d, J=2.2 Hz, 1H), 4.83 (s, 1H), 4.72 (d, J=1.9 Hz, 1H), 4.56-4.48 (m, 2H), 4.44-4.37 (m, 2H), 4.32-4.26 (m, 1H), 4.17 (t, J=4.5 Hz, 1H), 4.11 (t, J=4.71 Hz, 1H), 4.03 (dq, J=7.9, 5.81 Hz, 1H), 3.94-3.86 (m, 2H), 3.81-3.72 (m, 4H), 3.68 (dd, J=6.6, 4.0 Hz, 1H), 3.54-3.49 (m, JH), 3.38-3.34 (m, 1H), 3.32 (s, 3H), 2.97 (s, 3H), 2.78 (dt, J=8.2, 3.2 Hz, 1H), 2.62-2.55 (m, 2H), 2.52-2.44 (m, 2H), 2.27-2.11 (m, 4H), 2.08-2.01 (m, 4H), 1.99-1.91 (m, 2H), 1.86-1.74 (m, 4H), 1.74-1.67 (m, 1H), 1.56 (ddt, J=10.4, 8.1, 4.9 Hz, 1H), 1.47 (dd, J=13.1, 5.1 Hz, 1H), 1.44 (s, 3H), 1.43-1.39 (m, 1H), 1.36 (s, 3H), 1.31 (dd, J=10.9, 3.2 Hz, 1H), 1.28-1.24 (m, 1H), 1.05-0.96 (m, 1H), 0.91 (d, J=6.4 Hz, 3H)ppm; ¹³C NMR (151 MHz, C₆D₆) δ 199.5, 171.1, 153.0, 151.3, 110.5, 108.8, 104.7 (2 C), 84.1, 824, 81.0, 79.4, 79.4, 78.7, 77.3, 77.1, 77.0, 76.3, 75.3, 75.0, 74.7, 74.4, 739, 69.7, 68.5, 65.0, 56.7, 51,1, 47.3, 42.8, 40.8, 39.2, 37.3, 35.9, 35.8, 33.4, 32.3, 31.9, 30.9, 30.7, 30.5, 27.3, 26.2, 18.1 ppm; HRMS (ESI-TOF) m/z: [M+Na]⁺ Calcd. for C₄₄H₆₄O₁₄Na⁺ 839.4188; Found 839.4183.
Methyl [(1S,2S,4R,7S,9R,10S,12S,14R,16R)-12-{2-[(2S,5S)-5-{2-[(2S,4R,6R)-6-{[(2S,3S,4R,5R)-5-{[(4S)-2,2-dimethyl-1,3-dioxolan-4-ylimethyl}-3-(iodomethyl)-4-methoxytetrahydrofuran-2-yl]-methyl}-4-methyl-5-methylidenetetrahydro-2H-pyran-2-yl]ethyl}-4-methylidenetetrahydrofuran-2-ylethyl-3,8,11,15,17-pentaoxapentacyclo[10.4.1.0^{2,7}.0^{9,16}0.^{10.14}]heptadec-4-yl]acetate (S18): To a stirred solution of

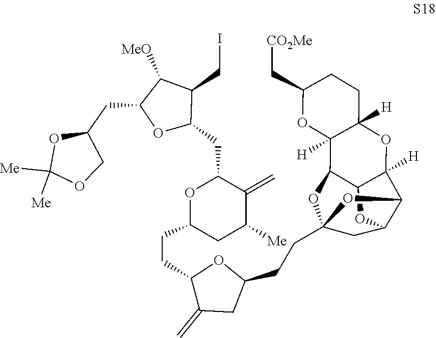 [SMILES: C=C1C[C@H](CC[C@@]23C[C@H]4O[C@H]5[C@@H](O2)[C@H]2O[C@@H](CC(=O)OC)CC[C@@H]2O[C@H]5[C@H]4O3)O[C@H]1CC[C@H]1C[C@@H](C)C(=C)[C@@H](C[C@@H]2O[C@H](C[C@H]3COC(C)(C)O3)[C@H](OC)[C@H]2CI)O1]

aldehyde 41 (32.6 mg, 0.0398 mmol, 1.0 equiv) in THF (5 mL) at 0° C. were added imidazole (13.5 mg, 0.199 mmol, 5.0 equiv), Ph₃P (31.2 g, 0.119 mmol, 3 equiv) and I₂ (25.3 mg, 0.0995 mmol, 2.5 equiv) in one-minute intervals. The resulting mixture was allowed to warm to 23° C. and stirred for 1 h before it was quenched by addition of H₂O (5 mL). The layers were separated, and the aqueous layer was extracted with EtOAc (3×10 mL). The combined organic extracts were washed with NaHCO₃ solution (5 mL, sat. aq.), dried over Na₂SO₄ and concentrated under reduced pressure. Flash column chromatography (SiO₂, hexanes/EtOAc 51, v/v→1:5, v/v) of the residue afforded iodide S18 (34.0 mg, 0.0366 mmol, 92% yield) as a white foam. S18: R_{f}=0.40 (SiO₂, hexanes/EtOAc 1:1, v/v);

$${[ α ]}_{D}^{23} = - 43. ( c = 0.5 , EtOAc ) ;$$

FT-IR (film): vₘₐₓ 2926, 1740, 1437, 1369, 1210, 1192, 1134, 1080, 1018, 902, 751 cm⁻¹; ¹H NMR (600 MHz, C₆D₆): δ 4.96 (d, J=2.2 Hz, 1H), 4.91 (d, J=2.2 Hz, 1H), 4.89 (s, 1H), 4.75 (d, J=1.9 Hz, 1H), 4.56-4.51 (m, 2H), 4.42 (dd, 1=4.0, 1.9 Hz, 1H), 4.28 (p, J=6.4 Hz, 1H), 4.17 (t, J=4.5 Hz, 1H), 4.11 (t, J=4.7 Hz, 1H), 4.02 (dq, J=8.0, 6.0 Hz, 1H), 3.94-3.88 (m, 2H), 3.87-3.79 (m, 3H), 3.77 (dddd, J=9.7, 7.3, 5.3, 2.1 Hz, 1H), 3.68 (dd, J=6.6, 3.9 Hz, 1H), 3.55 (t, J=7.6 Hz, 1H), 3.41 (dd, J=4.0, 1.5 Hz, 1H), 3.36 (ddq, J=9.7, 5.9, 2.21 Hz, 1H), 3.32 (s, 3H), 3.15 (s, 3H), 3.11-3.03 (m, 1H), 2.69 (dd, J=10.2, 9.1 Hz, 1H), 2.63-2.54 (m, 2H), 2.52-2.43 (m, 1H), 2.38-2.31 (m, 2H), 2.26 (dt, J=13.5, 6.7 Hz, 1H), 2.22-2.12 (m, 4H), 2.12-2.02 (m, 3H), 2.00-1.91 (m, 2H), 1.87-1.72 (in. 4H), 1.68 (tdd, J=12.5, 6.1, 2.2 Hz, 1H), 1.60-1.53 (m, 1H), 1.48 (dd, 1=13.1, 5.1 Hz, 1H), 1.45 (s, 3H), 1.44-1.40 (m, 2H), 1.36 (s, 3H), 1.31 (dd, J=11.1, 3.1 Hz, 1H), 1.28-1.24 (m, 1H), 1.07-0.98 (m, 1H), 0.92 (d, J=6.5 Hz, 3H)ppm; ¹³C NMR (151 MHz, C₆D₆): δ 170.8, 152.5, 151.1, 110.1, 108.5, 104.31, 104.28, 87.7, 82.0, 81.1, 80.6, 79.1, 78.3, 77.6, 77.2, 77.0, 76.7, 75.6, 74.6, 74.4, 74.0, 73.6, 69.3, 68.2, 56.3, 52.6, 50.8, 47.0, 42.8, 40.4, 38.9, 37.3, 35,7, 35.6, 32.8, 32.0, 31.7, 30.5, 30.4, 30.2, 27.0, 25.8, 17.8, 7.2 ppm; HRMS (ESI-TOF) m/z: [M+Na]⁺ Calcd. for C₄₄H₆₅IO₁₃Na⁺ 951.3362; Found 951.3341.
[(1S,2S,4R,7S,9R,10S,12S,14R,16R)-12-{2-[(2S,5S)-5-{2-[(2S,4R,6R)-6-{[(22S,3S,4R,5R)-5-{[(4S)-2,2-Dimethyl-1,3-dioxolan-4-y]methyl}-3-(iodomethyl)-4-methoxytetrahydrofuran-2-yl]methyl}-4-methyl-5-methylidenetetrahydro-21-pyran-2-yl]ethyl}-4-methylidenetetrahydrofuran-2-ylethyl-3,8,11,15,17-pentaoxapentacyclo[10.4.1.0^{2,7}0.^{9,16},0^{10,14}]heptadec-4-yl]acetaldehyde (44): To a stirred solution of

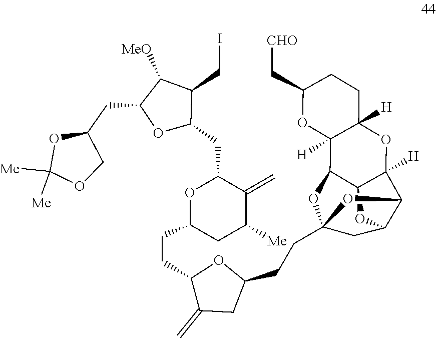 [SMILES: C=C1C[C@H](CC[C@@]23C[C@H]4O[C@H]5[C@@H](O2)[C@H]2O[C@@H](CC=O)CC[C@@H]2O[C@H]5[C@H]4O3)O[C@H]1CC[C@H]1C[C@@H](C)C(=C)[C@@H](C[C@@H]2O[C@H](C[C@H]3COC(C)(C)O3)[C@H](OC)[C@H]2CI)O1]

iodide S18 (29.6 mg, 0.0319 mmol, 1.0 equiv) in CH₂Cl₂ (10 mL) at −78° C. was added DIBAL-H (1.0 M in toluene, 63.8 μL, 0.0638 mmol, 2.0 equiv). The resulting mixture was further stirred at −78° C. for 1 h before it was diluted with EtOAc (0.5 mL) and Rochelle salt solution (10 mL, sat. aq.). The mixture was allowed to warm to 23° C., and vigorously stirred for 1 h. The layers were separated, the aqueous layer was extracted with CH₂Cl₂ (3×10 mL). The combined organic extracts were dried over Na₂SO₄ and concentrated under reduced pressure. Flash column chromatography (SiO₂, hexanes/EtOAc 5:1, v/v→1:5, v/v) of the residue afforded iodoaldehyde 44 (20.3 mg, 0.0226 mmol, 71% yield) as a colorless oil, and recovered iodide S18 (4.50 mg, 0.00479 mmol, 15%) as a white foam. 44: R_{f}:=0.30 (SiO₂, hexanes/EtOAc 1:1, v/v);

$${[ α ]}_{D}^{23} = - 46. ( c = 0.6 , EtOAc ) ;$$

FT-IR (film): vₘₐₓ 2928, 2854, 1726, 1455, 1369, 1190, 1134, 1085, 899, 831 cm⁻¹; ¹H NMR (600 MHz, C₆D₆) δ 9.48 (t, J=2.0 Hz, 1H), 4.96 (d, J=2.2 Hz, 1H), 4.93-4.87 (m, 2H), 4.75 (d, J=1.9 Hz, 1H), 4.51 (dd, J=10.0, 4.2 Hz, 2H), 4.38 (dd, J=4.0, 1.9 Hz, 1H), 4.29 (p, J=6.4 Hz, 1H), 4.18 (t, J=4.5 Hz, 1H), 4.13 (t, J=4.7 Hz, 1H), 4.02 (dq, J=7.9, 6.2 Hz, 1H), 3.96-3.89 (m, 2H), 3.88-3.78 (m, 3H), 3.72 (dd, J=6.5, 3.9 Hz, 1H), 3.55 (L, J=7.7 Hz, 1H), 3.46-3.39 (m, 214H), 3.39-3.34 (m, 1H), 3.15 (s, 3H), 3.10-3.04 (m, 1H), 2.70 (dd, J=10.3, 9.1 Hz, 1H), 2.53-2.45 (m, 2H), 2.37-2.28 (m, 3H), 2.28-2.23 (m, 1H), 2.18-2.12 (m, 3H), 2.11-2.01 (m, 4H), 1.99 (d, J=13.2 Hz, 1H), 1.93 (ddd, J=16.5, 4.6, 1.7 Hz, 2H), 1.88-1.81 (m, 2H), 1.80-1.74 (m, 2H), 1.73-1.64 (m, 1H), 1.57 (ddt, J=9.7, 7.1, 4.5 Hz, 1H), 1.49 (dd, J=13.2, 5.0 Hz, 1H), 1.45 (s, 3H), 1.42 (dd, J=4.5, 2.1 Hz, 1H), 1.36 (s, 3H), 1.28-1.24 (m, 1H), 1.18-1.13 (m, 1H), 1.07-0.98 (m, 1H), 0.93 (d, J=6.5 Hz, 3H)ppm; ¹³C NMR (151 MHz, C₆D₆) δ 199.2, 152.4, 151.1, 110.2, 108.5, 104.4, 104.3, 87.7, 82.0, 81.1, 80.6, 79.2, 78.4, 77.6, 77.1, 76.9, 76.7, 75.6, 74.5, 74.0, 73.7, 73.1, 69.3, 68.1, 56.3, 52.6, 48.8, 47.0, 42.8, 38.9, 37.3, 35.7, 35.6, 32.8, 32.0, 31.7, 30.6, 30.3, 30.2, 27.0, 25.8, 17.8, 7.2ppn; HRMS (ESI-TOF) m/z: [M+Na]⁺ Calcd. for C₄₃H₆₃IO₁₂Na⁺ 921.3256: Found 921.3251.
2-{[tert-Butyl(dimethyl)silyl]oxy}-3-[(1S,2S,4R,7S,9R,10S,12S,14R,16R)-12-{2-[(2S,5V)-5-{2-[(2S,4R,6R)-6-{I(2S,3S,4R,5R)-5-{[(4S)-2,2-dimnethyl-1,3-dioxolan-4-yl]methyl}-3-(iodonethyl)-4-methoxytetrahvdrofuran-2-yl]methyl}-4-methyl-5-methylidenetetrahydro-2H-pyran-2-yl]ethyl}-

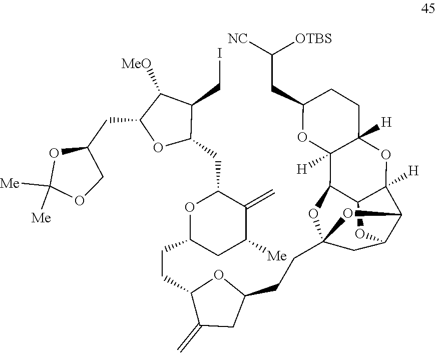 [SMILES: C=C1C[C@H](CC[C@@]23C[C@H]4O[C@H]5[C@@H](O2)[C@H]2O[C@@H](CC(C#N)O[Si](C)(C)C(C)(C)C)CC[C@@H]2O[C@H]5[C@H]4O3)O[C@H]1CC[C@H]1C[C@@H](C)C(=C)[C@@H](C[C@@H]2O[C@H](C[C@H]3COC(C)(C)O3)[C@H](OC)[C@H]2CI)O1]

4-methylidenetetrahydrofuran-2-yl]ethyl}-3,8,11,15,17-pentaoxapentacyclo[10.4.1.0^{2,7}.0^{9,16}.0^{10,14}]heptadec-4-yl]propanenitrile (45): To a stirred solution of iodoaldehyde 44 (3.5 mg, 4.0 gmol, 1.0 equiv) in CH₂Cl₂ (2 mL) at 23° C. were added KCN (0.26 mg, 4.0 μmol, 1.0 equiv), TBSCN (2.8 mg, 20 μmol, 5.0 equiv) and 18-crown-6 (1.1 mg, 0.80 μmol, 1.0 equiv). The resulting mixture was stirred for 2 h before it was quenched by addition of NaHCO₃ solution (3 mL, sat. aq.). The layers were separated, and the aqueous layer was extracted with CH₂Cl₂ (3×5 mL). The combined organic extracts were dried over Na₂SO₄ and concentrated under reduced pressure. Flash column chromatography (SiO₂, hexanes/EtOAc 3:1, v/v→1:5, v/v) of the residue afforded TBS-protected cyanohydrin 45 (mixture of isomers, ~4:1 dr, 3.6 mg, 3.5 μmol, 87% yield) as a colorless oil. 45 (mixture of isomers): R_{f}=0.40 (SiO₂, hexanes/EtOAc 2:1, v/v);

$${[ α ]}_{D}^{23} = - 48.9 ( mixture of isomers , c = 0.19 , {CHCl}_{3} ) ;$$

FT-IR (film): vₘₐₓ 2929, 2858, 1458, 368, 1261, 1188, 1134, 1085, 1016, 838, 784 cm¹; ¹H NMR (600 MHz, C₆D₆, mixture of isomers): δ 4.97-4.94 (m, 1.451), 4.91-4.88 (m, 1.26H), 4.79-4.72 (m, 1.39H), 4.63-4.58 (m, 0.5H), 4.58-4.53 (m, 0.97H), 4.53-4.49 (m, 1.17H), 4.47 (dd, J=10.3, 4.4 Hz, 034H), 4.38 (dd, J=4.0, 1.9 Hz, 0.39H), 4.33-4.24 (m, 0.95H), 4.21 (t, J=4.5 Hz, 0.56H), 4.16 (q, 7=4.2 Hz, 1H), 4.11 (t, J=4.7 Hz, 0.39H), 4.05-3.95 (m, 1H), 3.95 (dd, J=6.6, 4.6 Hz, 0.64H), 3.94-3.86 (m, 1.45H), 3.87-3.77 (m, 3H), 3.66 (dd, J=6.6, 3.9 Hz, 0.40H), 3.59-3.51 (m, 0.95H), 3.51-3.45 (m, 0.54H), 3.44-3.38 (m, 1H), 3.39-3.30 (m, 1.46H), 3.16 (s, 1.56H), 3.14 (s, 1.18H), 3.09 (dd, J=10.2, 4.8 Hz, 0.61H), 3.07-3.03 (m, 0.50H), 2.75-2.70 (m, 0.591H), 2.69-2.65 (m, 0.84H), 2.56 (dd, J=9.6, 2.01 Hz, 0.391), 2.52-2.43 (m, 0.91H), 2.40-2.30 (m, 1.86H), 2.30-2.18 (m. 1.65H), 2.19-2.10 (m, 2H), 2.12-1.98 (m, 4H), 1.98-1.89 (m, 2H), 1.88-1.78 (m, 2H), 1.80-1.65 (m, 4H), 1.61-1.53 (m, 1H), 1.53-1.47 (m, 1H), 1.45 (apparent d, 3H), 1.36 (apparent d, 3H), 1.20-1.10 (m, 2H), 1.09-1.00 (m, 1H), 0.93 (d, J=6.5 Hz, 3H), 0.91 (s, 4H), 0.88 (s, 51H), 0.13-0.02 (m, 6H)ppm; ¹³C NMR (151 MHz, C₃D₆, mixture of isomers): δ 152.8, 152.6, 151.4, 151.3, 120.7, 120.2, 110.7, 110.5, 108.83, 108.80, 104.9, 104.73, 104.66, 88.1, 88.0, 82.43, 82.41, 81.5, 81.1, 80.9, 79.7, 79.6, 78.8, 78.6, 78.99, 77.96, 77.5, 77.33,77.28, 77.09, 76.99, 76.0, 75.9, 75.0, 74.9, 74.5, 74.4, 74.3, 74.04, 73.99, 72.4, 69.72, 69.70, 68.7, 68.5, 60.5, 58.3, 56.7, 53.0, 52.9, 47.4, 47.2, 43.19, 43.15, 42.6, 42.3, 39.4, 37.7, 37.6, 36.04, 36.01, 35.97, 35.91, 33.2, 32.44, 32.37, 32.08, 32.06, 31.2, 31.0, 30.8, 30.7, 30.6, 30.5, 27.38, 27.36, 26.16, 26.15, 25, 8, 25.7, 18.3, 18.2, 18.1, 7.6, 7.5, −5.1, −5.2, −5.3, −5.5 ppm; HRMS (ESI-TOF) m/z: [M+Na]⁺ Calcd. for C₅₀H₇₈INO₁₂SiNa⁺ 1062.4230; Found 1062.4214.
(1S,3S,6S,9S,12S,14R,16R,18S,20R,21R,22S,26R,29S,31R,32S,33R,35R,36S)-20-{[(4S)-2,2-Dimethyl-1,3-dioxolan-4-yl]methyl}-21-methoxy-14-methyl-8,15-dinethylidene-2,19,30,34,37,39,40,41-octa-oxanonacyclo[24.9.2.1^{3,32}.1^{3,33}.1^{6,9}.1^{12,16}0.^{18,22}.0^{29,36}0.^{31,35}] hentetracontan-24-ol (47): In a glove box, to

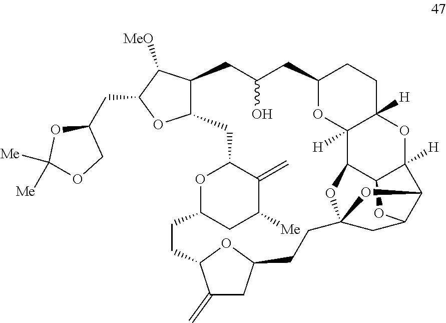 [SMILES: C=C1C[C@@H]2CC[C@@]34CC5O[C@H]6[C@@H](O3)[C@H]3O[C@H](CC[C@@H]3O[C@H]6[C@H]5O4)CC(O)C[C@@H]3[C@@H](OC)[C@@H](C[C@H]4COC(C)(C)O4)O[C@H]3C[C@H]3O[C@@H](CC[C@@H]1O2)C[C@@H](C)C3=C]

a stirred solution of iodoaldehyde 44 (12 mg, 0.013 mmol, 1.0 equiv) in DMF (22 mL) at 23° C. were added cobalt(II) phthalocyanine (7.2 mg, 0.013 mmol, 1.0 equiv), 11(43 ng, 0.26 mmol, 20 equiv) and CrCl₂ (0.16 g, 13 mmol, 100 equiv). The resulting mixture was stirred for 48 h before it was quenched by addition of H₂O (30 mL). The layers were separated, and the aqueous layer was extracted with EtOAc (3×50 mL). The combine organic extracts were dried over Na₂SO₄ and concentrated under reduced pressure. Flash column chromatography (SiO₂, hexanes/EtOAc 10:1, v/v→1:5, v/v) of the residue afforded macrocycle 47 (6.7 mg, 0.0087 mmol, 67% yield) as a white foam. 47: R'₂ 0.50 (SiO₂, 100% EtOAc);

$${[ α ]}_{D}^{23} = - 26.7 ( c = 0.15 , EtOAc ) ;$$

FT-IR (film): vₘₐₓ 3502, 2924, 2851, 1438, 1370, 1134, 1071, 1048, 1021, 991, 974 cm¹; ¹H NMR (600 MHz, CD): 64.96 (s, 1H), 4.93 (d, J=2.2 Hz, 1H), 4.86 (d, J=2.3 Hz, 1H), 4.74 (d, J=1.8 Hz, 1H), 4.66 (d, J=10.3 Hz, 1H), 4.50 (ddd, J=11.1, 9.5, 4.2 Hz, 1H), 4.42 (tt, J=7.2, 5.9 Hz, 1H), 4.33 (dd, J=−4.1, 2.0 Hz, 1H), 4.25 (d, J=6.7 Hz, 1H), 4.19 (dd, J=8.8, 3.2 Hz, 1H), 4.17-4.13 (m, 2H), 4.13-4.08 (m, 2H), 4.00-3.93 (m, 2H), 3.92 (dd, J=6.6, 4.6 Hz, 1H), 3.77 (dd, J=3.7, 1.7 Hz, 2H), 3.70 (dd, J=6.6, 4.1 Hz, 1H), 3.63 (t, J=7.6 Hz, 1H), 3.51-3.45 (m, 1H), 3.33 (s, 3H), 3.05 (d, J=32 Hz, 1H), 2.85-2.79 (m, 1H), 2.66-2.59 (m, 1H), 2.54 (dd, J=9.5, 2.1 Hz, 1H), 2.45-2.34 (m, 4H), 2.27-2.19 (m, 2H), 2.14 (dd, J=15.8, 4.1 Hz, 1H), 2.06-2.01 (m, 1H), 2.00-1.89 (m, 4H), 1.88-1.81 (m, 1H), 1.59 (ddd, J=13.7, 5.8, 3.8 Hz, 3H), 1.55-1.50 (m, 3H), 1.47 (s, 3H), 1.45-1.38 (m, 4H), 1.37 (s, 3H), 1.29 (ddd, J=12.7, 8.3, 3.2 Hz, 1H), 1.19 (dq, J=10.1, 3.1 Hz, 114), 1.08-0.99 (m, 1H), 0.87 (d, J=6.4 Hz, 3+H)ppm; ¹³C NMR (151 MHz, C₆D₆): δ 153.7, 152.7, 110.2, 108.8, 104.1, 104.0, 88.5, 84.1, 82.3, 81.2, 78.1, 77.7, 77.00 (2 C), 76.95, 75.6, 75.4, 75.3, 74.7, 74.4, 74.2, 69.9, 68.8, 68.2, 56.8, 47.6, 44.6, 43.5, 42.9, 42.1, 39.8, 37.4, 36.2, 36.1, 33.9, 329, 31.3, 30.7, 30.6, 30.1, 27.4, 26.2, 18.2 ppm; HRMS (ESI-TOF) m/z: [M+Na]⁺ Calcd. for C₄₃H₆₄O₁₂Na⁺ 795.4290; Found 795.4278.
(1S,3S,6S,9S,12S,14R,16R,18S,20R,21R,22S,26R,29S,31R,32S,33R,35R,36S)-20-{[[(4S)-2,2-Dimethyl-1,3-dioxolan-4-yl]mrethyl}-21-methoxy-14-methyl-8,15-dimethylidene-2,19,30,34,37,39,40,41-octa-oxanonacyclo[24.9.2.1^{3,32}.1^{3,33}.1^{6,9}.1^{12,16}.0^{18,22}.0^{29,36}.0^{31,35}hentetracontan-24-one (S19): To a stirred

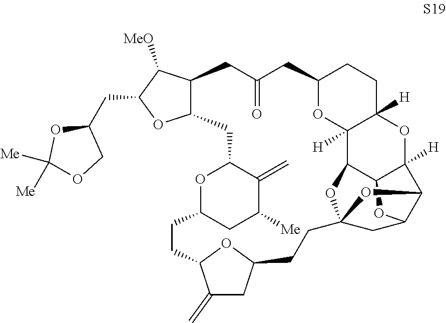 [SMILES: C=C1C[C@@H]2CC[C@@]34CC5O[C@H]6[C@@H](O3)[C@H]3O[C@H](CC[C@@H]3O[C@H]6[C@H]5O4)CC(=O)C[C@@H]3[C@@H](OC)[C@@H](C[C@H]4COC(C)(C)O4)O[C@H]3C[C@H]3O[C@@H](CC[C@@H]1O2)C[C@@H](C)C3=C]

solution of alcohol 47 (5.7 mg, 7.4 mmol, 1.0 equiv) in CH₂Cl₂(3 mL) at 23° C. was added Dess-Martin periodinane (16 mg, 37 mol, 5.0 equiv). The reaction mixture was stirred for 0.5 h before it was quenched by addition of NaHCO₃ (2 mL, sat. aq.) and Na₂S₂O₃ solution (2 mL, sat. aq.). The resulting mixture was vigorously stirred for 1 h. The layers were separated, and the aqueous layer was extracted with CH₂Cl₂ (3×5 mL). The combined organic extracts were dried over Na₂SO₄ and concentrated under reduced pressure. Flash column chromatography (SiO₂, hexanes/EtOAc 5:1, v/v→1:5, v/v) of the residue afforded ketone S19 (5.2 mg, 6.7 mmol, 90% yield) as a white foam. S19: R_{f}=0.80 (SiO₂, 100% EtOAc);

$${[ α ]}_{D}^{23} = - 58. ( c = 0.15 , {CH}_{2} {Cl}_{2} ) ;$$

FT-IR (film): vₘₐₓ 2927, 2854, 1723, 1673, 1438, 1247, 1191, 1134, 1081, 1049, 1021, 991, 903, 831 cm⁻¹; ¹H NMR (600 MHz, C₆D₆) δ 4.96 (d, J=2.2 Hz, 1H), 4.91 (d, J=2.4 Hz, 1H), 4.78 (s, 1H), 4.60 (d, J=1.8 Hz, 1H), 4.58 (d, J=10.9 Hz, 1H), 4.44-4.37 (m, 1H), 4.32 (p, J=6.5 Hz, 1H), 4.22 (qd, J=5.8, 2.9 Hz, 2H), 4.12 (q, J=4.2 Hz, 2H), 4.09-4.03 (m, 2H), 3.97 (t, J=10.7 Hz, 1H), 3.89 (dd, J=8.0, 6.0 Hz, 1H), 3.85 (dd, J=6.6, 4.5 Hz, 1H), 3.71-3.62 (m, 2H), 3.59 (s, 3H), 3.58-3.51 (m, 3H), 2.91-2.86 (m, 1H), 2.76-2.69 (m, 1H), 2.61-2.51 (m, 41H), 2.50-2.45 (m, 1H), 2.41-2.35 (m, 2H), 2.32 (t, J=6.7 Hz, 21), 2,24-2.16 (m, 2H), 2.13 (d, J=16.2 Hz, 1H), 2.07-1.97 (m, 3H), 1.91 (d, J=12.9 Hz, 1H), 1.73-1.60 (m, 4H), 1.55 (dd, J=16.8, 2.0 Hz, 1H), 1.44 (s, 3H), 1.38 (dd, J=13.0, 5.0 Hz, 1H), 1.34 (s, 3H), 1.32-1.28 (m, 1H), 1.25 (t, J=3.0 Hz, 1H), 1.19-1.11 (m, 2H), 1.02-0.93 (m, 1H), 0.81 (d, J=6.5 Hz, 3H)ppm; ¹³C NMR (151 MHz, C₆D₆) δ 205.5, 154.0, 151.8, 109.6, 109.0, 104.5, 104.3, 87.3, 82.6, 81.2, 81.0, 782, 77.8, 76.8 (2 C), 75.3, 74.9, 74.2, 74.0, 73.7, 73.4, 73.1, 70.0, 68.6, 57.0, 48.5 (2 C), 48.2, 44.0, 43.9, 39.5, 39.3, 36.0, 35.4, 33.5, 32.3, 31.3, 31.0, 30.9, 29.1, 27.4, 26.2, 18.0 ppm; HRMS (ESI-TOF) m/z: [M+Na]⁺ Calcd. for C₄₃H₆₂O₁₂Na⁺ 793.4133; Found 793.4116.
(1S,3S,6S,9S,12S,14R,16R,18S,20R,21R,22S,26R,29S,31R,32S,33R,35R,365V)-20-[(2S)-2,3-Dihydroxypropyl]-21-methoxy-14-methyl-8,15-dimethylidene-2,19,30,34,37,39,40,41-octaoxanonacyclo[24.9.2.1^{3,32}.1^{3,33}.1^{6,9}.1^{12,16}.0^{18,22}.0^{29,36}.0^{31,35}]hentetracontan-24-one (48): A solution of ketone S19 (4.7 mg, 6.1 μmol, 1.0 equiv) in AcOH/H₂O (0.3 nL, 2:1, v/v) at 50° C. was stirred 1 h

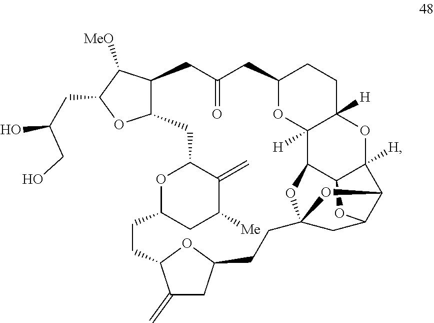

before it was concentrated under reduced pressure. Flash column chromatography (SiO₂, EtOAc:MeOH 100:1, v/v→10:1, v/v) of the residue afforded diol 48 (4.0 mg, 5.5 pnol, 90% yield) as a colorless oil. 48: R_{f}=0.50 (SiO₂, EtOAc/MeOH 10:1, v/v);

$${[ α ]}_{D}^{23} = - 51.8 ( c = 0.11 , EtOAc ) ;$$

FTIR (film): vₘₐₓ 3462, 2925, 2852, 1718, 1652, 1437, 1376, 1263, 1192, 1134, 1081, 1044, 1022, 991, 831 cm⁻¹; ¹H NMR (600 MHz, CDCl₃): δ 5.08 (d, J=2.21 Hz, 1H), 4.94 (d, J=1.91 Hz, 1H), 4.89 (s, 1H), 4.81 (d, J=1.81 Hz, 1H), 4.70 (t, J=4.6 Hz, 1H), 4.61 (t, J=4.5 Hz, 1H), 4.38-4.32 (m, 2H), 4.30 (td, J=10.3, 4.2 Hz, 1H), 4.19 (dd, J=6.6, 4.5 Hz, 1H), 4.15-4.09 (m, 1H), 4.04 (dd, J=6.6, 4.2 Hz, 1H). 4.00-3.90 (m, 3H), 3.85 (dt, J=9.6, 3.2 Hz, 1H), 3.67-3.59 (m, 3H), 3.55 (dd, J=11.3, 5.5 Hz, 1H), 3.44 (s, 3H), 3.28 (d, J=3.2 Hz, 1H), 2.91-2.84 (m, 2H), 2.72 (dd, J=16.1, 10.1 Hz, 1H), 2.55-2.41 (m, 3H), 2.32 (d, 1=16.5 Hz, 1H), 2.30-2.26 (m, 1H), 2.25-2.14 (m, 5H), 2.12-2.05 (m, 1H), 2.03-1.90 (m, 41H), 1.62-1.56 (m, 2H), 1.49-1.43 (m, 2H), 1.40-1.34 (m, 1H), 1.35-1.29 (m, 1H), 1.14-1.05 (in. 4H)ppm; for a comparison with Kishi's data³ see page S108; ¹³C NMR (151 MHz, CDCl₃: δ 207.0, 153,0, 151.1, 109.8, 105.2, 104.2, 87.9, 82.5, 81.29, 81.26, 795, 77.5, 76.6, 76.1, 75.0, 745, 74.0, 73.9, 73.8, 73.7, 70.9, 68, 66.8, 57.4, 48.4, 48.3, 47.7, 43.7, 43.7, 39.1, 38.4, 35.8, 35.0, 32.4, 31.9, 31.1, 30.3, 29.6, 28.4, 18.2 ppm; HRMS (ESI-TO) m/z: [M+Na]⁺ Calcd. for C₄₀H₅₈O₁₂Na⁺ 753.3820; Found 753.3808.
(2R,3R,3aS,7R,8aS,9S,10aR,11S,12R,13aR,13bS,15S,18S,21S,24S,26R,28R,29aS)-2-[(2S)-3-Amino-2-hydroxypropyl]hexacosahydro-3-methoxy-26-methyl-20,27-bis(methylene)-11,15:18,21:24,28-triepoxy-7,9-ethano-12,15-methano-9H,15H-furo[3,2-i]furo[2′,3′:5,6]pyrano[4,3-b][1,4]dioxacyclopentacosin-5(4TH-one (3): Slight modification of Eisai's procedure: To a stirred solution of diol 48 (3.8 mg, 5.2 μmol, 1.0 equiv) in CH₂Cl₂ (300 μL) at

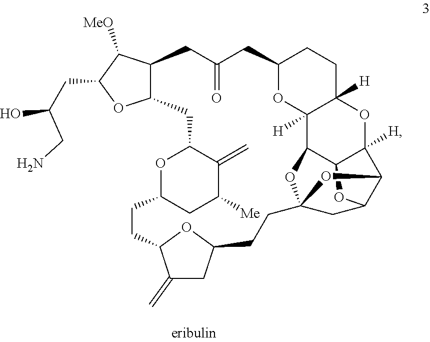

−10° C. were added 2,4,6-collidine (2.8 μL, 21 μmol, 4.0 equiv), pyridine (0.1M in CH₂Cl₂, 5.2 μL, 0.52 μnol, 0.1 equiv), and Ts₂O (1.9 mg, 5.7 μmol, 1.1 equiv) at three-min intervals. The reaction mixture was stirred for 2 h before it was allowed to warm to 0° C. and diluted with a mixture of i-PrOH/NH₄OH (1.5 mL, 1:1, v/v). The mixture was warmed to 30° C. and stirred for 20 h before it was concentrated under reduced pressure. The obtained residue was diluted with NaHCO₃ solution (5 mL, sat. aq.) and extracted with EtOAc (3×10 mL). The combined organic extracts were dried over Na₂SO₄ and concentrated under reduced pressure. Flash column chromatography (C18 RP, H₂O:MeCN 1:1, v/v→1:2, v/v) of the residue afforded eribulin (3; 2.6 mg, 3.5 μmol, 68% yield) as a white amorphous solid. 3: R_{f}=0.50 (SiO₂, MeOH/NH₄OH 20:1, v/v);

$${[ α ]}_{D}^{23} = - 85.3 ( c = 0.15 , MeOH ) ;$$

FTIR (film): vₘₐₓ 3496, 2926, 2847, 1718, 1363, 1192, 1132, 1084, 1045, 1021, 993, 901 cm⁻¹; ¹H NMR (600 MHz, CD₃OD): δ 5.14 (d, J=2.2 Hz, 1H), 5.03 (s, 1H), 4.87 (s, 1H), 4.83 (d, J=1.8 Hz, 1H), 4.71 (t, J=4.5 Hz, 1H), 4.62 (t, J=4.4 Hz, 1H), 4.49 (d, J=11.0 Hz, 1H), 4.33-4.26 (m, 2H), 4.18 (dd, J=6.6, 4.4 Hz, 1H), 4.14-4.07 (m, 2H), 3.99 (t, J=10.81 Hz, 1H), 3.88-3.85 (m, 3H), 3.73 (d, J=11.5 Hz, 1H), 3.72-3.65 (m, 1H), 3.42 (s, 3H), 3.35 (d, J=3.1 Hz, 1H), 2.93 (dd, J=9.7, 2.4 Hz, 1H), 2.91-2.84 (m, 1H), 2.78-2.60 (m, 4H), 2.49-2.41 (m, 2H), 2.40-2.35 (m, 2H), 2.35-2.29 (m, 1H), 2.22-2.15 (m, 1H), 2.13-2.07 (m, 2H), 2.06-1.95 (m, 3H), 1.90-1.81 (m, 3H), 1.80-1.72 (m, 2H), 1.74-1.68 (m, 1H), 1.56 (td, J=12.9, 5.0 Hz, 1H), 1.52-1.35 (m, 4H), 1.34-1.31 (m, 1H), 1.11 (d, J=6.5 Hz, 3H), 1.07-0.98 (m, 1H)ppm; ¹³C NMR (151 MHz, CD₃OD): δ 208.1, 154.4, 153.1, 111.1, 105.1, 105.0, 88.0, 84.0, 82.5, 82.1, 79.2, 78.5, 78.0, 77.8, 75.9, 75.5, 75.3, 74.7, 74.3, 74.2, 70.8, 69.8, 57.3, 49.2, 49.1, 48.5, 47.9, 45.0, 44.8, 40.0, 39.6, 36.8, 36.3, 34.5, 327, 32.1, 31.3, 31.2, 29.8, 18.4 ppm; HRMS (ESI-TOF) m/z: [M+H]⁺ Calcd. for C₄₀H₅₉NO₁₁⁺ 730.4161; Found 730.4148.
Methyl 3,7:6,10-dianhydro-12-O-[tert-butyl(diphenyl)silyl]-2,5,8,11-tetradeoxy-t-erythro-p-t-galacto-dodecofuranoside and methyl 3,7:6,10-dianhydro-12-O-[tert-butyl(diphenyl)silyl]-2,5,8,11-tetradeoxy-L-threo-p-L-ga/acto-dodecofuranoside (50a and 50b): To a stirred solution ketone 49 (350 mg, 0.685 mmol, 1.0 equiv) in THF (35 mL) at 0° C. was added K-Selectride (1.0 M in

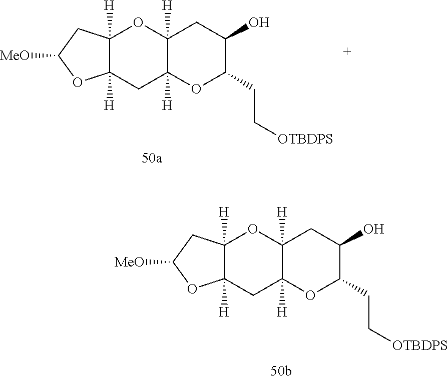

THF, 1.03 mL, 1.03 mmol, 1.5 equiv) dropwise. The resulting mixture was stirred for 20 min before it was quenched by addition 50a 50b of NH₄Cl solution (10 mL, sat. aq.). The layers were separated, and the aqueous layer was extracted with EtOAc (3×20 mL). The combined organic layers were washed with brine (20 mL), dried over Na₂SO₄, and concentrated under reduced pressure. Flash column chromatography (SiO₂, hexanes/EtOAc 3:1, v/v→1:2, v/v) of the residue afforded alcohols 50b (253 mg, 0.493 mmol, 72% yield) and 50a (70.2 mg, 0.137 mmol, 20% yield) as colorless oils, respectively. The configuration of the newly formed chiral center within 50a was established by ¹H nOe spectroscopic studies. 50b (major): R_{f}=0.30 (SiO₂, hexanes/EtOAc 1:1, v/v);

$${[ α ]}_{D}^{23} = + 8.7 ( c = 0.61 , {CHCl}_{3} ) ;$$

FTIR (film): vₘₐₓ 3450, 2930, 2857, 1472, 1428, 1371, 1197, 1170, 1111, 1088, 1060, 1035, 823, 704 cm¹; ¹H NMR (600 MHz, CDCl₃): δ 7.70-7.63 (m, 4H), 7.45-7.34 (m, 6H), 5.17 (dd, J=5.9, 3.2 Hz, 1H), 4.20 (dq, J==10.1, 4.9 Hz, JH), 4.07 (dt, J=8.4, 5.5 Hz, 1H), 3.96 (dd, J==5.6, 2.6 Hz, 1H), 3.84 (dt, J=5.0, 2.4 Hz, 1H), 3.80-3.75 (m, 2H), 3.44 (d, J=1.5 Hz, 1H), 3.35 (s, 3H), 3.34-3.31 (m, 1H), 2.30 (d, J=5.3 Hz, 1H), 2.25 (dd, J=14.5, 5.8 Hz, 1H), 2.08 (dt, J=15.8, 2.8 Hz, 1), 2.06-2.01 (m, 1H), 2.00-1.92 (m, 2H), 1.89-1.81 (m, 2H), 1.75 (ddd, J=13.4, 11.1, 3.7 Hz, 1H), 1.05 (s, 9H)ppm; ¹³C NMR (151 MHz, CDCl₃): δ 135.77, 135.76, 133.6, 133.4, 129.87, 129.85, 127.87, 127.83, 127.8, 104.5, 76.6, 74.2, 71.8, 71.7, 63.8, 62.1, 61.9, 55.7, 41.7, 33.4, 29.4, 27.7, 27.0, 19.2 ppm; HRMS (ESI-TOF) m/z: [M+Na]⁺ Calcd. for C₂₉H₄₀O₆SiNa⁺ 535.2486; Found 535.2481, 50a (minor): R_{f}=0.40 (SiO₂, hexanes/EtOAc 1:1, v/v);

$${[ α ]}_{D}^{23} = + 25.3 ( c = 0.68 , {CHCl}_{3} ) ;$$

FTIR (film): vₘₐₓ 3518, 2951, 2930, 2888, 2858, 1472, 1428, 1389, 1195, 1112, 1096, 1029, 824, 740, 704 cm⁻¹; ¹H NMR (600 MHz, CDCl₃): δ 7.78-7.58 (m, 4H), 7.47-7.32 (m, 6H), 5.21 (dd, J=5.9, 3.4 Hz, 1H), 4.04 (dd, J=9.7, 5.1 Hz, 1H), 3.94 (dd, J=5.5, 2.2 Hz, 1H), 3.85 (dt, J=5.0, 1.8 Hz, 1H), 3.83-3.72 (m, 3H), 3.45 (d, J=10.7 Hz, 1H), 3.40-3.35 (m, 1H), 3.36 (s, 3H), 3.31 (dt, J=4.9, 1.5 Hz, 1H), 2.29 (dd, J=14.9, 6.0 Hz, 1H), 2.18-2.12 (m, 1H), 2.08-2.03 (m, 1H), 2.03-1.98 (m, 1H), 1.94-1.88 (m, 1H), 1.88-1.78 (m, 2H), 1.62 (dddd, J=13.8, 8.6, 6.8, 5.3 Hz, 1H), 1.04 (s, 9H)ppm; ¹³C NMR (151 MHz, CDCl₃): δ 135.72, 135.71, 134.0, 133.7, 129.8, 129.7, 127.79, 127.75, 104.3, 77.4, 71.3, 71.0, 67.0, 61.6, 61.5, 55.8, 42.0, 31.6, 30.6, 29.9, 27.0, 19.3 ppm; HRMS (ESI-TOF) m/z: [M+Na]⁺ Calcd. for C₂₉H₄₀O₆SiNa⁺ 535.2486; Found 535.2490.
Methyl 3,7:6,10-dianhydro-9-azido-12-O-[tert-butyl(diphenyl)silyl]-2,5,8,9,11-pentadeoxy-L-erythro-β-L-galacto-dodecofuranoside (51): To a stirred solution of alcohol 50b (196 mg, 0.382 mmol, 1.0 equiv) in pyridine (3 mL) at 0° C. was added methanesulfonyl chloride (44.3 μL, 0.573 mmol,

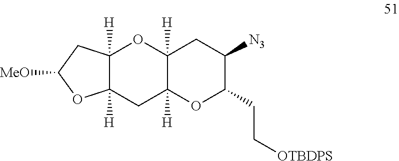

1.5 equiv). The resulting mixture was allowed to warm to 23° C. and stirred for 2 h before it was concentrated under reduced pressure. Flash column chromatography (SiO₂, hexanes/EtOAc 3:1, v/v→1:2, v/v) of the residue afforded methanesulfonate Ms-50b (203 mg, 0.344 mmol, 90% yield) as a colorless oil, which was used in the next step without further characterization.

To a stirred solution methanesulfonate Ms-50b (170 mg, 0.288 mmol, 1.0 equiv) in DMF (10 mL) at 23° C. was added NaN₃ (150 mg, 2.30 mmol, 8.0 equiv). The resulting mixture was heated to 100° C. and stirred at this temperature for 5 h before it was cooled to 0° C. and then diluted with H₂O (15 mL). The layers were separated, and the aqueous layer was extracted with EtOAc (3×30 mL). The combined organic layers were washed with brine (20 μL), dried over Na₂SO₄, and concentrated under reduced pressure. Flash column chromatography (SiO₂, hexanes/EtOAc 5:1, v/v→2:1, v/v) of the residue afforded azide 51 (104 mg, 0.193 mmol, 67%) as a colorless oil. 51: R_{f}=0.60 (SiO₂, hexanes/EtOAc 2:1, v/v;

$${[ α ]}_{D}^{23} = + 37.3 ( c = 1. , {CHCl}_{3} ) ;$$

FT-IR (film): vₘₐₓ 2931, 2105, 1472, 1428, 1263, 1198, 1097, 1030, 987, 870, 823, 703 cm⁻¹; H NMR (600 MHz, CDCl₃): δ 7.69-7.60 (m, 4H), 7.46-7.35 (m, 61H), 5.20 (dd, J=5.9, 2.8 Hz. 1H), 4.21-4.16 (m, 1H), 4.00 (dd, J=5.8, 2.6 Hz, 1H), 3.85 (dt, J=5.1, 2.4 Hz, 1H), 3.79 (dt, J=10.6, 7.21 Hz, 1H), 3.72 (ddd, J=10.6, 7.9, 5.41 Hz, 1H), 3.36-30.30 (m, 5H), 3.05-2.99 (m, 1H), 2.40 (dd, J=14.6, 6.0 Hz, 1H), 2.15 (dt, J=15.7, 2.51 Hz, 1H), 2.08-2.01 (m, 2H), 2.01-1.92 (m, 2H), 1.86 (dt, J=15.7, 5.1 Hz, 1H), 1.72 (dddd, J=13.6, 7.9, 6.8, 5.51 Hz, 1H), 1.05 (s, 9H)ppm; ¹³C NMR (151 MHz, CDCl₃): δ 135.7, 133.9, 133.6, 129.84, 129.82, 127.85, 127.81, 104.7, 76.8, 73.4, 71.4, 68.2, 62.3, 61.2, 55.6, 54.6, 41.7, 33.0, 30.1, 29.6, 27.0, 19.3 ppm; HRMS (ESI-TOF) m/z: [M+Na]⁺ Calcd. for C₂₉H₃₉N₃O₅SiNa⁺ 560.2551; Found 560.2550.
Methyl 3,7:6,10-dianhydro-9-[(tert-butoxycarbonyl)amino]-12-O-[tert-butyl(diphenyl)silyl]-2,5,8,9,11-pentadeoxy-L-ervthro-β-L-galacto-dodecofuranoside (52): A solution of azide 51 (67.1 mg, 0.125 mmol, 1.0 equiv) and Pd/C (30 wt. % Pd, 20 wt. % of 51, 13.4 mg) in MeOH (3 mL) was

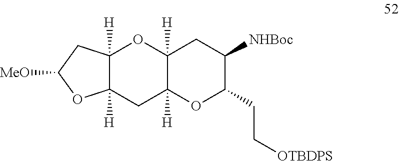

stirred under a hydrogen atmosphere (1 bar) at 23° C. for 2 h. The resulting mixture was then filtered through a pad of Celite® and the filtrate was concentrated under reduced pressure to give the crude corresponding amine, which was used for the next step without further purification.

To a stirred solution of the above obtained amine in THF (3 mL) were added Et₃N (34.8 μL, 0.250 mmol, 2.0 equiv) and Boc₂O (54.6 mg. 0.250 mmol, 2.0 equiv). The resulting mixture was allowed to warm to 23° C. and stirred for 1 h before it was quenched by the addition of NaHCO₃ solution (5 mL, sat. aq.). The layers were separated, and the aqueous layer was extracted with CH₂Cl₂(3×10 mL). The combined organic layers were dried over Na₂SO₄ and concentrated under reduced pressure. Flash column chromatography (SiO₂, hexanes/EtOAc 10:1, v/v→1:1, v/v) of the residue afforded carbamate 52 (60.5 mg, 0.0988 mmol, 79% yield overall) as a colorless oil. 52: R_{f}=0.60 (SiO₂, hexanes/EtOAc 2:1, v/v);

$${[ α ]}_{D}^{23} = + 34. ( c = 0.45 , {CHCl}_{3} ) ;$$

FT-IR (film): vₘₐₓ 3416, 2953, 2930, 2858, 1712, 1500, 1428, 1365, 1257, 1174, 1105, 1093, 1028, 983, 24, 741, 704 cm⁻¹; ^{H} NMR (600 MHz, CDCl₃): δ 7.74-7.62 (m, 41H), 7.46-7.31 (m, 61H), 6.05 (d, J=9.4 Hz, 1), 5.22 (dd, J=5.9, 3.6 Hz, 11H), 3.95-3.89 (m, 2H), 3.85 (dt, J=4.8, 1.9 Hz, 1H), 3.78 (ddd, J=10.5, 9.0, 6.3 Hz, 1H), 3.72 (ddd, J=10.5, 9.4, 5.2 Hz, 1H), 3.44 (d, J=9.5 Hz, 1H), 3.38 (s, 3H), 3.30 (s, 1H), 3.25 (d, J=4.8 Hz, 1H), 2.30 (dd, J=14.7, 6.0 Hz, 1H), 2.10-1.95 (in 3H), 1.89 (dt, J=14.8, 3.9 Hz, 1H), 1.83 (s, 1H), 1.78 (dt, J=16.1, 4.9 Hz, 11H), 1.73-1.64 (m, 11H), 1.41 (s, 91H), 1.04 (s, 9H)ppm; ¹³C NMR (151 MHz, CDCl₃): δ 155.5, 135.8, 135.7, 134.1, 133.8, 129.70, 129.69, 127.8, 127.7, 104.6, 78.7, 76.8, 74.9, 71.5, 70.1, 61.5, 61.1, 56.0, 46.3, 42.1, 31.8, 29.8, 28.6, 28.5, 27.0, 19.3 ppm; HRMS (ESI-TOF) m/z: [M+—Na]⁺ Calcd. for C₃₄H₄₉NO₇SiNa⁺ 634.3171; Found 634.3164.
Methyl 3,7:6,10-dianhydro-9-[(tert-butoxycarbonyl)aminol-2,5,8,9,11-pentadeoxy-L-erythro-β-L-galacto-dodecodialdo-1,4-furanoside (53): To a stirred solution of carbamate 52 (56.3 mg,

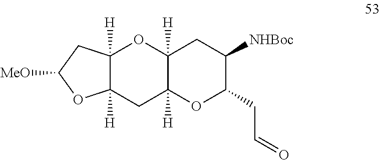 [SMILES: CO[C@@H]1C[C@H]2O[C@H]3C[C@@H](NC(=O)OC(C)(C)C)[C@H](CC=O)O[C@H]3C[C@H]2O1]

0.0920 mmol, 1.0 equiv) in THF (2 mL) at 23° C. was added N,N,N-tributylbutan-1-aninium fluoride (1.0 M in THF; 276 μL, 0.276 mmol 3.0 equiv). The resulting mixture was stirred for 3 h before it was quenched by addition of NH₄Cl solution (5 mL, sat. aq.). The layers were separated, and the aqueous layer was extracted with EtOAc (3×10 mL). The combined organic layers were dried over Na₂SO₄ and concentrated under reduced pressure. Flash column chromatography (SiO₂, hexanes/EtOAc 1:1, v/v→1:5, v/v) of the residue afforded the corresponding alcohol intermediate (34.4 mg, 0.0920 mmol, quantitative yield), which was used in the next step without further characterization.

To a stirred solution of above obtained alcohol (34.4 mg, 0.0920 mmol, 1.0 equiv) in dry CH₂Cl₂ (3 mL) at 23° C. was added Dess-Martin periodinane (97.6 mg, 0.0230 mmol, 2.5 equiv). The resulting mixture was stirred for 1 h before it was diluted with NaHCO₃ (5 mL, sat. aq.) and Na₂S₂O₃ solution (5 mL, sat. aq.). The resulting mixture was vigorously stirred for 2 h. The layers were separated, and the aqueous layer was extracted with CH₂Cl₂ (3×10 mL). The combined organic extracts were dried over Na₂SO₄and concentrated under reduced pressure. Flash column chromatography (SiO₂, hexanes/EtOAc 3:1, v/v→1:1, v/v) of the residue afforded aldehyde 53 (31.4 mg, 0.0846 mmol, 92% yield) as a colorless oil. 53: R_{f}=0.40 (SiO₂, 100% EtOAc);

$${[ α ]}_{D}^{23} = + 124. ( c = 0.5 , {CHCl}_{3} ) ;$$

FT-IR (film): vₘₐₓ=3412, 2972, 2828, 1709, 1504, 1366, 1256, 1236, 1171, 1125, 1103, 1026, 982, 884 cm⁻¹; ¹H NMR (600 MHz, CDCl₃): δ 9.74 (dd, J=5.1, 1.1 Hz, 1H), 6.20 (d, J=9.0 Hz, 1H), 5.25 (dd, J=5.9, 3.5 Hz, 1H), 4.68 (dd, J=11.6, 4.41 Hz, 11H), 3.98 (dd, J=5.3, 2.11 Hz, 1H), 3.92-3.86 (m, 1H), 3.60 (d, J=4.9 Hz, 1H), 3.54-3.49 (m, 1H), 3.44 (s, 1H), 3.40 (s, 3H), 2.83 (ddd, J=15.1, 11.5, 5.1 Hz, 1H), 2.52 (ddd, J=15.1, 4.4, 1.1 Hz, 1H), 2.33 (dd, J=14.7, 5.9 Hz, 1H), 2.25-2.18 (m, 1H), 2.09 (ddd, J=14.8, 5.3, 3.5 Hz, 1H), 2.01-1.92 (m, 3H), 1.43 (s, 9H)ppm; ¹³C NMR (151 MHz, CDCl₃): δ 200.4, 155.5, 104.5, 79.2, 76.9, 72.6, 71.2, 69.6, 61.8, 56.0, 45.8, 43.5, 42.2, 29.6, 28.6, 28.2 ppm; HRMS (ESI-TOF) m/z: [M+Na]⁺ Calcd. for C₁₈H₂₉NO₇Na⁺ 394.1836; Found 394.1832.
Methyl [(1S,2S,4R,7S,9R,10S,12S,14R,16R)-12-{2-[(2S,5S)-5-{2-[(2S,4R,6R)-6-({(2S,3aR,4aS,6R,7S,8aS,9aR)-6-[(tert-butoxycarbonyl)amino]-2-methoxydecahvdrofuro[3,2-b]-pyrano[2,3-e]pyran-7-yl}methyl)-4-methyl-5-methylidenetetrahydro-2H-pyran-2-yl]ethyl}-4-methylidenetetrahydrofura-2-yl]ethyl}-3,8,11,15,17-pentaoxapetacycl[10.4.1.0^{2,7}.0^{9,16}.0^{10,14}-heptadec-4-yl]acetate (55): In a glove box, to a stirred solution of aldehyde 53 (26.3 mg, 0.0708 mmol,

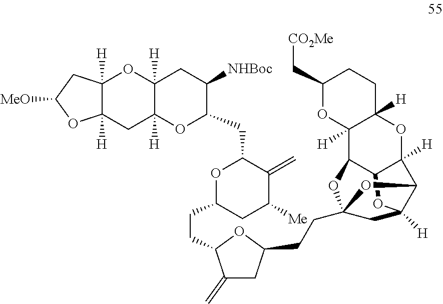 [SMILES: C=C1C[C@H](CC[C@@]23C[C@H]4O[C@H]5[C@@H](O2)[C@H]2O[C@@H](CC(=O)OC)CC[C@@H]2O[C@H]5[C@H]4O3)O[C@H]1CC[C@H]1C[C@@H](C)C(=C)[C@@H](C[C@@H]2O[C@H]3C[C@H]4O[C@H](OC)C[C@H]4O[C@H]3C[C@H]2NC(=O)OC(C)(C)C)O1]

1.5 equiv), fragment IJKLMN (Nicolaou et al., 2021) (8; 35.5 mg, 0.0472 mmol, 1.0 equiv), Et₃N (33.0 μL, 0.236 mmol, 5.0 equiv) and CrCl₂ (17.4 mg, 0.142 mmol, 3.0 equiv) in THF (0.5 mL) at 23° C. were added a premixed solution of ligand (−)-39 (34.8 mg, 0.118 mmol, 2.5 equiv) and NiCl₂ (1.22 mg, 0.00944 mmol, 0.20 equiv) in THF (0.3 mL). The resulting mixture was stirred for 15 h before it was quenched by addition of H₂O (5 mL). The layers were separated, and the aqueous layer was extracted with EtOAc (4×15 mL). The combined organic extracts were washed with brine (10 mL), dried over Na₂SO₄, and concentrated under reduced pressure. Flash column chromatography (SiO₂, hexanes/EtOAc/MeOH 3:1:0, v/v/v→1:10:1, v/v/v) of the residue afforded the corresponding allyl alcohol 54 (33.9 mg, 0.0340 mmol, 72% yield) as a colorless oil.

To a stirred solution of above obtained allyl alcohol 54 (33.9 mg, 0.0340 mmol, 1.0 equiv) in toluene (5 mL) at 23° C. was added DBU (500 μL, 3.35 mmol, 98 equiv, Note: toluene:DBU 10:1, v/v). The resulting mixture was heated to 110° C. and stirred for 1 h before it was allowed to cool to 23° C. and concentrated under reduced pressure. Flash column chromatography (SiO₂, hexanes/EtOAc 3:1, v/v→1:5, v/v) of the residue afforded intermediate 55 (24.5 mg, 0.0272 mmol, 83% yield) as a colorless oil. 55: R_{f}=0.60 (SiO₂, 100% EtOAc);

$${[ α ]}_{D}^{23} = - 11.4 ( c = 0.35 , EtOAc ) ;$$

FT-IR (film): v, 3416, 2928, 2871, 1739, 1710, 1499, 1438, 1365, 1260, 1172, 1129, 1100, 1026, 1079, 901, 883, 755 cm; ¹H NMR (600 MHz, CDCl₃): δ 6.08 (d, J=9.4 Hz, 1H), 5.26 (dd, J=5.9, 3.5 Hz, 1H), 4.96 (d, J=2.2 Hz, 1H), 4.87-4.82 (m, 2H), 4.80 (d, J=1.8 Hz, 114), 4.68 (t, J=4.71 Hz, 1H), 4.60 (t, J=4.5 Hz, 114), 4.42 (dd, J=4.0, 1.9 Hz, 1H), 4.37 (d, J=6.5 Hz, 1H), 4.28 (td, J=10.1, 4.4 Hz, 1H, 4.19 (dd, J=6.6 4.6 Hz, 1H), 4.13-4.08 (m, 1H), 4.06 (dd, J=6.7, 4.2 Hz, 2H), 3.96 (dd, J=5.2, 2.1 Hz, 1H), 3.92-3.89 (in, 1H), 3.81 (dd, J=7.5, 4.5 Hz, 2H), 3.75 (d, J=4.4 Hz, 1H), 3.67 (s, 3H), 3.63-3.56 (m, 1H), 3.56-3.50 (m, 1H), 3.44-3.37 (m, 4H), 2.92 (dd, J=9.6, 1.9 Hz, 1H), 2.64 (dd, J=15.9, 6.8 Hz, 2H), 2.38 (dd, J=15.9, 6.0 Hz, 1H), 2.36-2.18 (m, 4H), 2.15-2.01 (m, 6H), 2.01-1.92 (m, 4H), 1.91-1.87 (m, 1H), 1.85-1.76 (m, 3H), 1.75-1.63 (m, 4H), 1.61-1.51 (m, 2H), 1.51-1.45 (m, 1H), 1.41 (s, 9H), 1.10-1.01 (m, 4H)ppm; ¹³C NMR (151 MHz, CDCl₃): δ 171.7, 155.5, 151.7, 151.1, 110.5, 105.0, 104.6, 104.5, 82.2, 81.2, 79.6, 78.6, 78.2, 77.8, 76.79, 76.75, 76.2, 75.6, 74.6, 74.5, 74.1, 71.6, 70.2, 68.4, 61.4, 56.0, 51.8, 47.1, 46.1, 43.3, 42.2, 40.6, 38.9, 36.2, 35.0, 31.7, 31.4, 30.9, 30.7, 30.1, 30.0, 29.8, 28.6, 283, 18.2 ppm; HRMS (ESI-TOF) m/z: [M+Na]⁺ Calcd. for C₄₈H₇₁NO₁₅Na⁺ 924.4716; Found 924.4702.
Methyl [(1S,2S,4R,7S,9R,10S,12S,14R,16R)-12-{2-[(2S,5S)-5-{2-[(2S,4R,6R)-6-({(2S,3aR,4aS,6R,7S,8aS,9aR)-6-[({[tert-btyl(dimethyl)silyl]oxy}carbonyl)arnino]-2-methoxydecahydrofuro[3,2-b]pyrano[2,3-e]pyran-7-yl}methyl)-4-methyl-5-

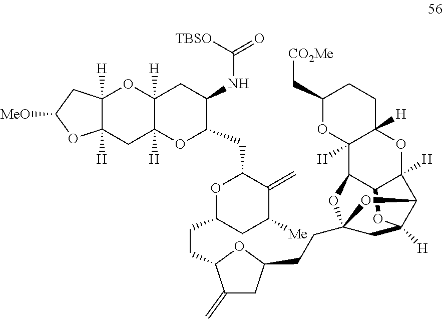 [SMILES: C=C1C[C@H](CC[C@@]23C[C@H]4O[C@H]5[C@@H](O2)[C@H]2O[C@@H](CC(=O)OC)CC[C@@H]2O[C@H]5[C@H]4O3)O[C@H]1CC[C@H]1C[C@@H](C)C(=C)[C@@H](C[C@@H]2O[C@H]3C[C@H]4O[C@H](OC)C[C@H]4O[C@H]3C[C@H]2NC(=O)O[Si](C)(C)C(C)(C)C)O1]

methylidenetetrahydro-2H-pyran-2-yl]ethyl}-4-methylidene-tetralhydrofuran-2-yl]ethyl}-3,8,11,15,17-pentaoXapentacyclo[10.4.1.0^{2,7}.0^{9,16}.0^{10,14}]heptadec-4-yl]-acetate (56): To a stirred solution of intermediate 55 (5.0 mg, 5.5 μmol, 1.0 equiv) in CH₂Cl₂ (3 mL) at 0° C. were added 2,6-lutidine (6.5 μL, 55 μmol, 10 equiv) and TBSOTf (5.1 μL, 28 μmol, 5.0 equiv). The resulting mixture was allowed to warm to 23° C. and stirred for 2 h before it was quenched by the addition of NaHCO₃ solution (3 mL, sat. aq.). The layers were separated, and the aqueous layer was extracted with CH₂Cl₂(3×5 mL). The combined organic layers were dried over Na₂SO₄ and concentrated under reduced pressure. Flash column chromatography (SiO₂, hexanes/EtOAc 10:1, v/v→1:1, v/v) afforded silyl carbarnate 56 (4.7 mg, 4.9 μmol, 89% yield) as a colorless oil. 56: R_{f}=0.80 (SiO₂, 100% EtOAc);

$${[ α ]}_{D}^{23} = - 14.7 ( c = 0.32 , EtOAc ) ;$$

FT-IR (film): vₘₐₓ 3422, 2928, 2858, 1739, 1699, 1505, 1437, 1371, 1260, 1192, 1130, 1079, 1027, 873, 832, 795 cm⁻¹; ¹H NMR (600 MHz, C₆D₆): 66.56 (d, J=9.3 Hz, 1H), 5.44 (dd, J=5.8, 4.2 Hz, 1H), 4.98-4.93 (m, 2H), 4.90 (d, J=2.2 Hz, 1H), 4.83 (d, J=1.9 Hz, 1H), 4.56-4.50 (m, 2H), 4.43 (dd, J=4.0, 1.9 Hz, 1H), 4.36 (t, J=7.0 Hz, 1H), 4.17 (t, J=4.5 Hz, 1H), 4.12 (t, J=4.7 Hz, 1H), 3.98 (dt, J=11.7, 6.3 Hz, 1H), 3.92 (dd, J=6.6, 4.6 Hz, 1H), 3.81 (t, J=6.2 Hz, 1H), 3.79-3.74 (m, 1H), 3.72 (d, J=9.3 Hz, 1H), 3.69 (dd, J=6.6, 4.1 Hz, 2H), 3.53-3.46 (m, 1H), 3.38 (dd, J=4.9, 2.0 Hz, 1H), 3.36 (s, 3H), 3.34 (s, 3H), 3.28 (d, J=4.7 Hz, 1H), 2.71 (brs, 1H), 2.62-2.55 (m, 2H), 2.49-2.43 (m, 1H), 2.35 (dd, J=14.4, 5.9 Hz, 1H), 2.24 (d, J=15.6 Hz, 1H), 2.19-2.11 (m, 3H), 2.09-2.00 (m, 4H), 1.92-1.71 (m, 7H), 1.65-1.57 (m, 1H), 1.51-1.40 (m, 4H), 1.38-1.24 (m, 4H), 1.13-1.04 (m, 2H), 1.00 (s, 9H), 0.97 (d, J=6.5 Hz, 3H), 0.41 (s, 3H), 0.40 (s, 3H)ppm; ¹³C NMR (151 MHz, C₆D₆): δ 170.8, 154.3, 152.4, 151.4, 110.6, 104.7, 104.3, 103.9, 82.0, 80.6, 79.2, 78.3, 77.2, 76.9, 76.7, 76.1, 75.0, 74.6, 74.4, 74.0, 71.7, 69.9, 68.2, 61.3, 55.3, 50.8, 46.9, 46.6, 43.0, 41.9, 40.4, 38.9,35.9,35.6,31.8,31.24, 31.22, 30.5, 30.4, 30.2, 29.7, 27.9, 25.6, 17.8, 17.5, −4.58, −4.62 ppm; HRMS (ESI-TOF) m/z: [M+Na]⁺ Calcd. for C₅₀H₇₇NO₁₅SiNa⁺ 982.4955; Found 982.4939.
(1S,3S,6S,9S,12S,14R,16R,18S,20S,22R,24S,26R,28S,30R,34R,37S,39R,40S,41R,43R,44S)-24-Methoxy-14-methyl-8,15-dimethylidene-2,19,23,27,38,42,45,47,48,49-decaoxa-31-azaundecacyclo[32.9.2.1^{3,40}.1^{3,41}.1^{6,9}.1^{12,16}0.^{18,30}.0^{20,28}0.^{22,26}0.^{37,44}0.^{39,43}]nonatetracntan-32-one (4): To a stirred solution of silyl carbamate 56 (3.8 mg, 4.0 μmol, 1.0 equiv) in THF/H₂O (1.0 mL, 10:1, v/v) at 23° C. was added KF·2H₂O (1.8 mg, 0.020 mmol, 5.0 equiv). The resulting mixture was stirred for

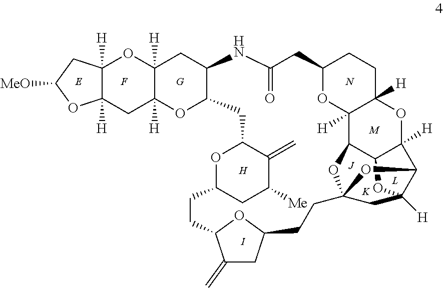

20 min before a LiOH solution (0.2m in H₂O, 0.40 mL) was added. The resulting mixture was stirred for 3 h before it was concentrated under reduced pressure (removal of THF). The residue was diluted with CH₂Cl₂ (5 mL) and aq. HCl solution (0.1 M, 3 mL). The layers were separated, and the aqueous layer was extracted with CH₂Cl₂ (3×5 mL). The combined organic layers were dried over Na₂SO₄, filtered, and the filtrate containing the amino acid intermediate 57 was used for the next step without further purification.

To a stirred solution of the amino acid 57 obtained above in CH₂Cl₂ (~20 mL) at 23° C. were added DMF (4 mL), 3-{[(ethylimino)methylidene]amino}-N,N-dimethylpropan-1-amine (12 mg, 0.080 mmol, 20 equiv) and 3H-[1,2,3]triazolo[4,5-b]pyridin-3-ol (11 mg, 0.080 mmol, 20 equiv). The resulting mixture was stirred for 8 h before it was diluted with NaHCO₃ solution (5 mL, sat. aq.). The layers were separated, and the aqueous layer was extracted with CH₂Cl₂ (3×10 mL). The combined organic layers were dried over Na₂SO₄ and concentrated under reduced pressure. Flash column chromatography (SiO₂, EtOAc/MeOH 50:1, v/v→5:1, v/v) of the residue afforded macrolactam 4 (2.2 mg, 2.9 μmol, 73% yield) as a white foam. 4: R_{f}=0.70 (SiO₂, EtOAc/MeOH 5:1, v/v);

$${[ α ]}_{D}^{23} = - 35.5 ( c = 0.2 , MeOH ) ;$$

FT-IR (film): vₘₐₓ 3424, 2926, 2855, 1667, 1437, 1371, 1263, 1190, 1132, 1101, 1074, 1046, 992, 903, 733 cm⁻¹; ¹H NMR (600 MHz, CD₃OD): 5.15 (dd, J=5.8, 3.4 Hz, 1H), 5.09 (d, J=2.2 Hz, 1H), 5.07 (d, J=2.3 Hz, 1H), 4.91 (s, 1H), 4.84 (d, J=2.0 Hz, 1H), 4.70 (t, J=4.6 Hz, 1H), 4.59 (t, J=4.4 Hz, 1H), 4.41 (d, J=10.91 Hz, 1H), 4.36 (dd, J=4.1, 2.1 Hz, 1H), 4.27 (td, J=10.3, 4.4 Hz, 1H), 4.18 (dd, J=6.5, 4.6 Hz, 1H), 4.13-4.11 (m, 2H), 4.07-4.01 (m, 1H), 4.01 (q, J=3.4 Hz, 1H), 3.90 (dt, J=5.7, 3.01 Hz, 1H), 3.87 (dd, J=6.5, 3.1 Hz, 1H), 3.79 (d, J=9.5 Hz. 2H), 3.74-3.69 (m, 1H), 3.71-3.63 (m, 1H), 3.51-3.46 (m, 1-1), 3.35 (s, 3H), 2.95 (dd, J=9.6, 2.21 Hz, 1H), 2.78-2.71 (m, 1H), 2.42 (dd, J=15.0, 4.0 Hz, 1H), 2.36-2.26 (m, 2H), 2.25-2.15 (m, 3H), 2.13 (dt, J=6.2, 2.8 Hz, 1H), 2.11-2.00 (m, 8H), 1.98 (dd, J=13.2, 5.0 Hz, 1H), 1.94-1.84 (m, 2H), 1.75 (ddd, J=29.0, 12.6, 3.1 Hz, 2H), 1.68-1.55 (m, 3H), 1.55-1.34 (m, 4H), 1.14-1.05 (m, 4H)ppm; ¹³C NMR (151 MHz, CD₃OD): δ 170.8, 154.5, 152.4, 111.1, 105.8, 105.2, 105.1, 83.7, 82.4, 79.2, 78.5, 78.3, 77, 9,77.8,77.0, 76.1, 75.6, 75.1, 75.0, 73.6 (2C), 71.5, 69.7, 64.8, 55.6, 49.6, 47.2, 44.4, 43.3, 42.3, 39.8, 36.8, 36.4, 34.6, 33.0, 32.7, 31.6, 31.4, 30.6, 30.0, 29.0, 18.3 ppm; HRMS (ESI-TOF) m/z: [M+—Na]⁺ Calcd. for C₄₂H₅₉NO₁₂Na⁺ 792.3929; Found 792.3923.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference:
(1) (a) Uemura, D.; Takahashi, K.; Yamamoto, T.; Katayama, C.; Tanaka, J.; Okumura, Y.; Hirata, Y. Norhalichondrin A: An antitumor polyether macrolide from a marine sponge. J. Am. Chem. Soc. 1985, 107, 4796-4798. (b) Hirata, Y.; Uemura, D. Halichondrins—Antitumor polyether macrolides from a marine sponge. Pure Appl. Chem. 1986, 58, 701-710. (c) Litaudon, M.; Hickford, S. J. H.; Lill, R. F.; Lake, R. J.; Blunt, J. W.; Munro, M. H. G. Antitumor polyether macrolides: New and hemisynthetic halichondrins from the New Zealand deep-water sponge Lissodendoryx sp. J Org. Chem. 1997, 62, 1868-1871.
(2) (a) Aicher, T. D.; Buszek, K. R.; Fang, F. G.; Forsyth, C. J.; Jung, S. H.; Kishi, Y.; Matelich, M. C.; Scola, P. M.; Spero, D. M.; Yoon, S. K. Total synthesis of halichondrin B and norhalichondrin B. J. Am. Chem. Soc. 1992, 114, 3162-3164. (b) Jackson, K. L.; Henderson, J. A.; Motoyoshi, H.; Phillips, A. J. A total synthesis of norhalichondrin B. Angew. Chem., Int. Ed. 2009, 48, 2346-2350. (c) Jackson, K. L.; Henderson, J. A.; Phillips, A. J. The halichondrins and E7389. Chem. Rev 2009, 109, 3044-3079. (d) Yamamoto, A.; Ueda, A.; Bremond, P.; Tiseni, P. S.; Kishi, Y. Total synthesis of halichondrin C. J. Am. Chem. Soc. 2012, 134, 893-896. (e) Ueda, A.; Yamamoto, A.; Kato, D.; Kishi, Y. Total synthesis of halichondrin A, the missing member in the halichondrin class of natural products. J. Am. Chem. Soc. 2014, 136, 5171-5176. (f) Yahata, K.; Ye, N.; Ai, Y.; Iso, K.; Kishi, Y. Unified, efficient, and scalable synthesis of halichondrins: Zirconim/nickel-mediated one-pot ketone synthesis as the final coupling reaction. Angew. Chem. Int. Ed. 2017, 56, 10796-10800. (g) Praveen Kumar, V.; Kishi, Y. Total synthesis of halistatins 1 and 2. J. Am. Chem. Soc. 2020, 142, 14743-14749.
(3) (a) Towle, M. J.; Salvato, K. A.; Budrow, J.; Wels, B. F.; Kuznetsov, G.; Aaifs, K. K.; Welsh, S.; Zheng, W.; Seletsky, B. M.; Palme, M. H.; Habgood, G. J.; Singer, L. A.; DiPietro, L. V.; XVang, Y.; Chen, J. J.; Quincy, D. A.; Davis, A.; Yoshimatsu, K.; Kishi, Y.; Yu, M. J.; Littlefield, B. A. In vitro and in vivo anticancer activities of synthetic macrocyclic ketone analogues of halichondrin B. Cancer Res. 2001, 61, 1013-1021. (b) Dabydeen, D. A.; Bumett, J. C.; Bai, R.; Verdier-Pinard, P; Hickford, S. J. H.; Pettit, G. R.; Blunt, J. W.; Munro, M. H. G.; Gussio, R.; Hamel, E. Comparison of the activities of the truncated halichondrin B analog NSC 707389 (E7389) with those of the parent compound and a proposed binding site on tubulin. Mol. Pharmacol. 2006, 70, 1866-1875. (c) Yu, M. J.; Zheng, W.; Seletsky, B. M. From micrograms to grams: Scale-up synthesis of eribulin mesylate. Nat. Prod. Rep. 2013, 30, 1158-1164. (d) Bauer, A. Story of erihulin mesylate: Development of the longest drug synthesis. Top. Heterocycl. Chem. 2016, 44, 209-270. (e) Kawano, S.; Ito, K. Yahata, K.; Kira, K.; Abe, T.; Akagi, T.; Asano, M.; Iso, K.; Sato, Y.; Matsuura, F.; Ohashi, I.; Matsumoto, Y.; Isomura, M.; Sasaki, T.; Fukuyama, T.; Miyashita, Y.; Kaburagi, Y.; Yokoi, A.; Asano, 0.; Owa, T.; Kishi, Y. A landmark in drug discovery based on complex natural product synthesis. Sci. Rep. 2019, 9, 8656.
(4) (a) Wang, Y.; Habgood, G. J.; Christ, W. J.; Kishi, Y; Littlefield, B. A.; Yu, M. J. Structure-activity relationships of halichondrin B analogues: modifications at C.30-C.38. Bioorg. Med. Chem. Lett. 2000, 10, 1029-1032. (b) Seletsky, B. M.; Wang, Y.; Hawkins, L. D.; Palme, M. H., Habgood, G. J.; DiPietro, L. V.; Towle, M. J.; Salvato, K. A.; Wels, B. F.; Aalfs, K. K.; Kishi, Y.; Littlefield, B. A.; Yu, M. J. Structurally simplified macrolactone analogues ofhalichondrin B. Bioorg. Med. Chem. Lett. 2004, 14, 5547-5550. (c) Zheng, W.; Seletsky, B. M.; Palme, M. H.; Lydon, P. J.; Singer, L. A.; Chase, C. E.; Lemelin, C. A.; Shen, Y.; Davis, H.; Tremblay, L.; Towle, M. J.; Salvato, K. A.; Wels, B. F.; Aalfs, K. K.; Kishi, Y.; Littlefield, B. A.; Yu, M. J. Macrocyclic ketone analogues of halichondrin B. Bioorg. Med. Chem. Lett. 2004, 14, 5551-5554. (d) Narayan, S.; Carlson, E. M.; Cheng, H.; Condon, K.; Du, H.; Eckley, S.; Hu, Y.; Jiang, Y.; Kumar, V.; Lewis, B. M.; Saxton, P.; Schuck, E.; Seletsky, B. M.; Tendyke, K.; Zhang, H.; Zheng, W.; Littlefield, B. A.; Towle, M. J.; Yu, M. J. Novel second generation analogs of eribulin. Part II: Orally available and active against resistant tumors in vivo. Bioorg. Med. Chem. Lett. 2011, 21, 1634-1638. (e) Narayan, S.; Carlson, E. M.; Cheng, H.; Du, H.; Hu, Y.; Jiang, Y.; Lewis, B. M.; Seletsky, B. M.; Tendyke, K.; Zhang, H.; Zheng, W.; Littlefield, B. A.; Towle, M. J.; Yu, M. J. Novel second generation analogs of eribulin. Part I: Compounds containing a lipophilic C32 side chain overcome P-glycoprotein susceptibility. Bioorg. Med. Chem. Lett. 2011, 21, 1630-1633.
(5) (a) Cortes, J.; O'Shaughnessy, J.; Loesch, D.; Blim, J. L.; Vahdat, L. T.; Petrakova, K.; Chollet, P.: Manikas, A.: Diéras, V.; Delozier, T.; Vladimirov, V.; Cardoso, F.: Koh. H.; Bougnoux, P.: Dutcus, C. E.; Seegobin, S.; Mir, D.; Meneses, N.; Wanders, J.; Twelves, C. Eribulin monotherapy versus treatment of physician's choice in patients with metastatic breast cancer (EMBRACE): A phase 3 open-label randomised study. Lancet 2011, 377, 914-923. (b) McBride, A.; Butler, S. K. Eribulin mesylate: A novel halichondrin B analogue for the treatment of metastatic breast cancer. Am. J Health, Syst. Pharm. 2012, 69, 745-755. (c) Osgood, C. L.; Chuk, M. K.; Theoret, M. R.; Huang, L.; He. K.; Her, L.; Keegan, P.; Pazdur, R. FDA approval summary: Eribulin for patients with unresectable or metastatic liposarcoma who have received a prior anthracycline-containing regimen. Clin. Cancer Res. 2017, 23, 6384-6389. (d) Chabot, I.; Zhao, Q.; Su, Y. Systematic review of real-world effectiveness of eribulin for locally advanced or metastatic breast cancer. Curr Med. Res. Opin. 2020, 36, 2025-2036.
(6) Nicolaou, K. C.; Pan, S.: Shelke, Y.; Das, D.; Ye, Q.: Lu, Y.; San, S.; Bao, R.; Rigol, S. A reverse approach to the total synthesis of halichondrin B. J Am. Chem. Soc. 2021. 143, 9267-9276.
(7) Nicolaou, K. C.; Pan, S.; Shelke. Y.; Ye, Q.; Das, D.; Rigol, S. A highly convergent total synthesis of norhalichondrin B. J. Am. Chem. Soc. 2021, 143, 20970-20979.
(8) (a) Mahandru, G. M.; Liu, G.; Montgomery, J. Ligand-dependent scope and divergent mechanistic behavior in nickel-catalyzed reductive couplings of aldehydes and alkynes. J. Am. Chem. Soc. 2004, 126, 3698-3699. (b) Baxter. R. D.; Montgomery, J. Mechanistic study of nickel-catalyzed ynal reductive cyclizations through kinetic analysis. J Am. Chem. Soc. 2011, 133, 5728-5731.
(9) Tang, X.-Q.; Montgomery, J. Nickel catalysis in the stereoselective preparation of quinolizidine, pyrrolizidine, and indolizidine alkaloids: Total synthesis of (+)-allopumiliotoxin 267A. J. Am. Chem. Soc. 1999, 121, 6098-6099.
(10) Jana, S.; Roy, S. C. Radical promoted cyclizations of aromatic carbonyl compounds to benzopyrans using titanocene(III) chloride. Tetrahedron Lett. 2006, 47, 5949-5951.
(11) Crowe, W. E.; Rachita, M. J. Titanium-catalyzed reductive cyclization of δ,ϵ-unsaturated ketones and aldehydes. J. Am. Chem. Soc. 1995. 117, 6787-6788.
(12) Shinde, M. P.; Wang, X.; Kang, E. J.; Jang, H.-Y. Platinum-catalyzed hydrogenative cyclization of yne-enones, yne-aldehydes, and yne-dienes. Eur J. Org. Chem. 2009, 6091-6094.
(13) Inokuchi, T.; Kawafuchi, H.; Torii, S. Vanadium(II)-promoted cyclization of 5,6-enals or 5,6-ynals. A stereoselective approach to trans-2-alkyl- or trans-2-alkylidenecyclopentanols. J. Org. Chem. 1991, 56, 4983-4985.
(14) (a) Herlem, D.; Kervagoret, J.; Yu, D.; Khuong-Huu, F.; Kende, A. S. Studies toward the total synthesis of polyoxygenated labdanes: Preliminary approaches. Tetrahedron 1993, 49, 607-618. (b) Herlem, D.; Khuong-Huu, F.; Kende, A. S. Studies toward the total synthesis of diterpenes in the labdane series. III. Synthesis of two epimeric 6,7,8-trihydroxylabdadienes. Tetrahedron 1994, 50, 2055-2070.
(15) (a) Rahim, M. A.; Matsumura, S.; Toshima, K. Deprotection of benzyl ethers using 2,3-dichloro-5,6-dicyano-p-benzoquinone (DDQ) under photoirradiation. Tetrahedron Lett. 2005, 46, 7307-7309. (b) Cavedon, C.; Sletten, E. T.; Madani, A.; Niemeyer, 0.; Seeberger, P. H.; Pieber, B. Visible-light-mediated oxidative debenzylation enables the use of benzyl ethers as temporary protecting groups. Org. Lett. 2021, 23, 514-518.
(16) Mikami, K.: Kasuga, T.; Fujimoto, K.; Nakai, T. Asymmetric [2,3]Wittig rearrangement involving chiral potassium azaenolates. The dramatic influence of the potassium counterion and its complexation with 18-crown-6. Tetrahedron Lett. 1986, 27, 4185-4188.
(17) Yang, Y.-R.; Kim, D.-S.; Kishi, Y. Second generation synthesis of C27-C35 building block of E7389, a synthetic halichondrin analogue. Org. Lett. 2009, 11, 4516-4519.
(18) Ohtsuka, Y.; Oishi, T. An effective synthesis of medium-ring ketones. Tetrahedron Lett. 1979, 50 20, 4487-4490.
(19) Sim, J.; Park, H.; Lim, J.; Yoon, I.; Lim, C.; An, H.; Yun, H.; Choi, H. J.; Suh, Y.-G. Stereoselective synthesis of 1,4,5-tri-cis-guaiane sesquiterpene: First total synthesis of (−)-dendroside C aglycon. Org. Lett. 2018, 20, 586-589.
(20) Koskinen, A. M. P.; Pihko, A. I. Synthesis of DEFG ring system of eneorins. ARKIVOC (Gainesville, FL, U.S.) 2009, 2008, 20-35.
(21) Corbet, M. Applications de la chimie radicalaire des xanthates: Synthése d'alcaloides d'origine marine; Synthése de thiéno[2,3-b]thiopyranones; Synthése de thioéthers aryliques; Approche á la synthése totale du (+)-maritimol, Dissertation, École polytechnique X. Paris, France, October 2009.
(22) (a) Austad, B.; Chase, C. E.; Fang, F. G. Intermediates for the preparation of analogs of halichondrin B. USRE046965, 2004. (b) Zhang, Z. Y.; Huang, J.; Ma, B.; Kishi, Y. Further improvement on sulfonamide-based ligand for catalytic asymmetric 2-haloallylation and allylation. Org. Lett. 2008, 10, 3073-3076.
(23) (a) Marti-Centelles, V; Pandey, M. D.; Burguete, M. I.; Luis, S. V Macrocyclization reactions: The importance of conformational, configurational, and template-induced preorganization. Chem. Rev 2015, 115, 8736-8834. (b) Mortensen, K. T.: Osberger, T. J.; King, T. A.; Sore, H. F.; Spring, D. R. Strategies for the diversity-oriented synthesis of macrocycles. Chem. Rev. 2019, 119, 10288--10317.
(24) Fang, F. G.; Chase, C. E.; Kim, D.-S.; Choi, H.-W. Macrocyclization reactions and intermediates and other fragments useful in the synthesis ofhalichondrin macrolides. U.S. Ser. No. 10/308,661B2, 2019.
(25) (a) Nicolaou, K. C.; Ellery, S. P.: Chen, J. S. Samarium diiodide mediated reactions in total synthesis. Angew. Chem., Int. Ed. 2009, 48, 7140-7165. (b) Yeoman, J. T. S.; Mak, V. W.; Reisman, S. E. A unified strategy to ent-kauranoid natural products: Total syntheses of (−)-trichorabdal A and (−)-longikaurin E. J. Am. Chem. Soc. 2013, 135, 11764--11767.
(26) Takai, K.; Nitta, K.; Fujimura, 0.; Utimoto, K. Preparation of alkylchromium reagents by reduction of alkyl halides with chromium(II) chloride under cobalt catalysis. J. Org. Chem. 1989, 54, 4732-4734.
(27) (a) Furstner, A. Carbon-carbon bond formations involving organochromium(III) reagents. Chem. Rev. 1999, 99, 991-1046. (b) Wessjohann, L. A.; Scheid, G. Rₑcent advances in chromium(II)- and chromium(III)-mediated organic synthesis. Synthesis 1999, 1-36. (c) Takai, K. Addition of organo-chromium reagents to carbonyl compounds. Org. React. 2004, 64, 253-612. (d) Gil, A.; Albericio, F.; Alvarez, M. Role of the Nozaki-I-iyama-Takai-Kishi reaction in the synthesis of natural products. Chem. Rev 2017, 117, 8420-8446.
(28) Austad, B. C.; Calkins, T. L.; Chase, C. E.; Fang, F. G.; Horstmann, T. E.; Hu, Y.; Lewis, B. M.; Niu, X.; Noland, T. A.; Orr, J. D.; Schnaderbeck, M. J.; Zhang, H.; Asakawa, N.; Asai, N.; Chiba, H.; Hasebe, T.; Hoshino, Y.; Ishizuka, H.; Kajima, T.; Kayano, A.; Komatsu, Y.; Kubota, M.; Kuroda, H., Miyazawa, M.; Tagami, K.; Watanabe, T. Commercial manufacture of Halavent: Chemoselective transformations en route to structurally complex macrocyclic ketones. Synlett 2013, 24, 333-337.
(29) Sakaitani, M.; Ohfune, Y. Syntheses and reactions of silyl carbamates. 1. Chemoselective transformation of amino protecting groups via tert-butyldimethylsilyl carbamates. J. Org. Chem. 1990, 55 870-876.
Nicolaou et al., J. Am. Chem. Soc., 143: 9267-9276, 2021.
Mikami et al., Tetrahedron Lett., 27: 4185-4188, 1986
Namba et al., J. Am. Chem. Soc., 126: 7770-7771, 2004.

What is claimed is:

1. A compound of the formula: 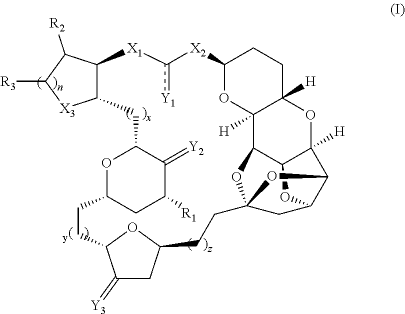
wherein:
X₁, X₂, and X₃ are each independently a covalent bond, alkanediyl_{(C≤8)}, substituted alkanediyl_{(C≤8)}, NRₐ, O, and S; wherein:
Rₐ is hydrogen, alkyl_{(C≤8)}, or substituted alkyl_{(C≤8)};
Y₁, Y₂, and Y₃ are each independently CR_{b}R_{b}′, OR_{b}″, S, or NR_{c}, wherein
R_{b}, R_{b}′, and R_{c} are each independently hydrogen, alkylz_{(C≤8)}, or substituted alkyl_{(C≤8)}; and
R_{b}″ is absent, hydrogen, alkyl_{(C≤8)}, substituted alkyl_{(C≤8)}, acyl_{(C≤8)}, substituted acyl_{(C≤8)},
x, y, and z are each independently 1, 2, or 3;
R₁ is hydrogen, alkyl_{(C≤8)}, or substituted alkyl_{(C≤8)};
R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamino_{(C≤12)}, acyl_{(C≤12)}, acyloxy_{(C≤12)}, amido_{(C≤12)}, or a substituted version thereof; or either two R₃ groups and R₂ and R₃ are taken together to form one or more cycloalkanediyl_{(C≤18)}, arenediyl_{(C≤18)}, heteroarenediyl_{(C≤18)}, heterocycloalkanediyl_{(C≤18)}, or a substituted version thereof; or a group of the formula: —AR₃′, wherein A is alkanediyl_{(C≤8)} or substituted alkanediyl_{(C≤8)}; and R₃′ is NR_{d}C(X₄)NR_{d}′R_{d}″, wherein X₁ is O or NRₑ, wherein Rₑ is hydrogen, alkyl_{(C≤8)}, or substituted alkyl_{(C≤8)}, and R_{d}, R_{d}′, and R_{d}″ are each independently hydrogen, alkyl_{(C≤8)}, or substituted alkyl_{(C≤8)}; and
n is 1 or 2; provided that for each n, then each R₃ is separately selected;
provided that the compound is not eribulin;
or a pharmaceutically acceptable salt thereof.

2. The compound of claim 1 further defined as: 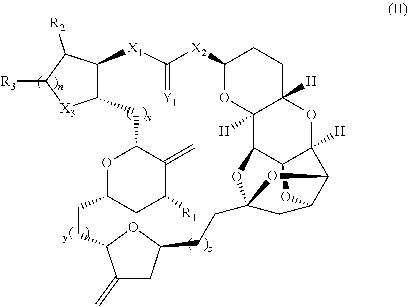
wherein:
X₁, X₂, and X₃ are each independently a covalent bond, alkanediyl_{(C≤8)}) substituted alkanediyl_{(C≤8)}, NRₐ, O, and S; wherein:
Rₐ is hydrogen, alkyl_{(C≤8)}, or substituted alkyl_{(C≤8)};
Y₁ is CR_{b}R_{b}′, O, S, or NR_{c}, wherein
R_{b}, R_{b}′, and_{R} are each independently hydrogen, alkyls), or substituted alkyl_{(C≤8)};
x, y, and z are each independently 1, 2, or 3;
R₁ is hydrogen, alkyl_{(C≤8)}, or substituted alkyl_{(C≤8)};
R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamino_{(C≤12)}, acyl_{(C≤12)}, acyloxy_{(C≤12)}, amido_{(C≤12)}, or a substituted version thereof; or either two R₃ groups and R₂ and R₃ are taken together to form one or more cycloalkanediyl_{(C≤18)}, arenediyl_{(C≤18)}, heteroarenediyl_{(C≤18)}, heterocycloalkanediyl_{(C≤18)}, or a substituted version thereof; or a group of the formula: —AR₃′, wherein A is alkanediyl_{(C≤8)}, or substituted alkanediyl_{(C≤8)}; and R₃′ is NR_{d}C(X₄)NR_{d}′R_{d}″, wherein X₄ is O or NRₑ, wherein Rₑ is hydrogen, alkyl_{(C≤6)}, or substituted alkyl_{(C≤6)}, and R_{d}, R_{d}′, and R_{d}″ are each independently hydrogen, alkyl_{(C≤6)}, or substituted alkyl_{(C≤6)}; and
n is 1 or 2; provided that for each n, then each R₃ is separately selected;
provided that the compound is not eribulin;
or a pharmaceutically acceptable salt thereof.

3. The compound of either claim 1 or claim 2 further defined as: 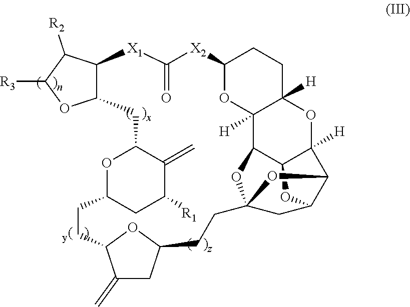
wherein:
X₁ and X₂ is a covalent bond, alkanediyl_{(C≤8)}, substituted alkanediyl_{(C≤8)}, NRₐ, O, and S; wherein:
Rₐ is hydrogen, alkyl_{(C≤8)}, or substituted alkyl_{(C≤8)};
x, y, and z are each independently 1, 2, or 3;
R₁ is hydrogen, alkyl(c s), or substituted alkyl(c s);
R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamino_{(C≤12)}, acyl_{(C≤12)}, acyloxy_{(C≤12)}, amido_{(C≤12)}, or a substituted version thereof; or either two R₃ groups and R₂ and R₃ are taken together to form one or more cycloalkanediyl_{(C≤18)}, arenediyl_{(C≤18)}, heteroarenediyl_{(C≤18)}, heterocycloaikanediyl_{(C≤18)}, or a substituted version thereof; or a group of the formula: —AR₃′, wherein A is alkanediyl_{(C≤8)} or substituted alkanediyl_{(C≤8)}; and R₃′ is NR_{d}C(X₄)NR_{d}′R_{d}″, wherein X₄ is O or NRₑ, wherein Rₑ is hydrogen, alkyl_{(C≤6)}, or substituted alkyl_{(C≤6)}, and R_{d}, R_{d}′ and R_{d}″ are each independently hydrogen, alkyl_{(C≤6)}, or substituted alkyl_{(C≤6)}; and
n is 1 or 2; provided that for each n, then each R₃ is separately selected;
provided that the compound is not eribulin;
or a pharmaceutically acceptable salt thereof.

4. The compound according to any one of claims 1-3 further defined as: 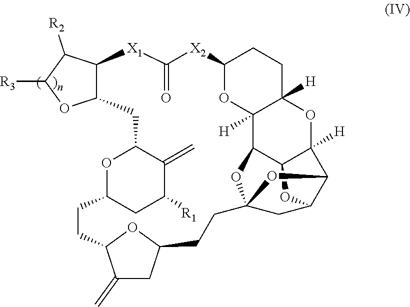
wherein:
X₁ and X₂ is a covalent bond, alkanediyl_{(C≤8)}, substituted alkanediyl_{(C≤8)}, NRₐ, O, and S; wherein:
Rₐ is hydrogen, alkyl_{(C≤8)}, or substituted alkyl_{(C≤8)};
R₁ is hydrogen, alkyl_{(C≤8)}, or substituted alkyl_{(C≤8)};
R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamino_{(C≤12)}, acyl_{(C≤12)}, acyloxy_{(C≤12)}, amido_{(C≤12)}, or a substituted version thereof; or either two R₃ groups and R₂ and R₃ are taken together to form one or more cycloalkanediyl_{(C≤18)}, arenediyl_{(C≤18)}, heteroarenediyl_{(C≤18)}, heterocycloalkanediyl_{(C≤18)}, or a substituted version thereof; or a group of the formula: —AR₃′, wherein A is alkanediyl_{(C≤8)}, or substituted alkanediyl_{(C≤8)}; and R₃′ is NR_{d}C(X₄)NR_{d}′R_{d}″, wherein X₄ is O or NRₑ, wherein Rₑ is hydrogen, alkyl_{(C≤6)}, or substituted alkyl_{(C≤6)}, and R_{d}, R_{d}′, and R_{d}″ are each independently hydrogen, alkyl_{(C≤6)}, or substituted alkyl_{(C≤6)}; and
n is 1 or 2; provided that for each n, then each R₃ is separately selected;
provided that the compound is not eribulin;
or a pharmaceutically acceptable salt thereof.

5. The compound according to any one of claims 1-4 further defined as: 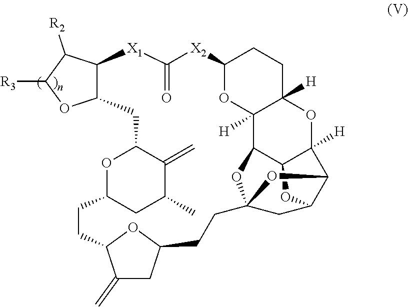
wherein:
X₁ and X₂ is a covalent bond, alkanediyl_{(C≤8)}, substituted alkanediylcs), NRₐ, O, and S; wherein:
Rₐ is hydrogen, alkyl_{(C≤8)}, or substituted alkyl_{(C≤8)};
R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamino_{(C≤12)}, acyl_{(C≤12)}, acyloxy_{(C≤12)}, amido_{(C≤12)}, or a substituted version thereof; or either two R₃ groups and R₂ and R₃ are taken together to form one or more cycloalkanediyl_{(C≤18)}, arenediyl_{(C≤18)}, heteroarenediyl_{(C≤18)}, heterocycloalkanediyl_{(C≤18)}, or a substituted version thereof; or a group of the formula: —AR₃′, wherein A is alkanediyl_{(C≤8)} or substituted alkanediyl_{(C≤8)}; and R₃′ is NR_{d}C(X₄)NR_{d}′R_{d}″, wherein X₄ is O or NRₑ, wherein Rₑ is hydrogen, alkyl_{(C≤6)}, or substituted alkyl_{(C≤6)}, and R_{d}, R_{d}′, and R_{d}″ are each independently hydrogen, alkyl_{(C≤6)}, or substituted alkyl_{(C≤6)}; and
n is 1 or 2; provided that for each n, then each R₃ is separately selected;
provided that the compound is not eribulin;
or a pharmaceutically acceptable salt thereof.

6. The compound of claim 1, wherein Y₂ is CR_{b}R_{b}′.

7. The compound of claim 6, wherein R_{b} is hydrogen.

8. The compound of either claim 6 or claim 7, wherein R_{b}′ is hydrogen.

9. The compound according to any one of claims 1 and 6-8, wherein Y₃ is CR_{b}R_{b}′.

10. The compound of claim 9, wherein R_{b} is hydrogen.

11. The compound of either claim 9 or claim 10, wherein R_{b}′ is hydrogen.

12. The compound according to any one of claims 1, 2, and 6-11, wherein Y₁ is O.

13. The compound according to any one of claims 1, 2, and 6-12, wherein X₃ is 0.

14. The compound according to any one of claims 1-3 and 6-13, wherein x is 1.

15. The compound according to any one of claims 1-3 and 6-14, wherein y is 1.

16. The compound according to any one of claims 1-3 and 6-15, wherein z is 1.

17. The compound according to any one of claims 1-4 and 6-16, wherein R₁ is alkyl_{(C≤8)} or substituted alkyl_{(C≤8)}.

18. The compound of claim 17, wherein R₁ is alkyl_{(C≤8)}.

19. The compound of either claim 17 or claim 18, wherein R₁ is methyl.

20. The compound according to any one of claims 1-19, wherein R₂ is hydroxy.

21. The compound according to any one of claims 1-19, wherein R₂ is alkyl_{(C≤8)} or substituted alkyl_{(C≤8)}.

22. The compound of claim 21, wherein R₂ is alkyl_{(C≤8)}.

23. The compound of either claim 21 or claim 22, wherein R₂ is methyl.

24. The compound according to any one of claims 1-19, wherein R₂ is alkoxy_{(C≤8)} or substituted alkoxy_{(C≤8)}.

25. The compound of claim 24, wherein R₂ is alkoxy_{(C≤8)}.

26. The compound of either claim 24 or claim 25, wherein R₂ is methoxy.

27. The compound according to any one of claims 1-26, wherein R₃ is hydroxy.

28. The compound according to any one of claims 1-26, wherein R₃ is alkyl_{(C≤8)} or substituted alkyl_{(C≤8)}.

29. The compound of claim 28, wherein R₃ is alkyl_{(C≤8)}.

30. The compound of either claim 28 or claim 29, wherein R₃ is methyl.

31. The compound of claim 28, wherein R₃ is substituted alkyl_{(C≤8)}.

32. The compound of either claim 28 or claim 31, wherein R₃ is hydroxymethyl, hydroxypropyl, or dihydroxypropyl.

33. The compound according to any one of claims 1-26, wherein R₃ is alkoxy_{(C≤8)} or substituted alkoxyr_{(C≤8)}.

34. The compound of claim 33, wherein R₃ is alkoxy_{(C≤8)}.

35. The compound of either claim 33 or claim 34, wherein R₃ is methoxy.

36. The compound according to any one of claims 1-26, wherein R₃ is a group of the formula: —AR₃′, wherein A is alkanediyl_{(C≤8)} or substituted alkanediyl_{(C≤8)}; and R₃′ is NR_{d}C(X₄)NR_{d}′Rₐ″, wherein X₄ is O or NRₑ, wherein Rₑ is hydrogen, alkyl_{(C≤6)}, or substituted alkyl_{(C≤6)}, and R_{d}, R_{d}′, and R_{d}″ are each independently hydrogen, alkyl_{(C≤6)}, or substituted alkyl_{(C≤6)}.

37. The compound of claim 36, wherein A is substituted alkanediyl_{(C≤8)}.

38. The compound of claim 37, wherein A is a hydroxy substituted propanediyl.

39. The compound according to any one of claims 36-38, wherein R₃′ is NR_{d}C(X₄)NR_{d}′Rₐ″.

40. The compound of claim 39, wherein X₄ is 0.

41. The compound of either claim 39 or claim 40, wherein R_{d} is hydrogen.

42. The compound according to any one of claims 39-41, wherein R_{d}′ is hydrogen.

43. The compound according to any one of claims 39-42, wherein R_{d}″ is hydrogen.

44. The compound according to any one of claims 1-26, wherein R₃ and R₂ are taken together and are heterocycloalkanediyl_{(C≤18)}, or substituted heterocycloalkanediyl_{(C≤18)}.

45. The compound according to any one of claims 1-44 further defined as: 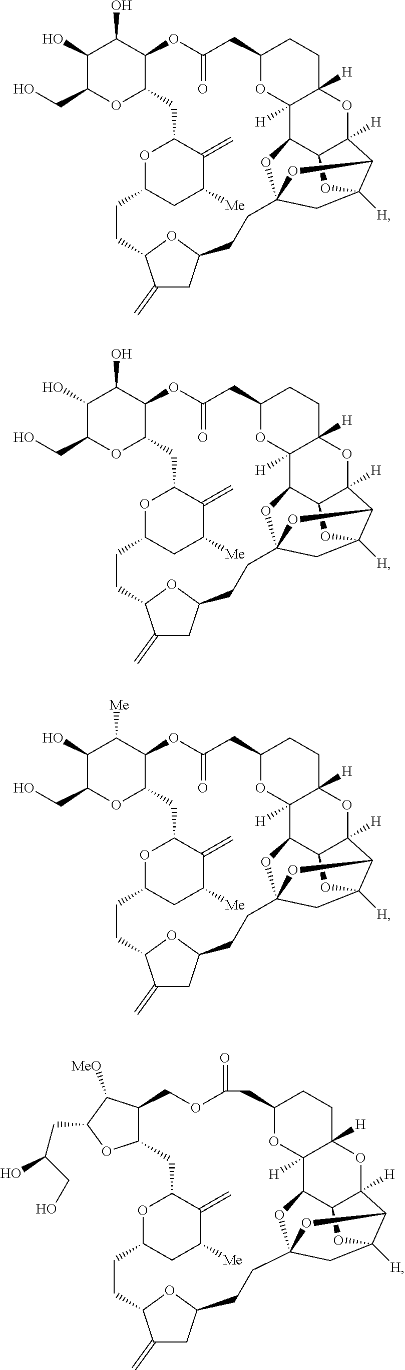 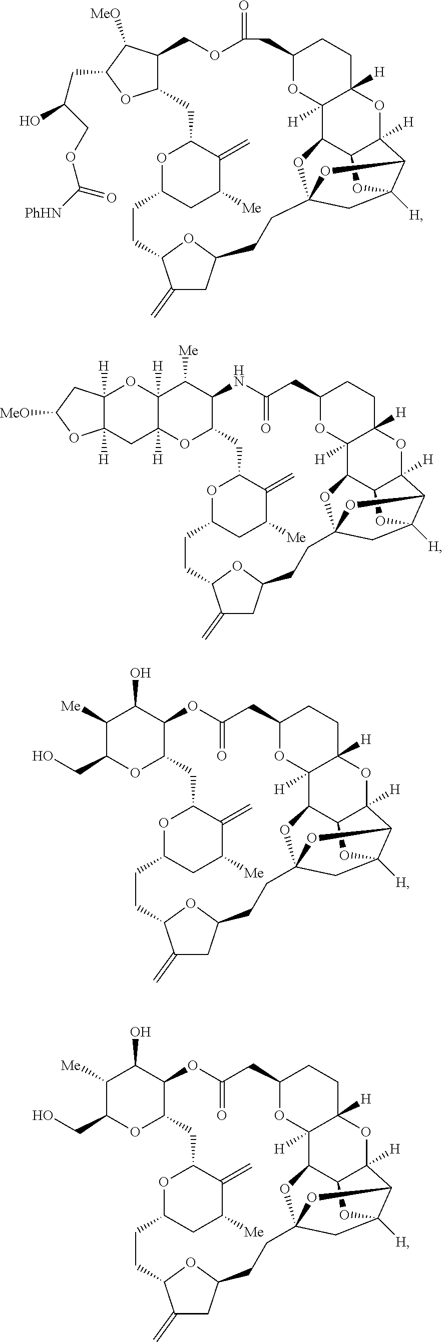 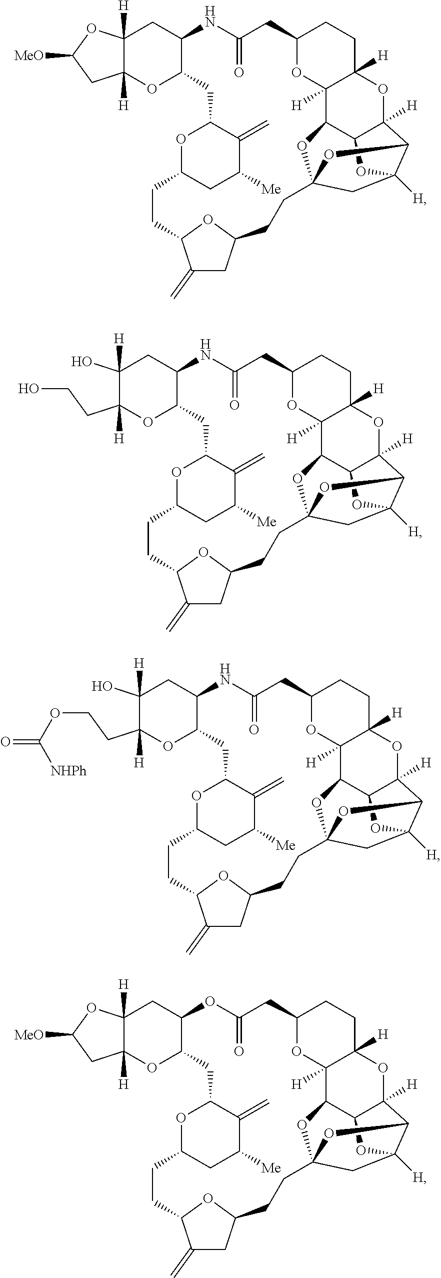 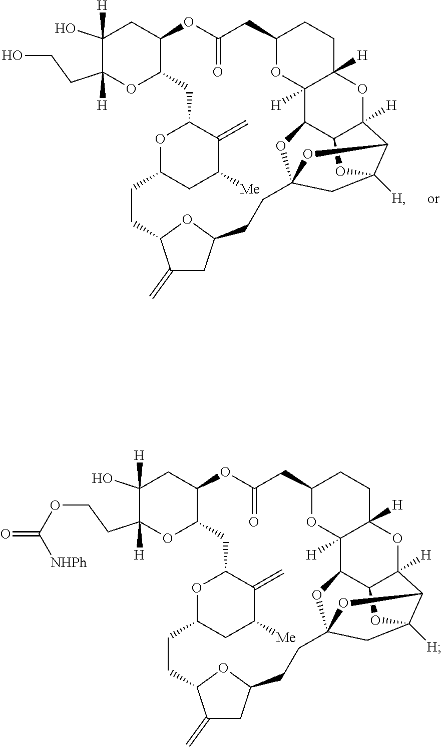
or a pharmaceutically acceptable salt thereof.

46. A pharmaceutical composition comprising: a compound of claim 1 and an excipient.

47. The pharmaceutical composition of claim 46, wherein the composition is formulated for oral administration, administration by injection, or topical administration.

48. A method of treating a disease or disorder in a patient in need thereof comprising administering to the patient a therapeutically effective amount of a compound or pharmaceutically composition according to any one of claims 1-47.

49. The method of claim 48, wherein the disease or disorder is cancer.

50. The method of either claim 48 or claim 49, wherein the disease or disorder is a carcinoma, sarcoma, lymphoma, leukemia, melanoma, mesothelioma, multiple myeloma, or seminoma.

51. The method of either claim 48 or claim 49, wherein the disease or disorder is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

52. The method according to any one of claims 48-51, wherein the method further comprises administering a second therapeutic agent.

53. The method of claim 52, wherein the second therapeutic agent is a second chemotherapeutic agent, surgery, photodynamic therapy, sonodynamic therapy, radiotherapy, or immunotherapy.

54. A method of making erbulin or a compound of claim 1 comprising reacting an intermediate of the formula: 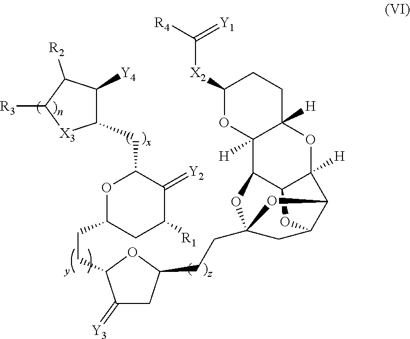
wherein:
Y₁, Y₂, Y₃, X₂, X₃, R₁, R₂, R₃, n, x, y, and z are as defined above;
Y₄ is halo, specifically iodo; and
R₄ is hydrogen, alkoxy_{(C≤12)}, or substituted alkoxy_{(C≤12)};
with a transition metal catalyst to obtain erbulin the compound.

55. An intermediate of the formula: 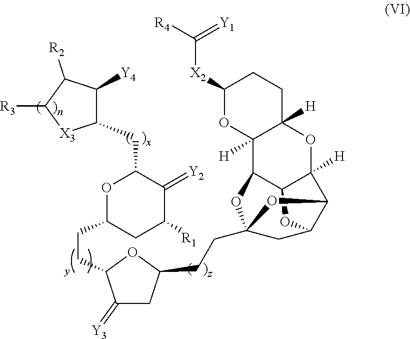
wherein:
X₁, X₂, and X₃ are each independently a covalent bond, alkanediyl_{(C≤8)}, substituted alkanediyl_{(C≤8)}, NRₐ, O, and S; wherein:
Rₐ is hydrogen, alkyl_{(C≤8)}, or substituted alkyl_{(C≤8)};
Y₁, Y₂, and Y₃ are each independently CR_{b}R_{b}′, O, S, or NR_{c}, wherein
R_{b}, R_{b}′, and R_{c} are each independently hydrogen, alkyl_{(C≤8)}), or substituted alkyl_{(C≤8)};
x, y, and z are each independently 1, 2, or 3;
R₁ is hydrogen, alkyl_{(C≤8)}, or substituted alkyl_{(C≤8)};
R₂ and R₃ are each independently alkyl_{(C≤12)}, alkenyl_{(C≤12)}, alkynyl_{(C≤12)}, alkoxy_{(C≤12)}, alkylamino_{(C≤12)}, dialkylamino_{(C≤12)}, acyl_{(C≤12)}, acyloxy_{(C≤12)}, amido_{(C≤12)}, or a substituted version thereof; or either two R₃ groups and R₂ and R₃ are taken together to form one or more cycloalkanediyl_{(C≤18)}, arenediyl_{(C≤18)}, heteroarenediyl_{(C≤18)}, heterocycloalkanediyl_{(C≤18)}, or a substituted version thereof; or a group of the formula: —AR₃′, wherein A is alkanediyl_{(C≤8)} or substituted alkanediyl_{(C≤8)}; and R₃′ is NR_{d}C(X₄)NR_{d}′R_{d}″, wherein X₄ is O or NRₑ, wherein Rₑ is hydrogen, alkyl_{(C≤8)}, or substituted alkyl_{(C≤8)}, and R_{d}, R_{d}′, and R_{d}″ are each independently hydrogen, alkyl_{(C≤8)}, or substituted alkyl_{(C≤8)};
n is 1 or 2; provided that for each n, then each R₃ is separately selected;
Y₄ is halo, specifically iodo; and
R₄ is hydrogen, alkoxy_{(C≤12)}, or substituted alkoxy_{(C≤12)}.
